(19) United States
(12) Patent Application Publication
(10) Pub. No.: US 2024/0238420 A1
(43) Pub. Date: Jul. 18, 2024

(54) COMPOSITIONS AND METHODS FOR TREATING ACUTE MYELOID LEUKEMIA

(71) Applicant: Wuhan University, Wuhan (CN)

(72) Inventors: Ying ZHANG, Wuhan (CN); Guohua CAO, Wuhan (CN); Ruijin JI, Wuhan (CN)

(21) Appl. No.: 18/415,188

(22) Filed: Jan. 17, 2024

(30) Foreign Application Priority Data
Jan. 17, 2023 (WO) PCT/CN2023/072703
Aug. 22, 2023 (CN) 202311057422.8

(51) Int. Cl. A61K 39/00; C07K 14/715; C12N 9/22; C12N 15/11

(52) U.S. Cl. CPC A61K 39/464419; A61K 39/461; A61K 39/4631; A61K 39/4633; C07K 14/7155; C12N 9/22; C12N 15/11; C12N 2310/20

(57) ABSTRACT

The present disclosure describes compositions and methods for treating cancers such as acute myeloid leukemia (AML), in particular relapsed and refractory AML. The method entails administering to the patient an antibody or a chimeric antigen receptor (CAR)-expressing immune cell targeting a molecule such as CD33, CD123, CD117 or CLL-1 following, or concurrently with, transplanting to the patient an engineered stem cell expressing the same molecule but with a mutation disrupting the epitope to the antibody or CAR. Due to the mutation, the engineered stem cell, unlike endogenous hematopoietic cells, is not targeted by the therapy and thus can supply the patient with functional hematopoietic cells and antigens.

### CROSS REFERENCE TO RELATED APPLICATIONS

This application claims the benefit of Chinese application No. PCT/CN2023/072703, filed Jan. 17, 2023, and CN202311057422.8, filed Aug. 22, 2023, the content of each of which is hereby incorporated by reference in its entirety.

### REFERENCE TO AN ELECTRONIC SEQUENCE LISTING

The content of the electronic sequence listing (366244.xml: Size: 797,237 bytes; and Date of Creation: Jan. 16, 2024) is herein incorporated by reference in its entirety.

### BACKGROUND

Acute myeloid leukemia (AML) is a hematological malignancy caused by accumulated mutations in myeloid progenitor cells that cause hyperproliferation and blockage of differentiation, resulting in the accumulation of myeloid blasts in hematopoietic tissues. Despite a high initial response to standard chemotherapy, relapse is common and the prognosis is poor in most AML patients. The conventional chemotherapy drugs cannot fundamentally solve the high occurrences of relapse and drug resistance, and there is a possibility of relapse after hematopoietic stem cell transplantation. Therefore, it is urgent to develop new and improved therapeutic methods.

Chimeric antigen receptor (CAR) T cell therapy uses genetically modified T cells to target and kill cancer cells more specifically and effectively. After T cells are collected from the blood, the cells are engineered to express CARs on their surfaces. CARs can be introduced into T cells using CRISPR/Cas9 gene editing technology. When these allogeneic CAR T cells are injected into patients, the receptors enable the T cells to kill cancer cells.

CD33 (also known as Siglec3, sialic acid-binding Ig-like lectin 3, gp67, or p67) is a member of the Siglec lectin family whose expression is restricted to normal monocytes, granulocytes, hematopoietic progenitors, and immunophenotype-defined hematopoiesis stem cells in the stem cell population. It is expressed on the AML cells of most acute myeloid leukemia patients at both onset and relapse. It functions by binding sialic acid residues of glycoproteins and glycolipids. Anti-CD33 CAR-T cells represent an effective therapeutic option for CD33-expressing malignancies.

CD123 is the alpha chain of interleukin-3 receptor, and CD123 can specifically recognize and bind interleukin-3 (IL-3). IL-3 is mainly produced by helper T cells activated by antigen stimulation, which can promote cell growth and proliferation. It is related to the occurrence of tumors, allergic inflammation, and autoimmune diseases. CD123 is expressed in AML cells from most patients with acute myeloid leukemia. CD123 is also expressed in normal hematopoietic stem cells, functionally related to the differentiation of hematopoietic stem cells. Anti-CD123 CAR-T cells represent an effective therapeutic option for CD123-expressing malignancies.

CD117, also known as mast/stem cell growth factor receptor (SCFR), proto-oncogene c-Kit, tyrosine protein kinase Kit, is a 145-kd transmembrane glycoprotein. Studies in mice with inactivating mutations of c-kit or its ligand, stem cell factor (SCF), have shown that normal functional activity of c-kit is essential for maintaining normal hematopoiesis, melanogenesis, gametogenesis, and cell growth and differentiation. CD117 is expressed on hematopoietic progenitor cells, mast cells, germ cells, interstitial cells of Cajal (ICC), and also highly expressed in AML cells from most AML patients, and thus anti-CD117 CAR-T cells represent an effective treatment for CD117-expressing malignancies.

C-type lectin-like 1 (CLL-1) is also known as MICL, CLEC12A, CLEC-1, dendritic cell-associated lectin 1, and DCAL-2. CLL-1 is a glycoprotein receptor that is a member of a large family of C-type lectin-like receptors involved in immune regulation. Members of this family have diverse functions such as cell adhesion, intercellular signaling, glycoprotein turnover, and roles in inflammation and immune responses. CLL-1 is expressed on hematopoietic cells and primarily on innate immune cells, including monocytes, DCs, pDCs, and granulocyte and myeloid progenitor cells. CLL-1 is also expressed in cancer cells of most acute myeloid leukemia (AML) and myelodysplastic syndrome (MDS) patients. CLL-1 is a leukemia stem cell (LSC)-related surface antigen. Anti-CLL-1 CAR-T cells represent an effective therapeutic option for CLL-1-expressing malignancies.

### SUMMARY

The present disclosure describes compositions and methods for treating cancers such as acute myeloid leukemia (AML). The method entails administering to the patient an antibody drug conjugates or a chimeric antigen receptor (CAR)-expressing immune cell targeting a molecule such as CD33, CD123, CD117 or CLL-1 following, or concurrently with, transplanting to the patient an engineered stem cell expressing the same molecule but with a mutation disrupting the epitope to the antibody or CAR. Due to the mutation, the engineered stem cell, unlike endogenous hematopoietic cells, is not targeted by the therapy and thus can supply the patient with functional hematopoietic cells and antigens.

One embodiment of the present disclosure provides a method for preparing a cancer patient for a therapy, comprising administering to the patient a stem cell expressing a mutant CD33 protein comprising a mutation in an epitope recognized by an anti-CD33 antibody which has reduced binding to the mutant CD33 protein as compared to the corresponding wild-type CD33 protein, wherein the therapy comprises the antibody, an antigen-binding fragment of the antibody, a chimeric antigen receptor (CAR) comprising the antigen-binding fragment, or an immune cell comprising the CAR.

Another embodiment of the present disclosure provides a method for preparing a cancer patient for a therapy, comprising administering to the patient a stem cell expressing a mutant CD123 protein comprising a mutation in an epitope recognized by an anti-CD123 antibody which has reduced binding to the mutant CD123 protein as compared to the corresponding wild-type CD123 protein, wherein the therapy comprises the antibody, an antigen-binding fragment of the antibody, a chimeric antigen receptor (CAR) comprising the antigen-binding fragment, or an immune cell comprising the CAR.

Another embodiment of the present disclosure provides a method for preparing a cancer patient for a therapy, comprising administering to the patient a stem cell expressing a mutant CD117 protein comprising a mutation in an epitope recognized by an anti-CD117 antibody which has reduced binding to the mutant CD117 protein as compared to the corresponding wild-type CD117 protein, wherein the therapy comprises the antibody, an antigen-binding fragment of the antibody, a chimeric antigen receptor (CAR) comprising the antigen-binding fragment, or an immune cell comprising the CAR.

Another embodiment of the present disclosure provides a method for preparing a cancer patient for a therapy, comprising administering to the patient a stem cell expressing a mutant CLL-1 protein comprising a mutation in an epitope recognized by an anti-CLL-1 antibody which has reduced binding to the mutant CLL-1 protein as compared to the corresponding wild-type CLL-1 protein, wherein the therapy comprises the antibody, an antigen-binding fragment of the antibody, a chimeric antigen receptor (CAR) comprising the antigen-binding fragment, or an immune cell comprising the CAR.

In some embodiments, the cancer is leukemia. In some embodiments, the cancer is acute myeloid leukemia (AML).

In some embodiments, the stem cell is a hematopoietic stem and progenitor cell (HSPC).

In some embodiments, the mutant CD33 protein comprises a mutation at one or more residues selected from the group consisting of C41, W60, I105, D112, Y116, F118, P132, W22, G34, R89, N100, N113, and S131 according to SEQ ID NO:1, wherein the mutation is preferably non-conservative. In some embodiments, the anti-CD33 antibody is my9.6 or an antigen-binding fragment thereof.

In some embodiments, the mutant CD33 protein comprises a mutation at one or more residues selected from the group consisting of C41, W60, I105, Y116, and F118 according to SEQ ID NO:1, wherein the mutation is preferably non-conservative. In some embodiments, the anti-CD33 antibody is HM195 or an antigen-binding fragment thereof.

In some embodiments, mutation is introduced to the stem cell with a base editor comprising a gRNA that comprises a spacer sequence selected from the group consisting of SEQ ID NO: 19-144. In some embodiments, mutation is introduced to the stem cell with a prime editor and a pegRNA that comprises a spacer sequence selected from the group consisting of SEQ ID NO:145-228.

In some embodiments, the mutant CD123 protein comprises a mutation at one or more residues selected from the group consisting of 127, L30, M32, W41, E51, C52, S59, P61, R84, P88, F90, S91, and W93 according to SEQ ID NO:2, wherein the mutation is preferably non-conservative. In some embodiments, the anti-CD123 antibody is CSL362 or 32716, or an antigen-binding fragment thereof.

In some embodiments, mutation is introduced to the stem cell with a base editor comprising a gRNA that comprises a spacer sequence selected from the group consisting of SEQ ID NO:229-516. In some embodiments, mutation is introduced to the stem cell with a prime editor and a pegRNA that comprises a spacer sequence selected from the group consisting of SEQ ID NO:517-541.

In some embodiments, the mutant CD117 protein comprises a mutation at one or more residues selected from the group consisting of T67, K69, T71, S81, Y83, T114, T119, and K129 according to SEQ ID NO:3, wherein the mutation is preferably non-conservative. In some embodiments, the anti-CD117 antibody is Ab85 or an antigen-binding fragment thereof.

In some embodiments, the mutant CD117 protein comprises a mutation at one or more residues selected from the group consisting of S236, H238, Y244, S273, T277, and T279 according to SEQ ID NO:3, wherein the mutation is preferably non-conservative. In some embodiments, the anti-CD117 antibody is Ab67 or an antigen-binding fragment thereof.

In some embodiments, mutation is introduced to the stem cell with a base editor comprising a gRNA that comprises a spacer sequence selected from the group consisting of SEQ ID NO:542-758. In some embodiments, mutation is introduced to the stem cell with a prime editor and a pegRNA that comprises a spacer sequence selected from the group consisting of SEQ ID NO:759-801.

In some embodiments, the mutant CLL-1 protein comprises a mutation at one or more residues selected from amino acid residues 142-158 according to SEQ ID NO:4, wherein the mutation is preferably non-conservative. In some embodiments, the anti-CLL-1 antibody is Hu6E7.N54A, or an antigen-binding fragment thereof.

In some embodiments, is introduced to the stem cell with a base editor comprising a gRNA that comprises a spacer sequence selected from the group consisting of SEQ ID NO:802-879. In some embodiments, mutation is introduced to the stem cell with a prime editor and a pegRNA that comprises a spacer sequence selected from the group consisting of SEQ ID NO:880-893.

In some embodiments, the therapy comprises the immune cell that comprises the CAR. In some embodiments, the immune cell is a T cell, an NK cell, or a macrophage.

In some embodiments, the method further comprises administering the therapy to the patient. In some embodiments, the therapy is administered after the stem cell is administered.

In some embodiments, the stem cell is autologous or allogeneic to the patient.

In some embodiments, the patient expresses CD33, CD123, CD117 or CLL-1 in cancer cells.

Also provided, in one embodiment, is a method for treating acute myeloid leukemia (AML) in a patient in need thereof, comprising: (a) editing the genome of a stem cell to introduce a mutation to an epitope of the CD33 protein recognized by an anti-CD33 antibody which has reduced binding to the mutated CD33 protein as compared to the corresponding wild-type CD33 protein, wherein the mutation is at one or more residues selected from the group consisting of C41, W60, I105, D112, Y116, F118, P132, W22, G34, R89, N100, N113, and S131 according to SEQ ID NO:1, and is preferably a non-conservative mutation, (b) transplanting to the patient the edited stem cell, and (c) administering to the patient the antibody, an antigen-binding fragment of the antibody, an antibody-drug conjugate comprising the antibody, a chimeric antigen receptor (CAR) comprising the antigen-binding fragment, or an immune cell comprising the CAR.

Also provided, in one embodiment, is a method for treating acute myeloid leukemia (AML) in a patient in need thereof, comprising: (a) editing the genome of a stem cell to introduce a mutation to an epitope of the CD123 protein recognized by an anti-CD123 antibody which has reduced binding to the mutated CD123 protein as compared to the corresponding wild-type CD123 protein, wherein the mutation is at one or more residues selected from the group consisting of 127, L30, M32, W41, E51, C52, S59, P61, R84, P88, F90, S91, and W93 according to SEQ ID NO:2, and is preferably a non-conservative mutation, (b) transplanting to the patient the edited stem cell, and (c) administering to the patient the antibody, an antigen-binding fragment of the antibody, an antibody-drug conjugate comprising the antibody, a chimeric antigen receptor (CAR) comprising the antigen-binding fragment, or an immune cell comprising the CAR.

Also provided, in one embodiment, is a method for treating acute myeloid leukemia (AML) in a patient in need thereof, comprising: (a) editing the genome of a stem cell to introduce a mutation to an epitope of the CD117 protein recognized by an anti-CD117 antibody which has reduced binding to the mutated CD117 protein as compared to the corresponding wild-type CD117 protein, wherein the mutation is at one or more residues selected from the group consisting of T67, K69, T71, S81, Y83, T114, T119, K129, S236, H238, Y244, S273, T277, and T279 according to SEQ ID NO:3, and is preferably a non-conservative mutation, (b) transplanting to the patient the edited stem cell, and (c) administering to the patient the antibody, an antigen-binding fragment of the antibody, an antibody-drug conjugate comprising the antibody, a chimeric antigen receptor (CAR) comprising the antigen-binding fragment, or an immune cell comprising the CAR.

Also provided, in one embodiment, is a method for treating acute myeloid leukemia (AML) in a patient in need thereof, comprising: (a) editing the genome of a stem cell to introduce a mutation to an epitope of the CLL-1protein recognized by an anti-CLL-1 antibody which has reduced binding to the mutated CLL-1protein as compared to the corresponding wild-type CLL-1protein, wherein the mutation is at one or more residues selected from amino acid residues 142 to 158 according to SEQ ID NO:4, and is preferably a non-conservative mutation, (b) transplanting to the patient the edited stem cell, and (c) administering to the patient the antibody, an antigen-binding fragment of the antibody, an antibody-drug conjugate comprising the antibody, a chimeric antigen receptor (CAR) comprising the antigen-binding fragment, or an immune cell comprising the CAR.

### BRIEF DESCRIPTION OF THE DRAWINGS

FIG. 1A-D. Determination of epitopes of CD33 that mediate the interaction by anti-CD33 scFv clone my9.6 or HM195. (A) Binding affinity of indicated CD33 mutations with anti-CD33 scFV clone my9.6. HEK 293T cells were transfected with wildtype or indicated CD33 mutant and then incubated with anti-CD33 scFV-luciferase fusion proteins, RLU: relative luminescence units. (B) Western blot of CD33 expression. HEK293T cells were transfected with flag tagged wild-type or indicated CD33 mutants. (C) Binding affinity of additional CD33 mutations. RLU: relative luminescence units (D) Western blot of CD33 expression. HEK293T cells were transfected with flag tagged wild-type or indicated CD33 mutants.

FIG. 2A-G. Mutating endogenous CD33 epitopes in HEK 293T cells and human CD34+ HSPCs by base editors and prime editor. (A) Sanger sequencing of base editors targeting W60 of CD33 in CD34+ HSPCs. Red arrow pointed at desired mutation site. (B) Deep sequencing of editing efficiency and editing products. (C) Illustration of Prime editing design and editing efficiency of in HEK293T cells. PE4 was used to target P132 site of CD33. (D-F) Optimization of PE4 with variable nick site (D), PBS lengths (E), RT template lengths (F). (G) Comparison of PE4, PE4max and PEmax in editing P132 into A132 of CD33. HSPCs: hematopioetic stem and progenitor cells

FIG. 3A-B. Determination of epitopes of CD123 that mediate the recognition by scFv clone 32716 or CSL362. (A) Binding affinity of indicated CD123 mutations with anti-CD123 scFv clone 32716 (left panels) or clone CSL362 (right panels). HEK 293T cells were transfected with wildtype or indicated CD123 mutant and then incubated with anti-CD123 scFv-luciferase fusion proteins. (B) Western blot of CD123 expression. HEK293T cells were transfected with flag tagged wild-type or indicated CD123 mutants.

FIG. 4A-C. CD123 combined mutation R84-V85 can reduce the affinity of the CSL362 antibody without affecting expression and the CD123 downstream signaling pathway. (A) Combined mutations at different sites and their binding affinity with CSL362. (B) Immunoprecipitation to detect the expression levels of different combined mutation variants. (C) Immunoprecipitation to detect CD123 downstream signaling pathway pSTAT5. The results show that combined mutations do not affect the normal function of CD123.

FIG. 5A-H. Mutation of endogenous CD123 sites in HEK 293T cells using base editors and prime editors. (A-B) Editing of CD123-R84 (A) or L30 (B) in HEK 293T cells targeted by BE. Arrows indicate the desired mutation sites. (C-E) Deep sequencing of edited efficiency (C, E) and edited products (D, F) in human hematopoietic stem progenitor cells. (E-F) ABE8.8m-mediated single-base editing products have a purity exceeding 90%, while CBE has more off-target products (C-D). (G) Precise editing of the CD123-R84 site using a prime editor. The schematic shows the design of pegRNA. (H) First-generation sequencing shows that both pegRNAs can effectively edit the R84 site. Arrows point to the editing site.

FIG. 6A-D. Hematopoietic stem cells after precise editing of antigen sites have normal myeloid differentiation and proliferative capacity. (A) Myeloid cell count. Cells with precise editing of CD123 R84Q or L30P antigen sites have better cell viability compared to CD123 knockout cells. (B) Myeloid cell morphology. Cells with precise editing of CD123 R84Q or L30P have consistent morphology with the control, while direct knockout of CD123 affects cell morphological changes. (C-D) Myeloid differentiation ratio. Edited hematopoietic stem progenitor cells were subjected to in vitro myeloid differentiation, and differentiation ability was verified using myeloid surface molecules CD11b (C) and CD14 (D). Precise editing of cells does not affect myeloid differentiation.

FIG. 7A-C. Hematopoietic stem cells after precise editing of antigen sites can differentiate into plasmacytoid dendritic cells. (A) Number of plasmacytoid dendritic cells. Cells with precise editing of CD123 R84Q or L30P antigen sites have more plasmacytoid dendritic cells compared to CD123 knockout cells. (B) Ratio of plasmacytoid dendritic cell differentiation. Edited hematopoietic stem progenitor cells were subjected to in vitro differentiation into plasmacytoid dendritic cells, and differentiation ability was verified using CD303 surface molecules. (C) Morphology of plasmacytoid dendritic cells. pDC: plasmacytoid dendritic cell.

FIG. 8A-B. CD123-CAR-T cell targeting experiment. (A) Preparation of CAR-T cells containing CSL362 monoclonal antibody, and co-culturing CAR-T cells with wild-type AML tumor cells, CD123 knockout AML cells, or cells containing CD123-R84Q mutation. (B) The co-cultivation results of hematopoietic stem cells after precise editing through the guide editor and undergoing myeloid differentiation with CAR-T CD123 in vitro were obtained. Compared to the unedited control group, cells after precise editing showed resistance to CAR-T killing, significantly improving cell survival rates.

### DETAILED DESCRIPTION

### Definitions

The term “allogeneic” refers to any material derived from one individual which is then introduced to another individual of the same species, e.g., allogeneic T cell transplantation.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (Clq) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond: two other disulfide bonds connect the heavy chain hinge regions to one another, so that the dimers are connected to one another and the tetramer is formed. Naturally-produced antibodies are also glycosylated, e.g., on the CH2 domain.

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

The term “extracellular ligand-binding domain,” as used herein, refers to an oligo- or polypeptide that is capable of binding a ligand, e.g., a cell surface molecule. For example, the extracellular ligand-binding domain may be chosen to recognize a ligand that acts as a cell surface marker on target cells associated with a particular disease state (e.g., cancer). Examples of cell surface markers that may act as ligands include those associated with viral, bacterial and parasitic infections, autoimmune disease and cancer cells.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered.

The “transmembrane” region or domain is the portion of the CAR that anchors the extracellular binding portion to the plasma membrane of the immune effector cell, and facilitates binding of the binding domain to the target antigen. The transmembrane domain may be a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In one embodiment, the transmembrane domain is the transmembrane domain of CD137. In certain embodiments, the transmembrane domain is synthetic in which case it would comprise predominantly hydrophobic residues such as leucine and valine.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22, CD79a, CD79b and CD66d.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1 BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD3zeta and 4-1 BB, other costimulatory domains are contemplated for use with the CARs described herein. The inclusion of one or more co stimulatory signaling domains may enhance the efficacy and expansion of T cells expressing CAR receptors. The intracellular signaling and costimulatory signaling domains may be linked in any order in tandem to the carboxyl terminus of the transmembrane domain.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the tables below, where a similarity score of 0 or higher indicates conservative substitution between the two amino acids.

A substitution or mutation that is not considered a conservative amino acid substitution/mutation can be referred to as a non-conservative substitution/mutation.

TABLE A
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

A “patient” includes any human who is afflicted with a cancer (e.g., a leukemia). The terms “subject” and “patient” are used interchangeably herein.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. Thus, each zinc finger of a multi-finger ZFP includes a recognition helix region for binding to DNA within a backbone. The term zinc finger DNA binding protein is often abbreviated as zinc finger protein or ZFP. The term “zinc finger nuclease” includes one ZFN as well as a pair of ZFNs (the members of the pair are referred to as “left and right” or “first and second” or “pair”) that dimerize to cleave the target gene.

A “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205. Zinc finger and TALE DNA-binding domains can be “engineered” to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205: 8,586,526; 6,140,081: 6,453,242; and 6,534,261: see also International Patent Publication Nos. WO 98/53058: WO 98/53059: WO 98/53060: WO 02/016536: and WO 03/016496. The term “TALEN” includes one TALEN as well as a pair of TALENs (the members of the pair are referred to as “left and right” or “first and second” or “pair”) that dimerize to cleave the target gene.

CRISPR/Cas (Clustered regularly interspaced short palindromic repeats/CRISPR-associated protein) system has been the most powerful genomic editing tool since its conception for its unparalleled editing efficiency, convenience and the potential applications in living organism. Directed by guide RNA (gRNA), a Cas nuclease can generate DNA double strand breaks (DSBs) at the targeted genomic sites in various cells (both cell lines and cells from living organisms). These DSBs are then repaired by the endogenous DNA repair system, which could be utilized to perform desired genome editing.

Base editors (BE), which integrate the CRISPR/Cas system with the APOBEC (apolipoprotein B mRNA editing enzyme, catalytic polypeptide-like) cytosine deaminase family, were recently developed that greatly enhanced the efficiency of CRISPR/Cas9-mediated gene correction. Through fusion with Cas9 nickase (nCas9) or catalytically dead Cas9 (dCas9), the cytosine (C) deamination activity of rat APOBEC1 (rA1) can be purposely directed to the target bases in genome and to catalyze C to Thymine (T) substitutions at these bases.

Prime editing (PE) is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

### Transplantation of Engineered Tumor Antigen-Expressing Cells

Tumor-associated antigens are commonly targeted for cancer therapies. Ideally, non-cancer cells do not express these antigens and thus would not be killed by the therapies. However, frequently other tissues can also have expression, albeit lower expression sometimes, of these antigens. Such cells therefore can be targeted by the therapy, causing undesired adverse effects.

Example therapies targeting a tumor associated antigen include antibodies, either directly, or through antibody-dependent cellular cytotoxicity (ADCC) or antibody-dependent cellular phagocytosis (ADCP). Another example is chimeric antigen receptor (CAR) T cell therapy which uses genetically modified T cells to target and kill cancer cells.

The present disclosure provides compositions and methods for treating a cancer while reducing adverse effects associated with non-cancerous cells expressing a tumor associated antigen targeted by a therapy. In an illustrative example, a genome editing tool is used to modify the target epitope in a hematopoietic stem and progenitor cell (HSPC) such that the HSPC cannot be bound by the therapeutic antibody or CAR cell, while retaining the normal biological function. When the engineered HSPC is transplanted to a patient that receives the therapy, even if the patient's own HSPC is targeted by the therapy, the transplanted engineered HSPC can supplement the required activity of the HSPC, reducing or avoid the associated toxicities.

According to one embodiment of the present disclosure, provided is a method for preparing a cancer (e.g., leukemia, in particular AML) patient for a therapy. The therapy is designed to specifically target an antigen expressed by the cancer cells, which may include an antibody, an antigen-binding fragment, a chimeric antigen receptor (CAR), or an immune cells (e.g., T cells, NK cells, macrophages, monocytes), or their respective coding sequences. Example tumor associated antigens are known. In some embodiments, the cancer is leukemia. In some embodiments, the leukemia is AML, in particular relapsed and/or refractory AML. For acute myeloid leukemia (AML), the antigen may be CD33, CD123, CD117 or CLL-1, without limitation. In some embodiments, the cancer patient has cancer cells expressing CD33. In some embodiments, the cancer patient has cancer cells expressing CD123. In some embodiments, the cancer patient has cancer cells expressing CD117. In some embodiments, the cancer patient has cancer cells expressing CLL-1.

In some embodiments, the method entails administering to the patient a stem cell that expresses a mutant form of the antigen. In some embodiments, the mutation is at one or more amino acid residues within the epitope of the antigen targeted by the therapy, or at one or more amino acid residues that impact such binding, such by determining the conformation of the epitope. In some embodiments, the mutation does not affect, or at least does not significantly change, the activities of the antigen.

Amino acid residues that are important for the antibody binding have been identified for each of CD33, CD123, CD117 or CLL-1, for a number of commonly used antibodies.

For instance, for CD33, important residues for the binding by antibody my9.6 include C41, W60, I105, D112, Y116, F118, P132, W22, G34, R89, N100, N113, and S131 (residue positions according to CD33 protein sequence as shown in SEQ ID NO: 1). As publicly known, antibody my9.6 has a VH sequence of SEQ ID NO:5 and a VL sequence of SEQ ID NO:6. It is appreciated that an antigen-binding fragment of my9.6 or a CAR molecule that includes the antigen-binding fragment also has the same binding characteristics.

Also for CD33, important residues for the binding by antibody HM195 include C41, W60, I105, Y116, and F118 (residue positions according to CD33 protein sequence as shown in SEQ ID NO:1). As publicly known, antibody HM195 has a VH sequence of SEQ ID NO:7 and a VL sequence of SEQ ID NO:8. It is appreciated that an antigen-binding fragment of HM195 or a CAR molecule that includes the antigen-binding fragment also has the same binding characteristics.

For CD123, important residues for the binding by antibody CSL362 or 32716 include I27, L30, M32, W41, E51, C52, S59, P61, R84, P88, F90, S91, and W93 (residue positions according to CD123 protein sequence as shown in SEQ ID NO:2). As publicly known, antibody CSL362 has a VH sequence of SEQ ID NO:9 and a VL sequence of SEQ ID NO:10; and antibody 32716 has a VH sequence of SEQ ID NO:11 and a VL sequence of SEQ ID NO:12. It is appreciated that an antigen-binding fragment of CSL362 or 32716 or a CAR molecule that includes the antigen-binding fragment also has the same binding characteristics.

For CD117, important residues for the binding by antibody Ab85 include T67, K69, T71, S81, Y83, T114, T119, and K129 (residue positions according to CD117 protein sequence as shown in SEQ ID NO:3). As publicly known, antibody Ab85 has a VH sequence of SEQ ID NO: 13 and a VL sequence of SEQ ID NO:14. It is appreciated that an antigen-binding fragment of Ab85 or a CAR molecule that includes the antigen-binding fragment also has the same binding characteristics.

Also for CD117, important residues for the binding by antibody Ab67 include S236, H238, Y244, S273, T277, and T279 (residue positions according to CD117 protein sequence as shown in SEQ ID NO:3). As publicly known, antibody Ab67 has a VH sequence of SEQ ID NO:15 and a VL sequence of SEQ ID NO:16. It is appreciated that an antigen-binding fragment of Ab67 or a CAR molecule that includes the antigen-binding fragment also has the same binding characteristics.

For CLL-1, important residues for the binding by antibody Hu6E7.N54A include residues 142 to 158 (DSCYFLSDDVQTWQESK) of the CD117 protein sequence as shown in SEQ ID NO:4). As publicly known, antibody Hu6E7.N54A has a VH sequence of SEQ ID NO: 17 and a VL sequence of SEQ ID NO: 18. It is appreciated that an antigen-binding fragment of Hu6E7.N54A or a CAR molecule that includes the antigen-binding fragment also has the same binding characteristics.

In some embodiments, the mutation eliminates or reduces binding of the antigen by a corresponding antibody, antigen-binding fragment or AR. In some embodiments, the mutation is a non-conservative mutation. Examples of non-conservative mutations are provided in Table A, indicated by a negative (<0) similarity score. In some embodiments, only amino acid residues having a similarity score of <−1 are used. In some embodiments, only amino acid residues having a similarity score of <−2, or <−3, or <−4 are used. In some embodiment, the mutation (for a residue that is not alanine) is to alanine. In some embodiments, the mutation is not to alanine. In some embodiments, the mutation is not to cysteine. Example mutations are provided in Table B.

TABLE B
Example Mutations

| | | | | Even more | Most |
|---|---|---|---|---|---|
| | Example mutations | Preferred | More preferred | preferred | preferred |
| W | C/G/P/S/A/T/D/E/N/Q/H/ | G/P/S/A/T/D/E/N/Q/ | G/P/A/T/D/E/N/ | G/P/A/T/D/E/ | G/P/A/T/D/E/ |
| | K/V/M/I/L | H/K/V/M/I/L/F/Y | Q/H/K/V/M/I | N/Q/V/M/I | Q/V/I |
| Y | G/P/S/A/T/D/E/N/Q/K/R/ | G/P/S/A/T/D/E/N/Q/ | G/P/S/A/T/D/E/ | G/P/D/E/Q/K/ | G/P/R |
| | V/M/I/L | H/K/R/V/M/I/L/W | Q/K/R | R | |
| F | C/G/P/S/A/T/D/E/N/Q/H/ | G/P/S/A/T/D/E/N/Q/ | G/P/S/A/T/D/E/ | G/P/A/D/E/N/ | G/P/D/E/Q/K |
| | K/R/V | H/K/R/V/M/W | N/Q/K/R | Q/K/R | |
| L | C/G/P/S/A/T/D/E/N/Q/H/ | G/P/S/A/T/D/E/N/Q/ | G/P/S/D/E/N/K/ | G/D | |
| | K/R/Y/W | H/K/R/Y/W | R | | |
| I | C/G/P/S/A/D/E/N/Q/H/K/ | G/P/S/A/T/D/E/N/Q/ | G/W | W | W |
| | R/Y/W | H/K/R/Y/W | | | |
| M | C/G/P/S/A/T/D/E/Q/H/Y/ | G/P/S/A/T/D/E/N/Q/ | G/D/W | W | |
| | W | H/K/R/F/Y/W | | | |
| V | C/G/P/S/D/E/N/Q/H/K/R/ | G/P/S/A/T/D/E/N/Q/ | W | W | W |
| | F/Y/W | H/K/R/F/Y/W | | | |
| R | C/G/A/T/D/E/V/I/L/F/Y | G/P/S/A/T/D/E/N/V/ | G/L/F/Y | F/Y | Y |
| | | M/I/L/F/Y | | | |
| K | C/G/P/A/V/I/L/F/Y/W | G/P/S/A/T/D/E/H/V/ | L/F/Y/W | F/Y | F |
| | | M/I/L/F/Y/W | | | |
| H | C/G/S/A/T/V/M/I/L/F/W | G/P/S/A/T/K/V/M/I/L/ | W | | |
| | | F/Y/W | | | |
| Q | C/G/S/T/V/M/I/L/F/Y/W | G/P/S/A/T/V/M/I/L/F/ | F/Y/W | F/Y/W | F/W |
| | | Y/W | | | |
| N | C/P/V/I/L/F/Y/W | G/P/A/T/R/V/M/I/L/F/ | L/F/W | F/W | |
| | | Y/W | | | |
| E | C/P/R/V/M/I/L/F/Y/W | G/P/S/A/T/K/R/V/M/I/ | L/F/Y/W | F/Y/W | F/W |
| | | L/F/Y/W | | | |
| D | C/P/R/V/M/I/L/F/Y/W | P/S/A/T/K/R/V/M/I/L/ | M/L/F/Y/W | L/F/Y/W | F/W |
| | | F/Y/W | | | |
| T | C/Q/H/R/M/L/F/Y/W | G/P/D/E/N/Q/H/K/R/ | F/Y/W | W | W |
| | | V/M/I/L/F/Y/W | | | |
| A | C/H/K/R/M/I/L/F/Y/W | D/E/N/Q/H/K/R/V/M/ | F/Y/W | F/W | W |
| | | I/L/F/Y/W | | | |
| S | Q/H/V/M/I/L/F/Y/W | D/E/Q/H/K/R/V/M/I/L/ | L/F/Y | | |
| | | F/Y/W | | | |
| P | C/G/D/E/N/K/V/M/I/L/F/Y/ | G/T/D/E/N/Q/H/K/R/ | L/F/Y/W | F/Y/W | F/Y/W |
| | W | V/M/I/L/F/Y/W | | | |
| G | C/P/Q/H/K/R/V/M/I/L/F/Y/ | P/T/E/N/Q/H/K/R/V/ | R/M/I/L/F/Y/W | L/F/Y/W | F/Y/W |
| | W | M/I/L/F/Y/W | | | |
| C | G/P/A/T/D/E/N/Q/H/K/R/ | G/P/S/A/T/D/E/N/Q/ | G/P/D/E/N/Q/H/ | D/E/N/Q/K/R/ | D/E/Q/K/M/L/ |
| | V/M/I/L/F/W | H/K/R/V/M/I/L/F/Y/W | K/R/M/L/F/W | M/L/F/W | W |

The stem cell being engineered and transplanted can be any stem cell that is able to replace the endogenous cells that can be targeted by the therapy. For AML, for instance, the stem cell may be a hematopoietic stem and progenitor cell (HSPC) or an induced pluripotent stem cell (iPSC), without limitation. Prior to the transplantation, the stem cell may be cultured and/or differentiated. The stem cell, without limitation, may be obtained or derived from a donor subject, or from the patient.

In some embodiments, the therapy includes an corresponding antibody, an antigen-binding fragment of the antibody, a chimeric antigen receptor (CAR) that includes the antigen-binding fragment, or an immune cell that includes the CAR. Methods of preparing antibodies, fragments and CARs are known in the art, such as DNA synthesis, transduction, and expression.

In some embodiments, the CAR is expressed and enclosed in an immune cell for form a CAR-immune cell. In some embodiments, the immune cell is a T cell, an NK cell, or a macrophage, without limitation.

Administration of the therapy is preferably after the stem cell transplantation. In another embodiment, they can be done concurrently. In some embodiments, administration of the therapy, at least one, two or more of the administrations, take place before the stem cell transplantation.

One of ordinary skill in the art would recognize that multiple administrations of the compositions of the disclosure may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

The methods for administering the cell compositions described herein includes any method which is effective to result in reintroduction of ex vivo genetically modified immune effector cells that either directly express a CAR in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct in accordance with the present disclosure and returning the transduced cells into the subject.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield similar results.

### Gene Editing Methods and Edited Cells

The mutations can be introduced to the stem cell with methods known in the art, such as with a zinc finger DNA binding protein, the TALEN technology, a transposon, a retrotransposon, or a CRISPR-based technology, such as base editors and prime editors.

It is commonly known that based editors and prime editors have target sequence requirements and thus it is challenging to design suitable guide RNA sequences. Through trials and errors, the instant inventors were able to design and confirm a number of guide RNA sequences capable to introducing the desired mutations.

A base editor (BE) integrates the CRISPR/Cas system with the APOBEC (apolipoprotein B mRNA editing enzyme, catalytic polypeptide-like) AID (activation-induced cytidine deaminase) family. Through the fusion with the Cas9 nickase (nCas9) or a catalytically dead Cpf1 (dCpf1 also known as dCas12a), the nucleobase deaminase activity of APOBEC/AID family members can be purposely directed to the target bases in the genome and to catalyze base substitutions.

The term “nucleobase deaminase” as used herein, refers to a group of enzymes that catalyze the hydrolytic deamination of nucleobases such as cytidine, deoxycytidine, adenosine and deoxyadenosine. Non-limiting examples of nucleobase deaminases include cytidine deaminases and adenosine deaminases.

“Cytidine deaminase” refers to enzymes that catalyze the irreversible hydrolytic deamination of cytidine and deoxycytidine to uridine and deoxyuridine, respectively. Cytidine deaminases maintain the cellular pyrimidine pool. A family of cytidine deaminases is APOBEC (“apolipoprotein B mRNA editing enzyme, catalytic polypeptide-like”). Members of this family are C-to-U editing enzymes. Some APOBEC family members have two domains, one domain of APOBEC like proteins is the catalytic domain, while the other domain is a pseudocatalytic domain. More specifically, the catalytic domain is a zinc dependent cytidine deaminase domain and is important for cytidine deamination. RNA editing by APOBEC-1 requires homodimerisation and this complex interacts with RNA binding proteins to form the editosome.

Non-limiting examples of APOBEC proteins include APOBEC1, APOBEC2, APOBEC3A, APOBEC3B, APOBEC3C, APOBEC3D, APOBEC3F, APOBEC3G, APOBEC3H, APOBEC4, and activation-induced (cytidine) deaminase (AID).

“Adenosine deaminase”, also known as adenosine aminohydrolase, or ADA, is an enzyme (EC 3.5.4.4) involved in purine metabolism. It is needed for the breakdown of adenosine from food and for the turnover of nucleic acids in tissues.

Non-limiting examples of adenosine deaminases include tRNA-specific adenosine deaminase (TadA), adenosine deaminase tRNA specific 1 (ADAT1), adenosine deaminase tRNA specific 2 (ADAT2), adenosine deaminase tRNA specific 3 (ADAT3), adenosine deaminase RNA specific B1 (ADARB1), adenosine deaminase RNA specific B2 (ADARB2), adenosine monophosphate deaminase 1 (AMPD1), adenosine monophosphate deaminase 2 (AMPD2), adenosine monophosphate deaminase 3 (AMPD3), adenosine deaminase (ADA), adenosine deaminase 2 (ADA2), adenosine deaminase like (ADAL), adenosine deaminase domain containing 1 (ADAD1), adenosine deaminase domain containing 2 (ADAD2), adenosine deaminase RNA specific (ADAR) and adenosine deaminase RNA specific B1 (ADARB1).

Prime editing is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

The pegRNA is capable of identifying the target nucleotide sequence to be edited, and encodes new genetic information that replaces the targeted sequence. The pegRNA consists of an extended single guide RNA (sgRNA) containing a primer binding site (PBS) and a reverse transcriptase (RT) template sequence. During genome editing, the primer binding site allows the 3′ end of the nicked DNA strand to hybridize to the pegRNA, while the RT template serves as a template for the synthesis of edited genetic information.

The fusion protein, in some embodiments, includes a nickase fused to a reverse transcriptase. An example nickase is Cas9 H840A. The Cas9 enzyme contains two nuclease domains that can cleave DNA sequences, a RuvC domain that cleaves the non-target strand and a HNH domain that cleaves the target strand. The introduction of a H840A substitution in Cas9, through which the histidine residue at 840 is replaced by an alanine, inactivates the HNH domain. With only the RuvC functioning domain, the catalytically impaired Cas9 introduces a single strand nick, hence a nickase.

Non-limiting examples of reverse-transcriptases include human immunodeficiency virus (HIV) reverse-transcriptase, moloney murine leukemia virus (M-MLV) reverse-transcriptase and avian myeloblastosis virus (AMV) reverse-transcriptase.

In some embodiments, the prime editing system further includes a single guide RNA (sgRNA) that directs the Cas9 H840A nickase portion of the fusion protein to nick the non-edited DNA strand.

Example gRNA for base editors and example pegRNA for prime editors are provided in Tables 1-4. For instance, for CD33, a gRNA can include a spacer sequence selected from SEQ ID NO:19-144, and pegRNA can include a spacer sequence selected from SEQ ID NO:145-228. For CD123, a gRNA can include a spacer sequence selected from SEQ ID NO: 229-516, and pegRNA can include a spacer sequence selected from SEQ ID NO: 517-541. For CD117, a gRNA can include a spacer sequence selected from SEQ ID NO: 542-758, and pegRNA can include a spacer sequence selected from SEQ ID NO: 759-801. For CLL-1, a gRNA can include a spacer sequence selected from SEQ ID NO: 802-879, and pegRNA can include a spacer sequence selected from SEQ ID NO: 880-893.

In some embodiments, to introduce a mutation at C41 of CD33, the gRNA sequence can include a spacer of a sequence selected from SEQ ID NO:19-33. In some embodiments, to introduce a mutation at W60 of CD33, the gRNA sequence can include a spacer of a sequence selected from SEQ ID NO:34-46. In some embodiments, to introduce a mutation at 1105 of CD33, the gRNA sequence can include a spacer of a sequence selected from SEQ ID NO:47-58. In some embodiments, to introduce a mutation at D112 of CD33, the gRNA sequence can include a spacer of a sequence selected from SEQ ID NO:59-65. In some embodiments, to introduce a mutation at Y116 of CD33, the gRNA sequence can include a spacer of a sequence selected from SEQ ID NO:66-72.

In some embodiments, to introduce a mutation at F118 of CD33, the gRNA sequence can include a spacer of a sequence selected from SEQ ID NO:73-80. In some embodiments, to introduce a mutation at P132 of CD33, the gRNA sequence can include a spacer of a sequence selected from SEQ ID NO:81-89. In some embodiments, to introduce a mutation at W22 of CD33, the gRNA sequence can include a spacer of a sequence selected from SEQ ID NO:90-99. In some embodiments, to introduce a mutation at G34 of CD33, the gRNA sequence can include a spacer of a sequence selected from SEQ ID NO:101-110. In some embodiments, to introduce a mutation at R89 of CD33, the gRNA sequence can include a spacer of a sequence selected from SEQ ID NO:111-118.

In some embodiments, to introduce a mutation at N100 of CD33, the gRNA sequence can include a spacer of a sequence selected from SEQ ID NO:119-128. In some embodiments, to introduce a mutation at N113 of CD33, the gRNA sequence can include a spacer of a sequence selected from SEQ ID NO:129-133. In some embodiments, to introduce a mutation at S131 of CD33, the gRNA sequence can include a spacer of a sequence selected from SEQ ID NO:134-144.

In some embodiments, to introduce a mutation at W22, G34, or C41 of CD33, the pegRNA sequence can include a spacer of a sequence selected from SEQ ID NO: 145-180. In some embodiments, to introduce a mutation at W60 of CD33, the pegRNA sequence can include a spacer of a sequence selected from SEQ ID NO:181-186. In some embodiments, to introduce a mutation at R89, N100, or 1105 of CD33, the pegRNA sequence can include a spacer of a sequence selected from SEQ ID NO:187-212. In some embodiments, to introduce a mutation at D112, N113, Y116, or F118 of CD33, the pegRNA sequence can include a spacer of a sequence selected from SEQ ID NO:213-216. In some embodiments, to introduce a mutation at S131 or P132 of CD33, the pegRNA sequence can include a spacer of a sequence selected from SEQ ID NO:217-228.

In some embodiments, to introduce a mutation at 127 of CD123, the gRNA sequence can include a spacer of a sequence selected from SEQ ID NO:229-263. In some embodiments, to introduce a mutation at L30 of CD123, the gRNA sequence can include a spacer of a sequence selected from SEQ ID NO:264-298. In some embodiments, to introduce a mutation at M32 of CD123, the gRNA sequence can include a spacer of a sequence selected from SEQ ID NO:299-330. In some embodiments, to introduce a mutation at W41 of CD123, the gRNA sequence can include a spacer of a sequence selected from SEQ ID NO:331-345.

In some embodiments, to introduce a mutation at E51 of CD123, the gRNA sequence can include a spacer of a sequence selected from SEQ ID NO:346-376. In some embodiments, to introduce a mutation at C52 of CD123, the gRNA sequence can include a spacer of a sequence selected from SEQ ID NO:377-391. In some embodiments, to introduce a mutation at S59 of CD123, the gRNA sequence can include a spacer of a sequence selected from SEQ ID NO:392-420. In some embodiments, to introduce a mutation at R84 of CD123, the gRNA sequence can include a spacer of a sequence selected from SEQ ID NO:421-435.

In some embodiments, to introduce a mutation at P88 of CD123, the gRNA sequence can include a spacer of a sequence selected from SEQ ID NO:436-450. In some embodiments, to introduce a mutation at F90 of CD123, the gRNA sequence can include a spacer of a sequence selected from SEQ ID NO:451-466. In some embodiments, to introduce a mutation at S91 of CD123, the gRNA sequence can include a spacer of a sequence selected from SEQ ID NO:467-497. In some embodiments, to introduce a mutation at W93 of CD123, the gRNA sequence can include a spacer of a sequence selected from SEQ ID NO:498-516.

In some embodiments, to introduce a mutation at R84, P88, F90, S91, or W93 of CD123, the pegRNA sequence can include a spacer of a sequence selected from SEQ ID NO:517-533. In some embodiments, to introduce a mutation at E51, C52, S59, or P61 of CD123, the pegRNA sequence can include a spacer of a sequence selected from SEQ ID NO:534-537.

In some embodiments, to introduce a mutation at 127, L30, M32 or W41 of CD123, the pegRNA sequence can include a spacer of a sequence selected from SEQ ID NO:538-539. In some embodiments, to introduce a mutation at 127, L30, or M32 of CD123, the pegRNA sequence can include a spacer of SEQ ID NO:540. In some embodiments, to introduce a mutation at 127 or L30 of CD123, the pegRNA sequence can include a spacer of SEQ ID NO:541.

In some embodiments, to introduce a mutation at T67 of CD117, the gRNA sequence can include a spacer of a sequence selected from SEQ ID NO:542-554. In some embodiments, to introduce a mutation at K69 of CD117, the gRNA sequence can include a spacer of a sequence selected from SEQ ID NO:555-561. In some embodiments, to introduce a mutation at T71 of CD117, the gRNA sequence can include a spacer of a sequence selected from SEQ ID NO:562-566. In some embodiments, to introduce a mutation at S81 of CD117, the gRNA sequence can include a spacer of a sequence selected from SEQ ID NO:567-583.

In some embodiments, to introduce a mutation at Y83 of CD117, the gRNA sequence can include a spacer of a sequence selected from SEQ ID NO:584-595. In some embodiments, to introduce a mutation at T114 of CD117, the gRNA sequence can include a spacer of a sequence selected from SEQ ID NO:596-610. In some embodiments, to introduce a mutation at T119 of CD117, the gRNA sequence can include a spacer of a sequence selected from SEQ ID NO:611-623. In some embodiments, to introduce a mutation at K129 of CD117, the gRNA sequence can include a spacer of a sequence selected from SEQ ID NO:624-641.

In some embodiments, to introduce a mutation at S236 of CD117, the gRNA sequence can include a spacer of a sequence selected from SEQ ID NO:642-661. In some embodiments, to introduce a mutation at H238 of CD117, the gRNA sequence can include a spacer of a sequence selected from SEQ ID NO:662-666. In some embodiments, to introduce a mutation at Y244 of CD117, the gRNA sequence can include a spacer of a sequence selected from SEQ ID NO:667-701. In some embodiments, to introduce a mutation at S273 of CD117, the gRNA sequence can include a spacer of a sequence selected from SEQ ID NO:702-738. In some embodiments, to introduce a mutation at T277 of CD117, the gRNA sequence can include a spacer of a sequence selected from SEQ ID NO:739-751. In some embodiments, to introduce a mutation at T279 of CD117, the gRNA sequence can include a spacer of a sequence selected from SEQ ID NO:752-758.

In some embodiments, to introduce a mutation at T67, K69, T71, S81, or Y83 of CD117, the pegRNA sequence can include a spacer of a sequence selected from SEQ ID NO:759-769. In some embodiments, to introduce a mutation at T114, T119, or K129 of CD117, the pegRNA sequence can include a spacer of a sequence selected from SEQ ID NO:770-791.

In some embodiments, to introduce a mutation at S236, H238, or Y244 of CD117, the pegRNA sequence can include a spacer of a sequence selected from SEQ ID NO:792-796. In some embodiments, to introduce a mutation at S273, T277, or T279 of CD117, the pegRNA sequence can include a spacer of a sequence selected from SEQ ID NO:797-801.

In some embodiments, to introduce a mutation at one of the residue of 142 to 158 (DSCYFLSDDVQTWQESK: of SEQ ID NO:4, such as D142, S143, C144, Y145, F146, L147, S148, D149, D150, V151, Q152, T153, W154, Q155, E156, S157, or K158) of CLL-1, the gRNA sequence can include a spacer of a sequence selected from SEQ ID NO:802-879.

In some embodiments, to introduce a mutation at one of the residue of 142 to 158 (DSCYFLSDDVQTWQESK: of SEQ ID NO:4, such as D142, S143, C144, Y145, F146, L147, S148, D149, D150, V151, Q152, T153, W154, Q155, E156, S157, or K158) of CLL-1, the pegRNA sequence can include a spacer of a sequence selected from SEQ ID NO:880-893.

One embodiment provides a mutant CD123 protein comprising a mutation at residue R84 (position according to SEQ ID NO:2). In some embodiments, the mutation is to an amino acid residue that is not lysine. In some embodiments, the mutation is non-conservative. In some embodiments, the mutation is to glutamine (Q), asparagine (N) or histidine (H). In some embodiments, the mutant CD123 protein has reduced binding to an anti-CD123 antibody as disclosed herein, as compared to the wild-type CD123 protein.

In some embodiments, the mutation is R84Q. In some embodiments, the mutation is R84N. In some embodiments, the mutation is R84H.

In some embodiments, the mutant CD123 protein further comprises a mutation at residue V85 (position according to SEQ ID NO:2). In some embodiments, the mutation at residue V85 is to methionine (M), isoleucine (I), leucine (L), alanine (A), cysteine (C), glycine (G), or threonine (T). In some embodiments, the mutation is V85I. In some embodiments, the mutation is V85M.

In some embodiments, the mutations in CD123 are selected from the group consisting of R84Q and V85I, R84Q and V85M, R84H and V85I, and R84H and V85M. In some embodiments, the mutant CD123 protein comprises an amino acid sequence selected from the group consisting of SEQ ID NO: 894, 895, 896 and 897.

Also provided are polynucleotides encoding the mutant CD123 protein of the present disclosure, and cells comprising the mutant CD123 protein or a polynucleotide encoding the mutant CD123 protein.

### EXAMPLES

### Example 1. Identification and Editing of CD33 Epitope

This example identified the potential epitope of the CD33 protein for antibodies my9.6 or HM195, and designed mutants that abolished the antibody binding.

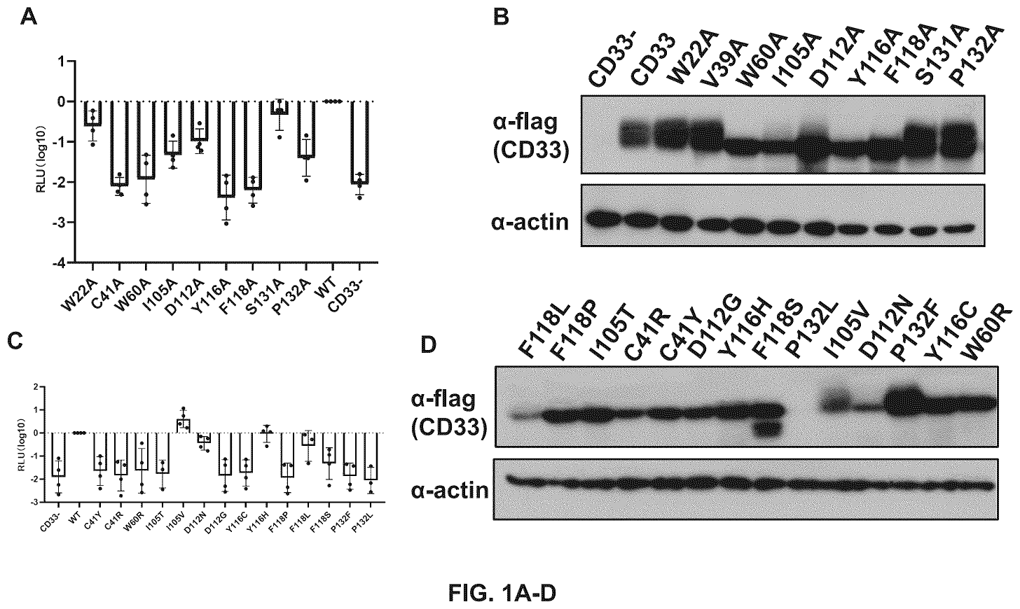

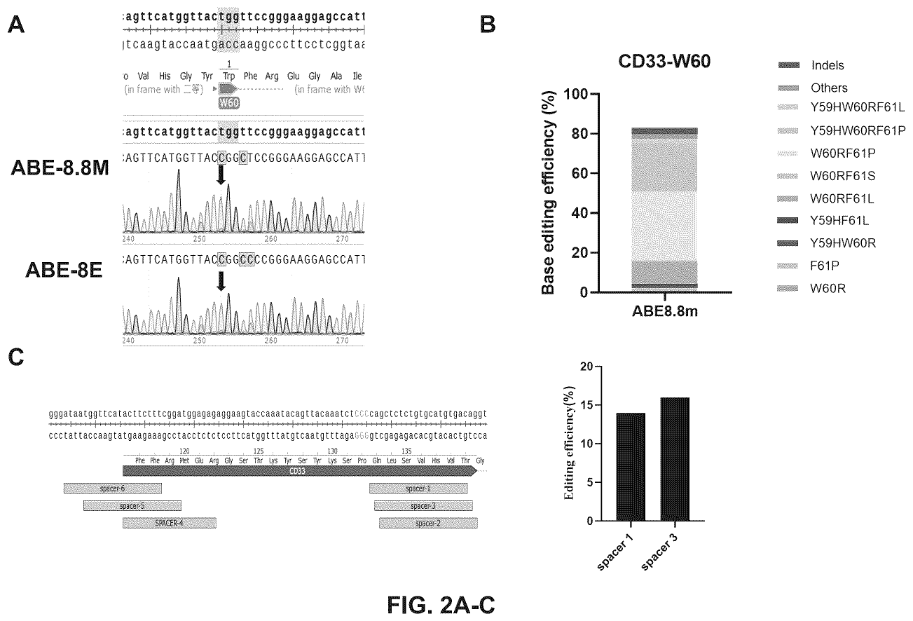

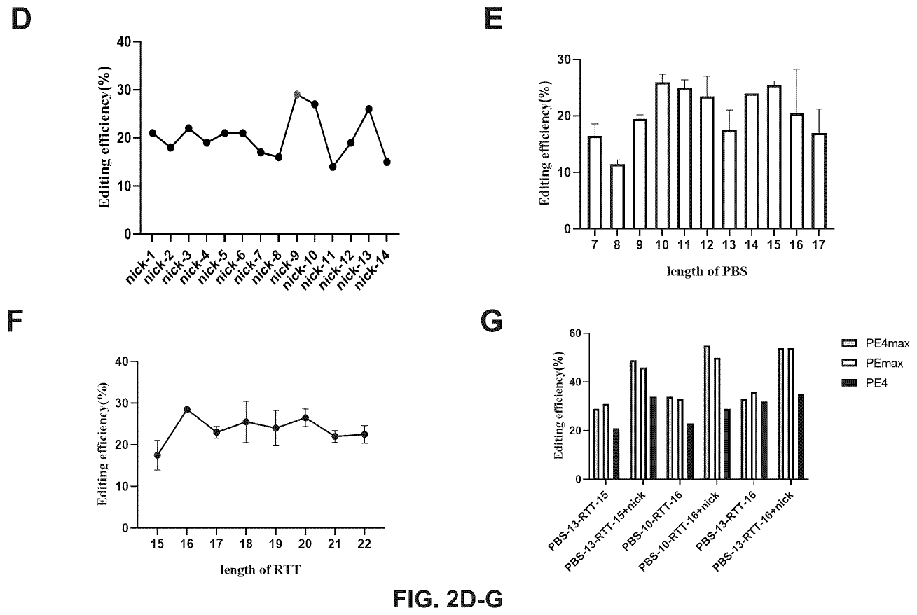

HEK 293T cells were transfected with wildtype or certain CD33 mutants and then incubated with anti-CD33 scFV-luciferase fusion proteins. The mutated residues included W22, C41, W60, I105, D112, Y116, F118, S131, and P132. The tested antibodies included my9.6 and HM195. FIGS. 1A and 1C show the binding affinity of the indicated CD33 mutations with anti-CD33 scFV of antibody my9.6 (RLU: relative luminescence units). FIGS. 1B and 2D present Western blot images of CD33 expression from the transfected cells. These figures clearly show that some of the mutations significantly reduced the binding and thus are within the epitope.

CRISPR-based base editors and prime editors were used to generate mutation at W60 of the CD33 protein. Sanger sequencing confirmed the W60R mutation in the CD34+ HSPCs. In FIG. 2A, red arrow pointed at desired mutation site. The editing efficiency for editing products is shown in FIG. 2B, including Y59h/W60R/F61L, Y59H/W60R/F61P, W60R/F61P, W60R/F61S, W60R/F61L, Y59H/F61L, Y59H/F61L, Y69H/W60R, F61P and W60R. It is clear that the vast majority of the products contained the desired W60R mutation.

FIG. 2C illustrates the prime editing design (locations of spacers for PE4 for mutating P132 to A) and their editing efficiency in HEK293T cells. The editing was further optimized with variable nick sites (FIG. 2D), PBS lengths (FIG. 2E), or RT template lengths (FIG. 2F). In FIG. 2G, the editing efficiencies of PE4, PE4max and PEmax in editing P132 into A of CD33 were compared, all of which were high.

### Example 2. Identification and Editing of CD123 Epitope

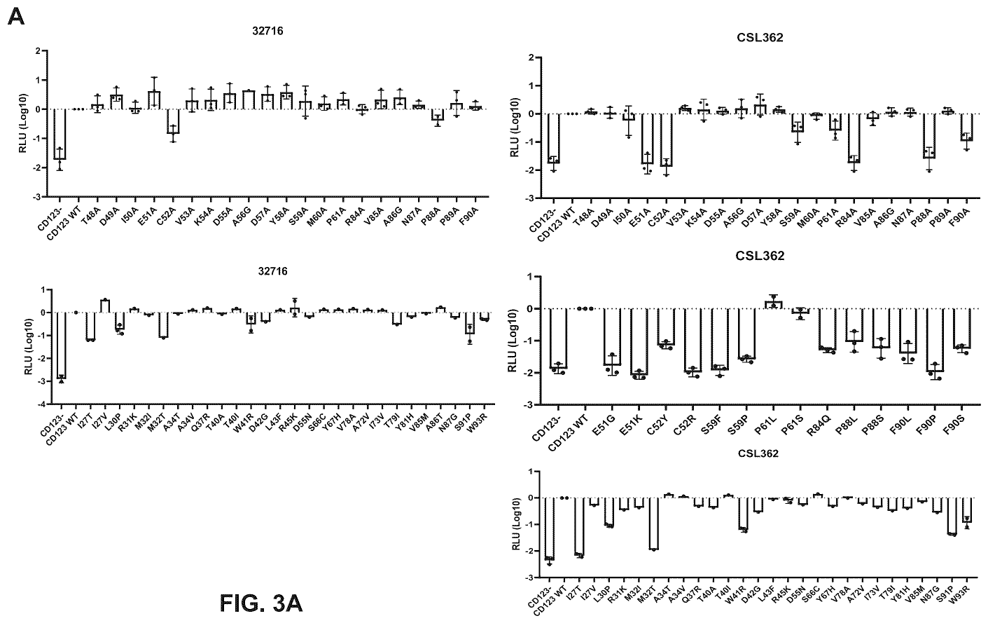

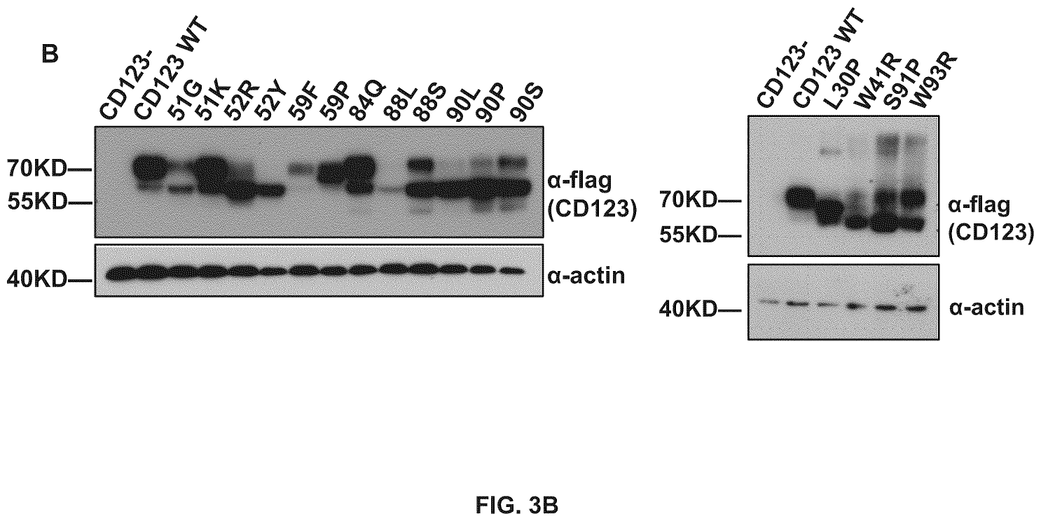

Like in Example 1, the epitopes of CD123 that mediate the recognition by scFv of clone 32716 or CSL362 were determined. HEK 293T cells were transfected with wildtype or indicated CD123 mutant and then incubated with anti-CD123 scFv-luciferase fusion proteins. The binding affinity of indicated CD123 mutations with anti-CD123 scFv clone 32716 (left panels) or clone CSL362 (right panels) is shown in FIG. 3A. The expression of these mutants was confirmed by Western blot (FIG. 3B).

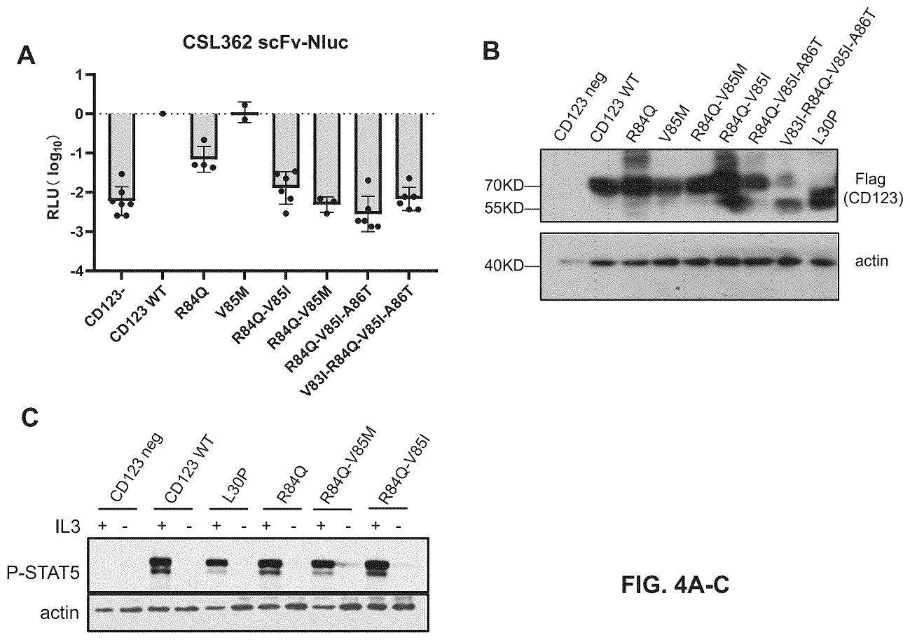

Further reduction of affinity with the scFv was effectively achieved by introducing combined mutations, such as R84Q-V85I or R84Q-V85M, resulting in a 100-fold decrease (FIG. 4A). The introduction of combined mutations did not affect the normal folding and expression of the CD123 protein (FIG. 4B). Validation of downstream functions of the CD123 protein revealed that both combined mutations and single-point mutations did not affect the activation of downstream signaling pathways (FIG. 4C), suggesting that these mutations can specifically reduce interaction with antibodies without affecting the normal protein function.

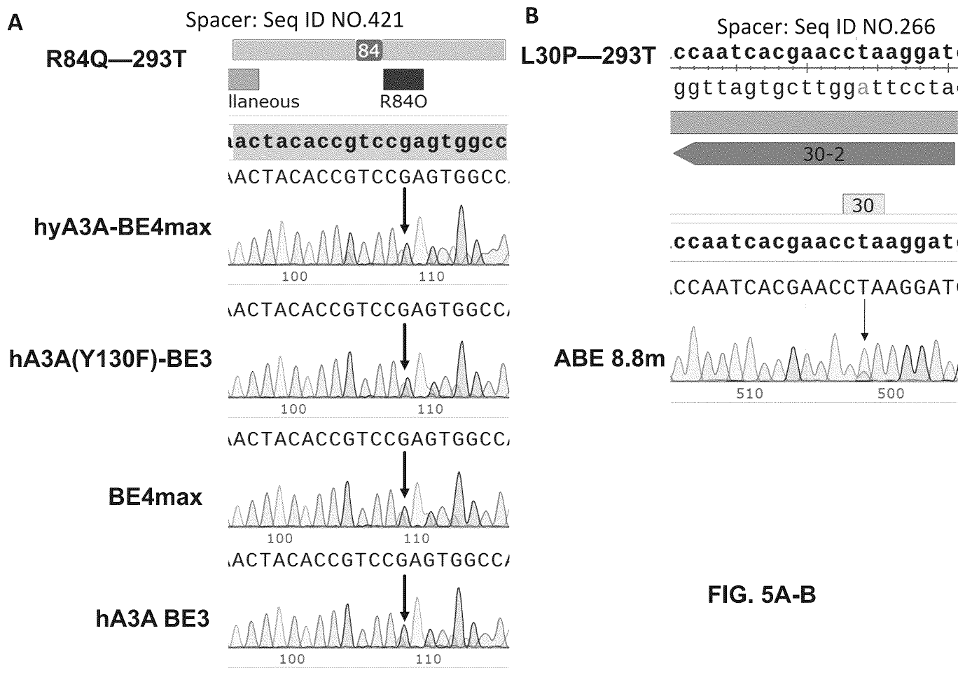

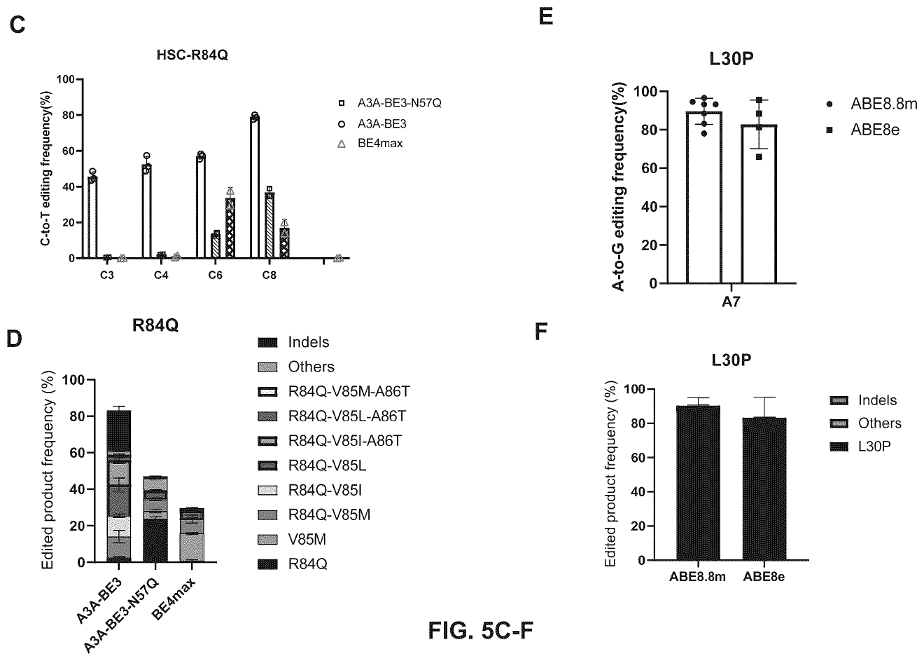

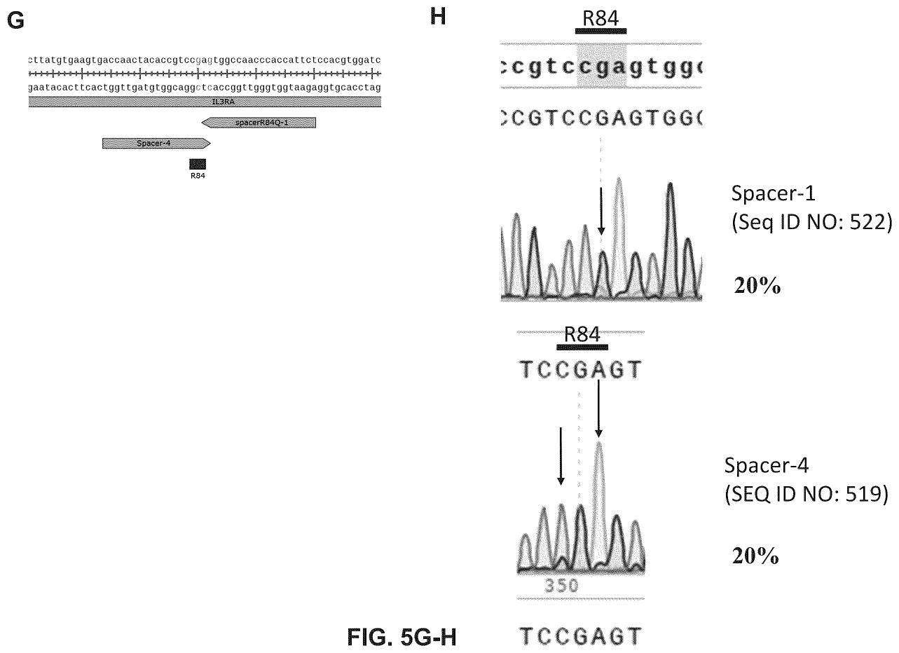

Precise editing of the CD123 antigen site was performed in HEK 293T cells and human hematopoietic stem progenitor cells using base editors and prime editors, respectively. Editing efficiency was confirmed through Sanger sequencing (R84 in FIG. 5A or L30 in FIG. 4B). Deep sequencing results showed the editing efficiency of each editing product (FIG. 5C-F). As shown in FIG. 5G-H, prime editors efficiently and precisely edited the CD123-R84 site. Arrows indicate the editing sites.

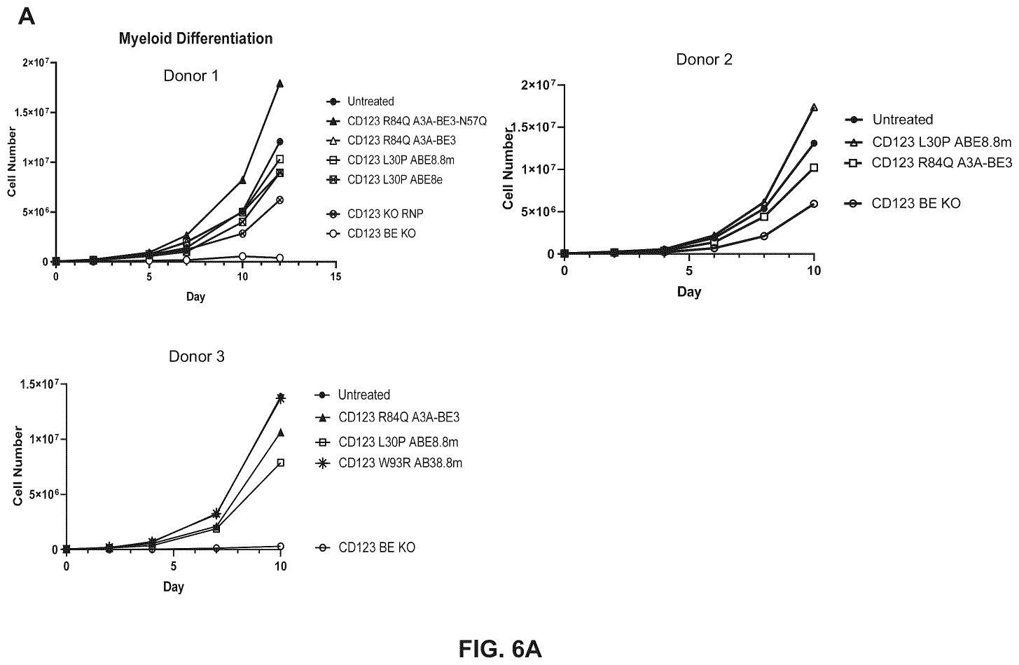

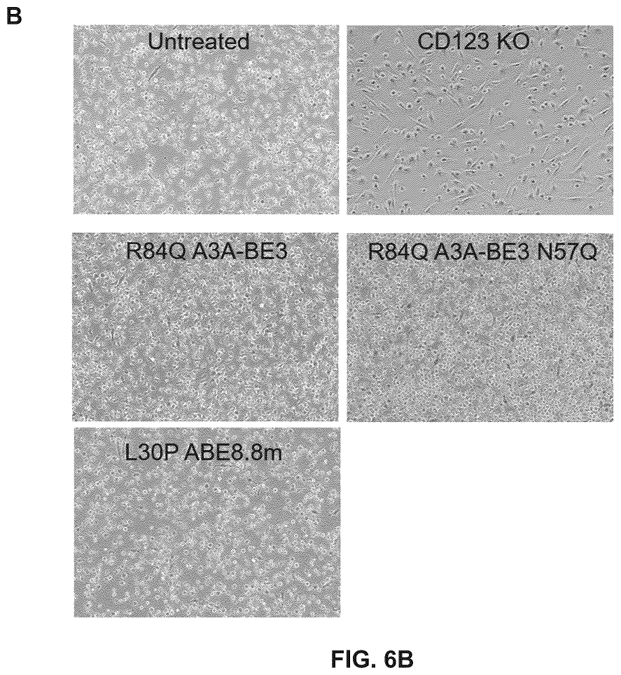

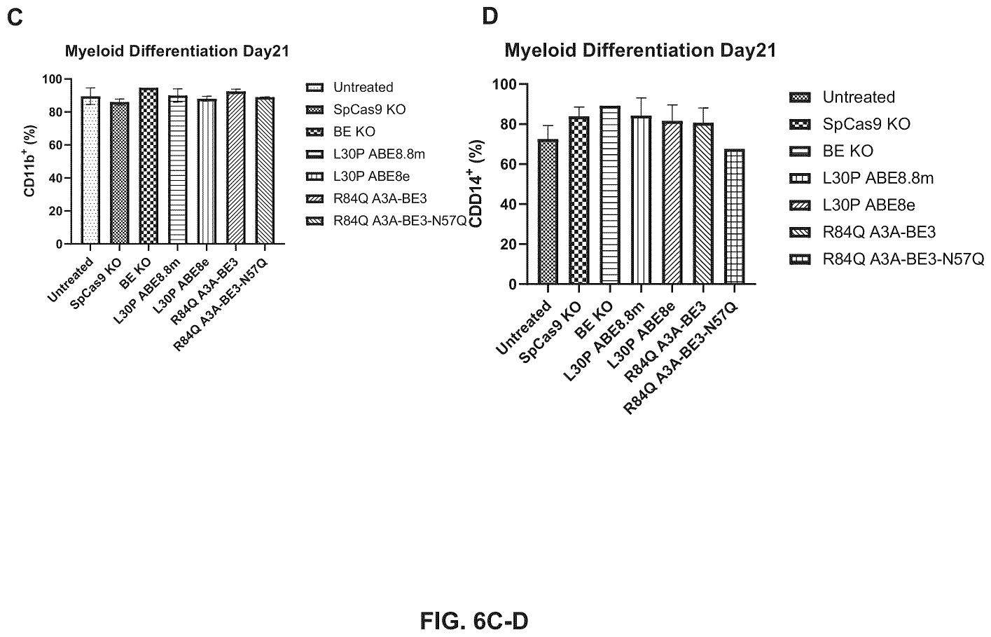

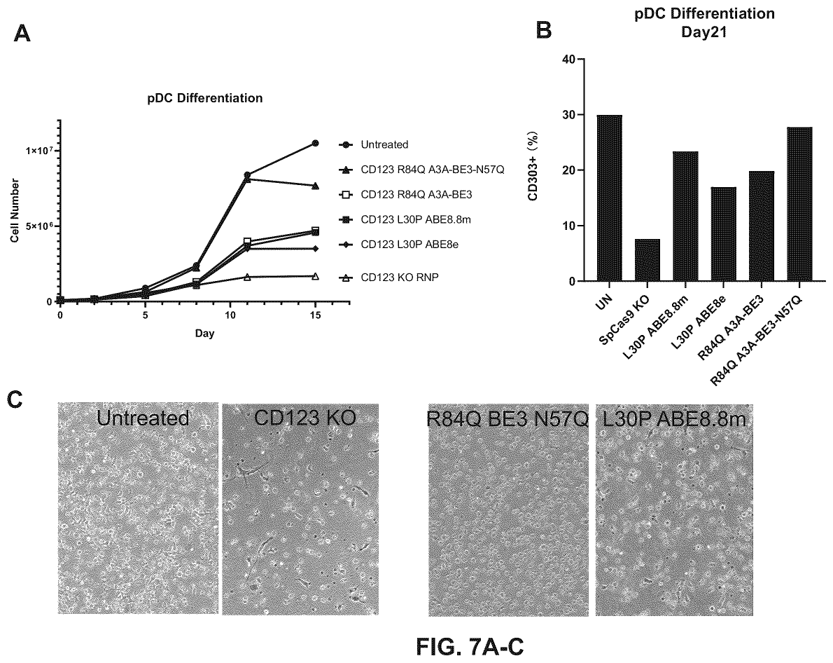

Further validation in hematopoietic stem progenitor cells confirmed that precise editing of the CD123 antigen site does not affect stem cell differentiation and function. Compared to CD123 knockout cells, cells with precise editing of CD123 R84Q or L30P antigen sites showed better cell viability (FIG. 6A), and their cell morphology was consistent with the unedited control (FIG. 6B). Flow cytometry analysis of myeloid differentiation and plasmacytoid dendritic cell differentiation proportions were consistent with the control (FIG. 6C and FIG. 7).

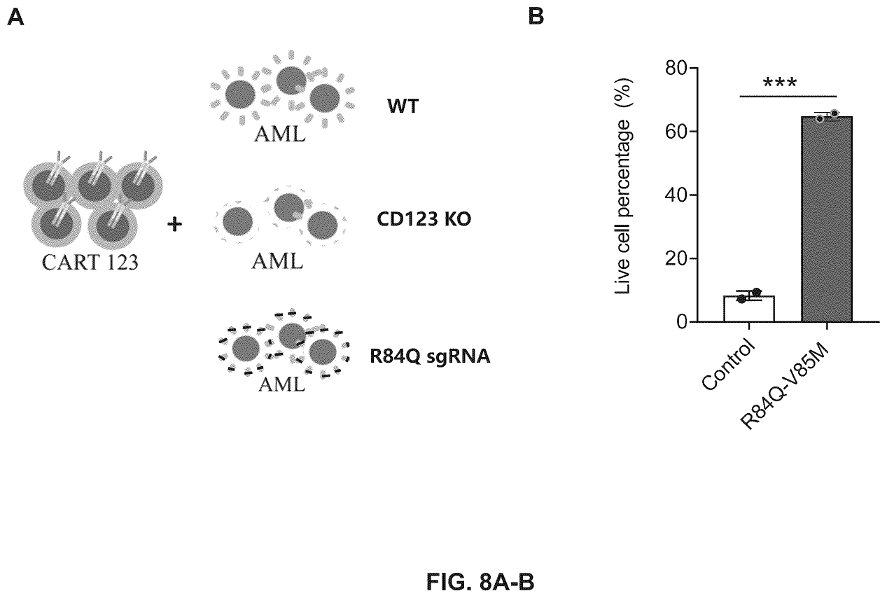

In CD123-CAR-T cell targeting experiments, cells with precise mutations could evade CAR-T cell killing. (A) CAR-T cells containing CSL362 monoclonal antibody were prepared and co-cultured with wild-type AML tumor cells, CD123 knockout AML cells, or cells containing CD123-R84Q mutation. CAR-T efficiently killed wild-type tumor cells expressing CD123, while the killing efficiency was significantly reduced for cells containing CD123-R84Q mutation (FIG. 8A-C).

Additional similar testing was conduced for other epitope residues in CD33 and CD123, as well as for CD117 and CLL-1. The target residues and their corresponding spacer sequences used in gRNA for base editors or pegRNA for prime editors are summarized in Table 1 below.

TABLE 1A
Epitope Residues in CD33

| Antibody | Target Residues in Epitope |
|---|---|
| my9.6 | C41, W60, I105, D112, Y116, F118, P132, W22, G34, |
| | R89, N100, N113, S131 |
| HM195 | C41, W60, 1105, Y116, F118 |

TABLE 1B
CD33 Protein Sequence (SEQ ID NO: 1)

MPLLLLLPLLWAGALAMDPNFWLQVQESVTVQEGLCVLVPCTFFHPIPYY
DKNSPVHGYWFREGAIISRDSPVATNKLDQEVQEETQGRFRLLGDPSRNN
CSLSIVDARRRDNGSYFFRMERGSTKYSYKSPQLSVHVTDLTHRPKILIP
GTLEPGHSKNLTCSVSWACEQGTPPIFSWLSAAPTSLGPRITHSSVLIIT
PRPQDHGTNLTCQVKFAGAGVTTERTIQLNVTYVPQNPTTGIFPGDGSGK
QETRAGVVHGAIGGAGVTALLALCLCLIFFIVKTHRRKAARTAVGRNDTH
PTTGSASPKHQKKSKLHGPTETSSCSGAAPTVEMDEELHYASLNFHGMNP
SKDTSTEYSEVRTQ

TABLE 1C
VH/VL of my9.6

| | | SEQ |
|---|---|---|
| | | ID |
| Region | Sequence | NO: |
| VH | QVQLQQPGAEVVKPGASVKMSCKASGYTFTSYYIHWI | 5 |
| | KQTPGQGLEWVGVIYPGNDDISYNQKFQGKATLTADK | |
| | SSTTAYMQLSSLTSEDSAVYYCAREVRLRYFDVWGQG | |
| | TTVTVSS | |
| VL | EIVLTQSPGSLAVSPGERVTMSCKSSQSVFFSSSQKN | 6 |
| | YLAWYQQIPGQSPRLLIYWASTRESGVPDRFTGSGSG | |
| | TDFTLTISSVQPEDLAIYYCHQYLSSRTFGQGTKLEI | |
| | KR | |

TABLE 1D
VH/VL of HM195

| | | SEQ |
|---|---|---|
| | | ID |
| Region | Sequence | NO: |
| VH | QVQLVQSGAEVKKPGSSVKVSCKASGYTFTDYNMHWVR | 7 |
| | QAPGQGLEWIGYIYPYNGGTGYNQKFKSKATITADEST | |
| | NTAYMELSSLRSEDTAVYYCARGRPAMDYWGQGTLVTV | |
| | SS | |
| VL | DIQMTQSPSSLSASVGDRVTITCRASESVDNYGISFMN | 8 |
| | WFQQKPGKAPKLLIYAASNQGSGVPSRFSGSGSGTDFT | |
| | LTISSLQPDDFATYYCQQSKEVPWTFGQGTKVEIK | |

TABLE 1E
Example spacer sequences of gRNA for editing CD33
epitope

| | | | | SEQ |
|---|---|---|---|---|
| | | | | ID |
| Residue | Editor | Cas | Spacer | NO: |
| C41 | ABE/CBE | Sp cas9 | gtgcagggcacgaggacgca | 19 |
| | | | aagtgcagggcacgaggacg | 20 |
| | | | gaaagtgcagggcacgagga | 21 |
| | CBE | | aagaaagtgcagggcacgag | 22 |
| | | | gaagaaagtgcagggcacga | 23 |
| | | | ggaagaaagtgcagggcacg | 24 |
| | | | tggaagaaagtgcagggcac | 25 |
| | | | tgcagggcacgaggacgcac | 26 |
| | | | atggaagaaagtgcagggca | 27 |
| | | | ggatggaagaaagtgcaggg | 28 |
| | | | tgggatggaagaaagtgcag | 29 |
| | | At cas9 | atggaagaaagtgcagggcacg | 30 |
| | | | ggaagaaagtgcagggcacgag | 31 |
| | | | aagaaagtgcagggcacgagga | 32 |
| | | | gaagaaagtgcagggcacgagg | 33 |
| W60 | ABE | Sp cas9 | cggaaccagtaaccatgaac | 34 |
| | | | ggaaccagtaaccatgaact | 35 |
| | | | gaaccagtaaccatgaactg | 36 |
| | | | accagtaaccatgaactggg | 37 |
| | | | aaccagtaaccatgaactgg | 38 |
| | | | ccagtaaccatgaactgggg | 39 |
| | | | cttcccggaaccagtaacca | 40 |
| | | | tcccggaaccagtaaccatg | 41 |
| | | | ttcccggaaccagtaaccat | 42 |
| | | | tccttcccggaaccagtaac | 43 |
| | | | ggctccttcccggaaccagt | 44 |
| | | At cas9 | aaccagtaaccatgaactgggg | 45 |
| | | | ttcccggaaccagtaaccatga | 46 |
| 1105 | ABE | Sp cas9 | tacgatgctcagggagcagt | 47 |
| | | | gtctacgatgctcagggagc | 48 |
| | | | cgtctacgatgctcagggag | 49 |
| | | | ggcgtctacgatgctcaggg | 50 |
| | | | tggcgtctacgatgctcagg | 51 |
| | | | ctggcgtctacgatgctcag | 52 |
| | | | cctggcgtctacgatgctca | 53 |
| | | | tcctggcgtctacgatgctc | 54 |
| | | | ctcctggcgtctacgatgct | 55 |
| | | At cas9 | tctacgatgctcagggagcagt | 56 |
| | | | ctacgatgctcagggagcagtt | 57 |
| | | | cgatgctcagggagcagttgtt | 58 |
| D112 | ABE | At cas9 | ggagggataatggttcatactt | 59 |
| | | | ggaggagggataatggttcata | 60 |
| | | | aggaggagggataatggttcat | 61 |
| | | Sp cas9 | ataatggttcatacttcttt | 62 |
| | | | gacgccaggaggagggataa | 63 |
| | | | ggaggagggataatggttca | 64 |
| | | | caggaggagggataatggtt | 65 |
| Y116 | ABE | Sp cas9 | ttcatacttctttcggatgg | 66 |
| | | | tcatacttctttcggatgga | 67 |
| | | | gttcatacttctttcggatg | 68 |
| | | | ccgaaagaagtatgaaccat | 69 |
| | | At cas9 | aatggttcatacttctttcgga | 70 |
| | | | ggttcatacttctttcggatgg | 71 |
| | | | catacttctttcggatggagag | 72 |
| F118 | ABE | Sp cas9 | ccgaaagaagtatgaaccat | 73 |
| | | | catccgaaagaagtatgaac | 74 |
| | | | ctctctccatccgaaagaag | 75 |
| | | At cas9 | catccgaaagaagtatgaacca | 76 |
| | | | atccgaaagaagtatgaaccat | 77 |
| | | | tccgaaagaagtatgaaccatt | 78 |
| | | | cgaaagaagtatgaaccattat | 79 |
| | | | ctctccatccgaaagaagtatg | 80 |
| P132 | CBE | Sp cas9 | caaatctccccagctctctg | 81 |
| | | | tctccccagctctctgtgca | 82 |
| | | | aatctccccagctctctgtg | 83 |
| | | | tacaaatctccccagctctc | 84 |
| | | At cas9 | atctccccagctctctgtgcat | 85 |
| | | | gttacaaatctccccagctctc | 86 |
| | | | atacagttacaaatctccccag | 87 |
| | | | aaatctccccagctctctgtgc | 88 |
| | | | acaaatctccccagctctctgt | 89 |
| W22 | ABE | Sp cas9 | agaaatttggatccatagcc | 90 |
| | | | tgcacttgcagccagaaatt | 91 |
| | | | agccagaaatttggatccat | 92 |
| | | | cagccagaaatttggatcca | 93 |
| | | | tgcagccagaaatttggatc | 94 |
| | | | cacttgcagccagaaatttg | 95 |
| | | | gcacttgcagccagaaattt | 96 |
| | | At cas9 | tgcacttgcagccagaaatttg | 97 |
| | | | tgcagccagaaatttggatcca | 98 |
| | | | ccagaaatttggatccatagcc | 99 |
| | | | cttgcagccagaaatttggatc | 100 |
| G34 | ABE | Sp cas9 | aaaccctcctgtaccgtcac | 101 |
| | | | cacaaaccctcctgtaccgt | 102 |
| | | | acgcacaaaccctcctgtac | 103 |
| | | | aggacgcacaaaccctcctg | 104 |
| | | | cgaggacgcacaaaccctcc | 105 |
| | CBE | At cas9 | gacgcacaaaccctcctgtacc | 106 |
| | | | aaaccctcctgtaccgtcactg | 107 |
| | | | caaaccctcctgtaccgtcact | 108 |
| | | | cacaaaccctcctgtaccgtca | 109 |
| | | | cgaggacgcacaaaccctcctg | 110 |
| R89 | CBE | Sp cas9 | aggaggcggaatctgccctg | 111 |
| | | | aaggaggcggaatctgccct | 112 |
| | | At cas9 | aggcggaatctgccctgagtct | 113 |
| | | | gaatctgccctgagtctcctcc | 114 |
| | | | gaggcggaatctgccctgagtc | 115 |
| | | | aggaggcggaatctgccctgag | 116 |
| | | | aaggaggcggaatctgccctga | 117 |
| | | | ccaaggaggcggaatctgccct | 118 |
| N100 | ABE/CBE | Sp cas9 | aacaactgctccctgagcat | 119 |
| | | | aggaacaactgctccctgag | 120 |
| | | | gtaggaacaactgctccctg | 121 |
| | | | agtaggaacaactgctccct | 122 |
| | | | cagtaggaacaactgctccc | 123 |
| | | At cas9 | gggatcccagtaggaacaactg | 124 |
| | | | agtaggaacaactgctccctga | 125 |
| | | | cccagtaggaacaactgctccc | 126 |
| | | | ggggatcccagtaggaacaact | 127 |
| | | | atcccagtaggaacaactgctc | 128 |
| N113 | ABE | Sp cas9 | ataatggttcatacttcttt | 129 |
| | | | ggaggagggataatggttca | 130 |
| | | At cas9 | ggagggataatggttcatactt | 131 |
| | | | aggaggagggataatggttcat | 132 |
| | | | ataatggttcatacttctttcg | 133 |
| S131 | ABE | Sp cas9 | gggagatttgtaactgtatt | 134 |
| | | | gagctggggagatttgtaac | 135 |
| | | | gctggggagatttgtaactg | 136 |
| | CBE | | tctccccagctctctgtgca | 137 |
| | | | caaatctccccagctctctg | 138 |
| | | | tacaaatctccccagctctc | 139 |
| | | | aatctccccagctctctgtg | 140 |
| | CBE | At cas9 | atctccccagctctctgtgcat | 141 |
| | | | acaaatctccccagctctctgt | 142 |
| | | | aaatctccccagctctctgtgc | 143 |
| | ABE | | agctggggagatttgtaactgt | 144 |

TABLE IF
Example spacer sequences of pegRNA for editing
CD33 epitope

| | | SEQ |
|---|---|---|
| | | ID |
| Residue | Spacer | NO: |
| W22, G34, C41 | TGGCTATGGATCCAAATTTCTGG | 145 |
| | AAATTTCTGGCTGCAAGTGCAGG | 146 |
| | GCAGGAGTCAGTGACGGTACAGG | 147 |
| | GGAGTCAGTGACGGTACAGGAGG | 148 |
| | GAGTCAGTGACGGTACAGGAGGG | 149 |
| | GGGAGAGGGGTTGTCGGGCTGGG | 150 |
| | GTGGGCAGGTGAGTGGCTGTGGG | 151 |
| | GGGGAGAGGGGTTGTCGGGCTGG | 152 |
| | TCGTTTCCCCACAGGGGCCCTGG | 153 |
| | CTGTGGGGAGAGGGGTTGTCGGG | 154 |
| | CCCCACAGGGGCCCTGGCTATGG | 155 |
| | GCAAGTGCAGGAGTCAGTGACGG | 156 |
| | GCTGACCCTCGTTTCCCCACAGG | 157 |
| | TGACCCTCGTTTCCCCACAGGGG | 158 |
| | CTGACCCTCGTTTCCCCACAGGG | 159 |
| | TGTGGGCAGGTGAGTGGCTGTGG | 160 |
| | TGGGCAGGTGAGTGGCTGTGGGG | 161 |
| | GCTGTGGGGAGAGGGGTTGTCGG | 162 |
| | CCCTGCTGTGGGCAGGTGAGTGG | 163 |
| | GTGAGTGGCTGTGGGGAGAGGGG | 164 |
| | GGTGAGTGGCTGTGGGGAGAGGG | 165 |
| | AGGTGAGTGGCTGTGGGGAGAGG | 166 |
| | GGGGAGTTCTTGTCGTAGTAGGG | 167 |
| | TGGGGAGTTCTTGTCGTAGTAGG | 168 |
| | GTTCTTGTCGTAGTAGGGTATGG | 169 |
| | TTCTTGTCGTAGTAGGGTATGGG | 170 |
| | TGGAGAGTCCCTGGATATAATGG | 171 |
| | GAACCAGTAACCATGAACTGGGG | 172 |
| | TGTCGTAGTAGGGTATGGGATGG | 173 |
| | TGGATATAATGGCTCCTTCCCGG | 174 |
| | TGTGGCCACTGGAGAGTCCCTGG | 175 |
| | CGGAACCAGTAACCATGAACTGG | 176 |
| | GGAAGAAAGTGCAGGGCACGAGG | 177 |
| | ATGGGATGGAAGAAAGTGCAGGG | 178 |
| | TATGGGATGGAAGAAAGTGCAGG | 179 |
| | GGAACCAGTAACCATGAACTGGG | 180 |
| W60 | GACAAGAACTCCCCAGTTCATGG | 181 |
| | TGGAGAGTCCCTGGATATAATGG | 182 |
| | TGGATATAATGGCTCCTTCCCGG | 183 |
| | TCTAGCTTGTTTGTGGCCACTGG | 184 |
| | TTCTTGATCTAGCTTGTTTGTGG | 185 |
| | TGTGGCCACTGGAGAGTCCCTGG | 186 |
| R89, N100, 1105 | GGGAAGGAGCCATTATATCCAGG | 187 |
| | GCCTCCTTGGGGATCCCAGTAGG | 188 |
| | GGAAGGAGCCATTATATCCAGGG | 189 |
| | GCTAGATCAAGAAGTACAGGAGG | 190 |
| | TATATCCAGGGACTCTCCAGTGG | 191 |
| | GAAGTACAGGAGGAGACTCAGGG | 192 |
| | AGAAGTACAGGAGGAGACTCAGG | 193 |
| | CATGGTTACTGGTTCCGGGAAGG | 194 |
| | CAAGCTAGATCAAGAAGTACAGG | 195 |
| | AGGGCAGATTCCGCCTCCTTGGG | 196 |
| | CAGTTCATGGTTACTGGTTCCGG | 197 |
| | GGGCAGATTCCGCCTCCTTGGGG | 198 |
| | AGTTCATGGTTACTGGTTCCGGG | 199 |
| | CAGGGCAGATTCCGCCTCCTTGG | 200 |
| | AGGGAGCAGTTGTTCCTACTGGG | 201 |
| | GGGAGATTTGTAACTGTATTTGG | 202 |
| | CAGGGAGCAGTTGTTCCTACTGG | 203 |
| | TGTTCCTACTGGGATCCCCAAGG | 204 |
| | TGAACCATTATCCCTCCTCCTGG | 205 |
| | TCCTACTGGGATCCCCAAGGAGG | 206 |
| | TCCTGGCGTCTACGATGCTCAGG | 207 |
| | TACTGGGATCCCCAAGGAGGCGG | 208 |
| | CCTGGCGTCTACGATGCTCAGGG | 209 |
| | TGTCACATGCACAGAGAGCTGGG | 210 |
| | GTCACATGCACAGAGAGCTGGGG | 211 |
| | CTGTCACATGCACAGAGAGCTGG | 212 |
| D112, N113, Y116, F118 | GGGAGATTTGTAACTGTATTTGG | 213 |
| | TGTCACATGCACAGAGAGCTGGG | 214 |
| | GTCACATGCACAGAGAGCTGGGG | 215 |
| | CTGTCACATGCACAGAGAGCTGG | 216 |
| S131, P132 | TGGTTCATACTTCTTTCGGATGG | 217 |
| | GACGCCAGGAGGAGGGATAATGG | 218 |
| | GCATCGTAGACGCCAGGAGGAGG | 219 |
| | CCCTGAGCATCGTAGACGCCAGG | 220 |
| | CATCGTAGACGCCAGGAGGAGGG | 221 |
| | TGAGCATCGTAGACGCCAGGAGG | 222 |
| | ATAATGGTTCATACTTCTTTCGG | 223 |
| | TACTTCTTTCGGATGGAGAGAGG | 224 |
| | GTACCCATGAACTTCCCTTGCGG | 225 |
| | TGTCACATGCACAGAGAGCTGGG | 226 |
| | GTCACATGCACAGAGAGCTGGGG | 227 |
| | CTGTCACATGCACAGAGAGCTGG | 228 |

TABLE 2A
Epitope Residues in CD123

| Antibody | Target Residues in Epitope |
|---|---|
| CSL362 or 32716 | I27, L30, M32, W41, E51, C52, |
| | S59, P61, R84, P88, F90, S91, W93 |

TABLE 2B
CD123 Protein Sequence (SEQ ID NO: 2)

MVLLWLTLLLIALPCLLQTKEDPNPPITNLRMKAKAQQLTWDLNRNVTDI
ECVKDADYSMPAVNNSYCQFGAISLCEVTNYTVRVANPPFSTWILFPENS
GKPWAGAENLTCWIHDVDFLSCSWAVGPGAPADVQYDLYLNVANRRQQYE
CLHYKTDAQGTRIGCRFDDISRLSSGSQSSHILVRGRSAAFGIPCTDKFV
VFSQIEILTPPNMTAKCNKTHSFMHWKMRSHFNRKFRYELQIQKRMQPVI
TEQVRDRTSFQLLNPGTYTVQIRARERVYEFLSAWSTPQRFECDQEEGAN
TRAWRTSLLIALGTLLALVCVFVICRRYLVMQRLFPRIPHMKDPIGDSFQ
NDKLVVWEAGKAGLEECLVTEVQVVQKT

TABLE 2C
VH/VL of CSL362

| Region | Sequence | SEQ ID NO: |
|---|---|---|
| VH | EVQLVQSGAEVKKPGESLKISCKGSGYS | 9 |
| | FTDYYMKWARQMPGKGLEWMGDIIPSNG | |
| | ATFYNQKFKGQVTISADKSISTTYLQWS | |
| | SLKASDTAMYYCARSHLLRASWFAYWGQ | |
| | GTMVTVSSASTKGPSVFPLAPSSKSTSG | |
| | GTAALGCLVKDYFPEPVTVSWNSGALTS | |
| | GVHTFPAVLQSS | |
| VL | EVQLVQSGAEVKKPGESLKISCKGSGYS | 10 |
| | FTDYYMKWARQMPGKGLEWMGDIIPSNG | |
| | ATFYNQKFKGQVTISADKSISTTYLQWS | |
| | SLKASDTAMYYCARSHLLRASWFAYWGQ | |
| | GTMVTVSSASTKGPSVFPLAPSSKSTSG | |
| | GTAALGCLVKDYFPEPVTVSWNSGALTS | |
| | GVHTFPAVLQSS | |

TABLE 2D
VH/VL of 32716

| Region | Sequence | SEQ ID NO: |
|---|---|---|
| VH | QIQLVQSGPELKKPGETVKISCKASGYI | 11 |
| | FTNYGMNWVKQAPGKSFKWMGWINTYTG | |
| | ESTYSADFKGRFAFSLETSASTAYLHIN | |
| | DLKNEDTATYFCARSGGYDPMDYWGQGT | |
| | SVTVSS | |
| VL | DIVLTQSPASLAVSLGQRATISCRASES | 12 |
| | VDNYGNTFMHWYQQKPGQPPKLLIYRAS | |
| | NLESGIPARFSGSGSRTDFTLTINPVEA | |
| | DDVATYYCQQSNEDPPTFGAGTKLELK | |

TABLE 2E
Example spacer sequences of
gRNA for editing CD123 epitope

| | | | | SEQ |
|---|---|---|---|---|
| Res- | Ed- | | | ID |
| idue | itor | Cas | Spacer | NO: |
| I27 | ABE | SpCas9 | AGGTTCGTGATTGGTGGGTT | 229 |
| | | | TCCTTAGGTTCGTGATTGGT | 230 |
| | | | TTCATCCTTAGGTTCGTGAT | 231 |
| | | | GGTTCGTGATTGGTGGGTTT | 232 |
| | | | CCTTAGGTTCGTGATTGGTG | 233 |
| | | | TCATCCTTAGGTTCGTGATT | 234 |
| | | | GTTCGTGATTGGTGGGTTTG | 235 |
| | | AtCas9 | GTGATTGGTGGGTTTGGATCTT | 236 |
| | | | TTCGTGATTGGTGGGTTTGGAT | 237 |
| | | | GTTCGTGATTGGTGGGTTTGGA | 238 |
| | | | TAGGTTCGTGATTGGTGGGTTT | 239 |
| | | Cpf1 | ATCCTTAGGTTCGTGATTGG | 240 |
| | ABE | SpCas9 | ACCCACCAATCACGAACCTA | 241 |
| | | | ATCACGAACCTAAGGATGAA | 242 |
| | | | ACCAATCACGAACCTAAGGA | 243 |
| | | | CCCACCAATCACGAACCTAA | 244 |
| | | | AGATCCAAACCCACCAATCA | 245 |
| | | | AATCACGAACCTAAGGATGA | 246 |
| | | | CAATCACGAACCTAAGGATG | 247 |
| | | | CCAATCACGAACCTAAGGAT | 248 |
| | | | CCACCAATCACGAACCTAAG | 249 |
| | | | AACCCACCAATCACGAACCT | 250 |
| | | | AAACCCACCAATCACGAACC | 251 |
| | | | ATCCAAACCCACCAATCACG | 252 |
| | | | GATCCAAACCCACCAATCAC | 253 |
| | | | GAAGATCCAAACCCACCAAT | 254 |
| | | AtCas9 | ACCAATCACGAACCTAAGGATG | 255 |
| | | | AAGATCCAAACCCACCAATCAC | 256 |
| | | | GAAGATCCAAACCCACCAATCA | 257 |
| | | | CCACCAATCACGAACCTAAGGA | 258 |
| | | | CAAACCCACCAATCACGAACCT | 259 |
| | | | ATCCAAACCCACCAATCACGAA | 260 |
| | | | CACCAATCACGAACCTAAGGAT | 261 |
| | | | AACCCACCAATCACGAACCTAA | 262 |
| | | Cpf1 | GATCCAAACCCACCAATCAC | 263 |
| L30 | ABE | SpCas9 | AGGTTCGTGATTGGTGGGTT | 264 |
| | | | TCCTTAGGTTCGTGATTGGT | 265 |
| | | | ATCCTTAGGTTCGTGATTGG | 266 |
| | | | TTCATCCTTAGGTTCGTGAT | 267 |
| | | | CCTTAGGTTCGTGATTGGTG | 268 |
| | | | TCATCCTTAGGTTCGTGATT | 269 |
| | | | TGCTTTCATCCTTAGGTTCG | 270 |
| | | | TTTGCTTTCATCCTTAGGTT | 271 |
| | | | AGCCTTTGCTTTCATCCTTA | 272 |
| | | | GCTTTCATCCTTAGGTTCGT | 273 |
| | | AtCas9 | TAGGTTCGTGATTGGTGGGTTT | 274 |
| | | | TGAGCCTTTGCTTTCATCCTTA | 275 |
| | | Cpf1 | ATCCTTAGGTTCGTGATTGG | 276 |
| | CBE | SpCas9 | GAACCTAAGGATGAAAGCAA | 277 |
| | | | ACCCACCAATCACGAACCTA | 278 |
| | | | AACCTAAGGATGAAAGCAAA | 279 |
| | | | ATCACGAACCTAAGGATGAA | 280 |
| | | | ACCAATCACGAACCTAAGGA | 281 |
| | | | CCCACCAATCACGAACCTAA | 282 |
| | | | CGAACCTAAGGATGAAAGCA | 283 |
| | | | ACGAACCTAAGGATGAAAGC | 284 |
| | | | CACGAACCTAAGGATGAAAG | 285 |
| | | | AATCACGAACCTAAGGATGA | 286 |
| | | | CAATCACGAACCTAAGGATG | 287 |
| | | | CCACCAATCACGAACCTAAG | 288 |
| | | | AACCCACCAATCACGAACCT | 289 |
| | | | AAACCCACCAATCACGAACC | 290 |
| | | AtCas9 | CACGAACCTAAGGATGAAAGCA | 291 |
| | | | ACCAATCACGAACCTAAGGATG | 292 |
| | | | ACCTAAGGATGAAAGCAAAGGC | 293 |
| | | | CGAACCTAAGGATGAAAGCAAA | 294 |
| | | | GAACCTAAGGATGAAAGCAAAG | 295 |
| | | | CAAACCCACCAATCACGAACCT | 296 |
| | | | AACCCACCAATCACGAACCTAA | 297 |
| | | | CACCAATCACGAACCTAAGGAT | 298 |
| M32 | ABE | SpCas9 | TTCATCCTTAGGTTCGTGAT | 299 |
| | | | GAGCCTTTGCTTTCATCCTT | 300 |
| | | | TGCTTTCATCCTTAGGTTCG | 301 |
| | | | TTTGCTTTCATCCTTAGGTT | 302 |
| | | | AGCCTTTGCTTTCATCCTTA | 303 |
| | | | GCTTTCATCCTTAGGTTCGT | 304 |
| | | | TGAGCCTTTGCTTTCATCCT | 305 |
| | | AtCas9 | TGAGCCTTTGCTTTCATCCTTA | 306 |
| | | | GAGCCTTTGCTTTCATCCTTAG | 307 |
| | | Cpf1 | CTTTCATCCTTAGGTTCGTG | 308 |
| | ABE | SpCas9 | GAACCTAAGGATGAAAGCAA | 309 |
| | | | GATGAAAGCAAAGGCTCAGC | 310 |
| | | | AAGGATGAAAGCAAAGGCTC | 311 |
| | | | AACCTAAGGATGAAAGCAAA | 312 |
| | | | ATCACGAACCTAAGGATGAA | 313 |
| | | | ACCAATCACGAACCTAAGGA | 314 |
| | | | GGATGAAAGCAAAGGCTCAG | 315 |
| | | | TAAGGATGAAAGCAAAGGCT | 316 |
| | | | CGAACCTAAGGATGAAAGCA | 317 |
| | | | ACGAACCTAAGGATGAAAGC | 318 |
| | | | AATCACGAACCTAAGGATGA | 319 |
| | | | CAATCACGAACCTAAGGATG | 320 |
| | | | CCAATCACGAACCTAAGGAT | 321 |
| | | AtCas9 | CACGAACCTAAGGATGAAAGCA | 322 |
| | | | ACCAATCACGAACCTAAGGATG | 323 |
| | | | ATGAAAGCAAAGGCTCAGCAGT | 324 |
| | | | GATGAAAGCAAAGGCTCAGCAG | 325 |
| | | | ACCTAAGGATGAAAGCAAAGGC | 326 |
| | | | CGAACCTAAGGATGAAAGCAAA | 327 |
| | | | GAACCTAAGGATGAAAGCAAAG | 328 |
| | | | CCACCAATCACGAACCTAAGGA | 329 |
| | | | CACCAATCACGAACCTAAGGAT | 330 |
| W41 | ABE | SpCas9 | TAAGGTCCCAGGTCAACTGC | 331 |
| | | | CAGGTCAACTGCTGAGCCTT | 332 |
| | | | AGGTCCCAGGTCAACTGCTG | 333 |
| | | | TGTTAAGGTCCCAGGTCAAC | 334 |
| | | | ACATTTCTGTTAAGGTCCCA | 335 |
| | | | AAGGTCCCAGGTCAACTGCT | 336 |
| | | | TTCTGTTAAGGTCCCAGGTC | 337 |
| | | AtCas9 | TTTCTGTTAAGGTCCCAGGTCA | 338 |
| | | | CAGGTCAACTGCTGAGCCTTTG | 339 |
| | | | GTCCCAGGTCAACTGCTGAGCC | 340 |
| | | | TTAAGGTCCCAGGTCAACTGCT | 341 |
| | | | GTTAAGGTCCCAGGTCAACTGC | 342 |
| | | | TTTCTGTTAAGGTCCCAGGTCA | 343 |
| | | | ACATTTCTGTTAAGGTCCCAGG | 344 |
| | | Cpf1 | TGTTAAGGTCCCAGGTCAAC | 345 |
| E51 | CBE | SpCas9 | GTCTTTAACACACTCGATAT | 346 |
| | | | CTCGATATCGGTCACATTTC | 347 |
| | | | AACACACTCGATATCGGTCA | 348 |
| | | | TTAACACACTCGATATCGGT | 349 |
| | | | GGCGTCTTTAACACACTCGA | 350 |
| | | | TCGGCGTCTTTAACACACTC | 351 |
| | | AtCas9 | GCGTCTTTAACACACTCGATAT | 352 |
| | | | TAACACACTCGATATCGGTCAC | 353 |
| | | | GTCTTTAACACACTCGATATCG | 354 |
| | | | GTCGGCGTCTTTAACACACTCG | 355 |
| | | | ACACACTCGATATCGGTCACAT | 356 |
| | | | ACACACTCGATATCGGTCACAT | 357 |
| | | Cpf1 | ACACACTCGATATCGGTCAC | 358 |
| | ABE | SpG | ATCGAGTGTGTTAAAGACGC | 359 |
| | | | GATATCGAGTGTGTTAAAGA | 360 |
| | | | ACCGATATCGAGTGTGTTAA | 361 |
| | | | AATGTGACCGATATCGAGTG | 362 |
| | | | GAAATGTGACCGATATCGAG | 363 |
| | | | AGTGTGTTAAAGACGCCGAC | 364 |
| | | | TCGAGTGTGTTAAAGACGCC | 365 |
| | | | CCGATATCGAGTGTGTTAAA | 366 |
| | | | GACCGATATCGAGTGTGTTA | 367 |
| | | | TGACCGATATCGAGTGTGTT | 368 |
| | | AtCas9 | GACCGATATCGAGTGTGTTAAA | 369 |
| | | | CGAGTGTGTTAAAGACGCCGAC | 370 |
| | | | GATATCGAGTGTGTTAAAGACG | 371 |
| | | | ACCGATATCGAGTGTGTTAAAG | 372 |
| | | | GTGACCGATATCGAGTGTGTTA | 373 |
| | | | CGATATCGAGTGTGTTAAAGAC | 374 |
| | | | AAATGTGACCGATATCGAGTGT | 375 |
| | | | ACAGAAATGTGACCGATATCGA | 376 |
| C52 | CBE | SpCas9 | GTCTTTAACACACTCGATAT | 377 |
| | ABE | | TCTTTAACACACTCGATATC | 378 |
| | | | GTCGGCGTCTTTAACACACT | 379 |
| | | | AACACACTCGATATCGGTCA | 380 |
| | | | TTAACACACTCGATATCGGT | 381 |
| | | | GGCGTCTTTAACACACTCGA | 382 |
| | | | TCGGCGTCTTTAACACACTC | 383 |
| | | AtCas9 | GCGTCTTTAACACACTCGATAT | 384 |
| | | | TAACACACTCGATATCGGTCAC | 385 |
| | | | GTCTTTAACACACTCGATATCG | 386 |
| | | | GTCGGCGTCTTTAACACACTCG | 387 |
| | | | ACACACTCGATATCGGTCACAT | 388 |
| | | | CGGCGTCTTTAACACACTCGAT | 389 |
| | | | GAATAGTCGGCGTCTTTAACAC | 390 |
| | | Cpf1 | ACACACTCGATATCGGTCAC | 391 |
| S59 | CBE | SpCas9 | ATTTACCGGCATAGAATAGT | 392 |
| | | | GACGCCGACTATTCTATGCC | 393 |
| | | | TAAAGACGCCGACTATTCTA | 394 |
| | | | TATTCTATGCCGGTAAATCA | 395 |
| | | | ACTATTCTATGCCGGTAAAT | 396 |
| | | | CCGACTATTCTATGCCGGTA | 397 |
| | | | GCCGACTATTCTATGCCGGT | 398 |
| | | AtCas9 | ACGCCGACTATTCTATGCCGGT | 399 |
| | | | TATTCTATGCCGGTAAATCATA | 400 |
| | | | ACTATTCTATGCCGGTAAATCA | 401 |
| | | | CGACTATTCTATGCCGGTAAAT | 402 |
| | | | TGTTAAAGACGCCGACTATTCT | 403 |
| | | | AGACGCCGACTATTCTATGCCG | 404 |
| | | | TTAAAGACGCCGACTATTCTAT | 405 |
| | | | GTTAAAGACGCCGACTATTCTA | 406 |
| | ABE | SpCas9 | ATTTACCGGCATAGAATAGT | 407 |
| | | | TTTACCGGCATAGAATAGTC | 408 |
| | | | ATGATTTACCGGCATAGAAT | 409 |
| | | | AATAGTCGGCGTCTTTAACA | 410 |
| | | | AGAATAGTCGGCGTCTTTAA | 411 |
| | | | ATAGAATAGTCGGCGTCTTT | 412 |
| | | AtCas9 | CGGCATAGAATAGTCGGCGTCT | 413 |
| | | | AGAATAGTCGGCGTCTTTAACA | 414 |
| | | | ATAGAATAGTCGGCGTCTTTAA | 415 |
| | | | GCATAGAATAGTCGGCGTCTTT | 416 |
| | | | CGGCATAGAATAGTCGGCGTCT | 417 |
| | | | ATTTACCGGCATAGAATAGTCG | 418 |
| | | | ACCGGCATAGAATAGTCGGCGT | 419 |
| | | | GAATAGTCGGCGTCTTTAACAC | 420 |
| R84 | CBE | SpCas9 | GGCCACTCGGACGGTGTAGT | 421 |
| | | | TGGTGGGTTGGCCACTCGGA | 422 |
| | | | GTTGGCCACTCGGACGGTGT | 423 |
| | | | TGGGTTGGCCACTCGGACGG | 424 |
| | | | GGTGGGTTGGCCACTCGGAC | 425 |
| | | | GAATGGTGGGTTGGCCACTC | 426 |
| | | | GCCACTCGGACGGTGTAGTT | 427 |
| | | | ACTCGGACGGTGTAGTTGGT | 428 |
| | | | AATGGTGGGTTGGCCACTCG | 429 |
| | | AtCas9 | CTCGGACGGTGTAGTTGGTCAC | 430 |
| | | | CACTCGGACGGTGTAGTTGGTC | 431 |
| | | | GGCCACTCGGACGGTGTAGTTG | 432 |
| | | | TTGGCCACTCGGACGGTGTAGT | 433 |
| | | | TCGGACGGTGTAGTTGGTCACT | 434 |
| | | | GGTTGGCCACTCGGACGGTGTA | 435 |
| P88 | CBE | SpCas9 | CCAACCCACCATTCTCCACG | 436 |
| | | | CAACCCACCATTCTCCACGT | 437 |
| | | | GGCCAACCCACCATTCTCCA | 438 |
| | | | AACCCACCATTCTCCACGTG | 439 |
| | | | GTGGCCAACCCACCATTCTC | 440 |
| | | | CGTCCGAGTGGCCAACCCAC | 441 |
| | | | CACCGTCCGAGTGGCCAACC | 442 |
| | | AtCas9 | CCACCATTCTCCACGTGGATCC | 443 |
| | | | AACCCACCATTCTCCACGTGGA | 444 |
| | | | CCAACCCACCATTCTCCACGTG | 445 |
| | | | GCCAACCCACCATTCTCCACGT | 446 |
| | | | CCGAGTGGCCAACCCACCATTC | 447 |
| | | | GTCCGAGTGGCCAACCCACCAT | 448 |
| | | | GTCCGAGTGGCCAACCCACCAT | 449 |
| | | | GTCCGAGTGGCCAACCCACCAT | 450 |
| F90 | ABE | SpCas9 | CCACGTGGAGAATGGTGGGT | 451 |
| | | | AGAATGGTGGGTTGGCCACT | 452 |
| | | | GGATCCACGTGGAGAATGGT | 453 |
| | | | AAGAGGATCCACGTGGAGAA | 454 |
| | | | GAATGGTGGGTTGGCCACTC | 455 |
| | | | CACGTGGAGAATGGTGGGTT | 456 |
| | | | GATCCACGTGGAGAATGGTG | 457 |
| | | | AGAGGATCCACGTGGAGAAT | 458 |
| | | | AATGGTGGGTTGGCCACTCG | 459 |
| | | | GTGGAGAATGGTGGGTTGGC | 460 |
| | | AtCas9 | ACGTGGAGAATGGTGGGTTGGC | 461 |
| | | | GGAGAATGGTGGGTTGGCCACT | 462 |
| | | | CCACGTGGAGAATGGTGGGTTG | 463 |
| | | | ATCCACGTGGAGAATGGTGGGT | 464 |
| | | | GATCCACGTGGAGAATGGTGGG | 465 |
| | | | GGATCCACGTGGAGAATGGTGG | 466 |
| S91 | ABE | SpCas9 | CCACGTGGAGAATGGTGGGT | 467 |
| | | | GGATCCACGTGGAGAATGGT | 468 |
| | | | AAGAGGATCCACGTGGAGAA | 469 |
| | | | AGGATCCACGTGGAGAATGG | 470 |
| | | | CACGTGGAGAATGGTGGGTT | 471 |
| | | | GATCCACGTGGAGAATGGTG | 472 |
| | | | AGAGGATCCACGTGGAGAAT | 473 |
| | | | GTGGAGAATGGTGGGTTGGC | 474 |
| | | | GGGAAGAGGATCCACGTGGA | 475 |
| | | AtCas9 | ACGTGGAGAATGGTGGGTTGGC | 476 |
| | | | GGAGAATGGTGGGTTGGCCACT | 477 |
| | | | CCACGTGGAGAATGGTGGGTTG | 478 |
| | | | ATCCACGTGGAGAATGGTGGGT | 479 |
| | | | GATCCACGTGGAGAATGGTGGG | 480 |
| | | | GGATCCACGTGGAGAATGGTGG | 481 |
| | CBE | SpCas9 | CCAACCCACCATTCTCCACG | 482 |
| | | | TCCACGTGGATCCTCTTCCC | 483 |
| | | | CCACGTGGATCCTCTTCCCT | 484 |
| | | | AACCCACCATTCTCCACGTG | 485 |
| | | | GTGGCCAACCCACCATTCTC | 486 |
| | | AtCas9 | ACCATTCTCCACGTGGATCCTC | 487 |
| | | | TCCACGTGGATCCTCTTCCCTG | 488 |
| | | | ACCATTCTCCACGTGGATCCTC | 489 |
| | | | CACCATTCTCCACGTGGATCCT | 490 |
| | | | CCACCATTCTCCACGTGGATCC | 491 |
| | | | AACCCACCATTCTCCACGTGGA | 492 |
| | | | CCAACCCACCATTCTCCACGTG | 493 |
| | | | GCCAACCCACCATTCTCCACGT | 494 |
| | | | TCTCCACGTGGATCCTCTTCCC | 495 |
| | | | ATTCTCCACGTGGATCCTCTTC | 496 |
| | | | CATTCTCCACGTGGATCCTCTT | 497 |
| W93 | ABE | SpCas9 | CCACGTGGAGAATGGTGGGT | 498 |
| | | | GGATCCACGTGGAGAATGGT | 499 |
| | | | AAGAGGATCCACGTGGAGAA | 500 |
| | | | CTCAGGGAAGAGGATCCACG | 501 |
| | | | CACGTGGAGAATGGTGGGTT | 502 |
| | | | GATCCACGTGGAGAATGGTG | 503 |
| | | | AGAGGATCCACGTGGAGAAT | 504 |
| | | | TCAGGGAAGAGGATCCACGT | 505 |
| | | | TTCTCAGGGAAGAGGATCCA | 506 |
| | | AtCas9 | GGAAGAGGATCCACGTGGAG | 507 |
| | | | GGGAAGAGGATCCACGTGGA | 508 |
| | | | CAGGGAAGAGGATCCACGTG | 509 |
| | | | ACGTGGAGAATGGTGGGTTGGC | 510 |
| | | | CCACGTGGAGAATGGTGGGTTG | 511 |
| | | | ATCCACGTGGAGAATGGTGGGT | 512 |
| | | | GATCCACGTGGAGAATGGTGGG | 513 |
| | | | GTTCTCAGGGAAGAGGATCCAC | 514 |
| | | | GGATCCACGTGGAGAATGGTGG | 515 |
| | | | TGTTCTCAGGGAAGAGGATCCA | 516 |

TABLE 2F
Example spacer sequences of
pegRNA for editing CD123 epitope

| Residue | Spacer | SEQ ID NO: |
|---|---|---|
| R84, P88, F90, | AACAATAGCTATTGCCAGTT | 517 |
| S91, W93 | CATAGTCCTATGTCTCTCTT | 518 |
| | AAGACACAGCGAAGGCGAGA | 519 |
| | AAAGACACAGCGAAGGCGAG | 520 |
| | ACACAGCGAAGGCGAGAGGG | 521 |
| | TCTCACTGTTCTCAGGGAAG | 522 |
| | CACAGCGAAGGCGAGAGGGA | 523 |
| | ACATTTTTCTCACTGTTCTC | 524 |
| | CATTTTTCTCACTGTTCTCA | 525 |
| | AAAGAAAAAAGACACAGCGA | 526 |
| | GGGAGAGAGGGAAGGAGGGA | 527 |
| | AGGGAGAGAGGGAAGGAGGG | 528 |
| | GGGAGGGAGAGAGGGAAGGA | 529 |
| | AGGGAGGGAGAGAGGGAAGG | 530 |
| | GAGAGGGAGGGAGAGAGGGA | 531 |
| | AAGGCGAGAGGGAGGGAGAG | 532 |
| | AGGCGAGAGGGAGGGAGAGA | 533 |
| E51, C52, S59, | ACCCACCAATCACGAACCTA | 534 |
| P61 | AAAGGCTCAGCAGTTGACCT | 535 |
| | CAAAGGCTCAGCAGTTGACC | 536 |
| | GAACCTAAGGATGAAAGCAA | 537 |
| I27, L30, M32, | GTCTTTAACACACTCGATAT | 538 |
| W41 | TATCGGTCACATTTCTGTTA | 539 |
| I27, L30, M32 | CACATTTCTGTTAAGGTCCC | 540 |
| I27, L30 | GAGCCTTTGCTTTCATCCTTAGG | 541 |

TABLE 3A
Epitope Residues in CD117

| Antibody | Target Residues in Epitope |
|---|---|
| Ab85 | T67, K69, T71, S81, Y83, T114, T119, K129 |
| Ab65 | S236, H238, Y244, S273, T277, T279 |

TABLE 3B
CD117 Protein Sequence (SEQ ID NO: 3)

QPSVSPGEPSPPSIHPGKSDLIVRVGDEIRLLCTDPGFVKWTFEILDETN
ENKQNEWITEKAEATNTGKYTCTNKHGLSNSIYVFVRDPAKLFLVDRSLY
GKEDNDTLVRCPLTDPEVTNYSLKGCQGKPLPKDLRFIPDPKAGIMIKSV
KRAYHRLCLHCSVDQEGKSVLSEKFILKVRPAFKAVPVVSVSKASYLLRE
GEEFTVTCTIKDVSSSVYSTWKRENSQTKLQEKYNSWHHGDFNYERQATL
TISSARVNDSGVFMCYANNTFGSANVTTTLEVVDKGFINIFPMINTTVFV
NDGENVDLIVEYEAFPKPEHQQWIYMNRTFTDKWEDYPKSENESNIRYVS
ELHLTRLKGTEGGTYTFLVSNSDVNAAIAFNVYVNTKPEILTYDRLVNGM
LQCVAAGFPEPTIDWYFCPGTEQRCSASVLPVDVQTLNSSGPPFGKLVVQ
SSIDSSAFKHNGTVECKAYNDVGKTSAYFNFAFKGNNKEQIHPHTLFTPL
LIGFVIVAGMMCIIVMILTYKYLQKPMYEVQWKVVEEINGNNYVYIDPTQ
LPYDHKWEFPRNRLSFGKTLGAGAFGKVVEATAYGLIKSDAAMTVAVKML
KPSAHLTEREALMSELKVLSYLGNHMNIVNLLGACTIGGPTLVITEYCCY
GDLLNFLRRKRDSFICSKQEDHAEAALYKNLLHSKESSCSDSTNEYMDMK
PGVSYVVPTKADKRRSVRIGSYIERDVTPAIMEDDELALDLEDLLSFSYQ
VAKGMAFLASKNCIHRDLAARNILLTHGRITKICDFGLARDIKNDSNYVV
KGNARLPVKWMAPESIFNCVYTFESDVWSYGIFLWELFSLGSSPYPGMPV
DSKFYKMIKEGFRMLSPEHAPAEMYDIMKTCWDADPLKRPTFKQIVQLIE
KQISESTNHIYSNLANCSPNRQKPVVDHSVRINSVGSTASSSQPLLVHDD
V

TABLE 3C
VH/VL of Ab85

| Region | Sequence | SEQ ID NO: |
|---|---|---|
| VH | EVQLVQSGAEVKKPGESLKISCKGSGYS | 13 |
| | FTNYWIGWVRQMPGKGLEWMAIINPRDS | |
| | DTRYRPSFQGQVTISADKSISTAYLQWS | |
| | SLKASDTAMYYCARHGRGYEGYEGAFDI | |
| | WGQGTLVTVSS | |
| VL | DIQMTQSPSSLSASVGDRVTITCRSSQG | 14 |
| | IRSDLGWYQQKPGKAPKLLIYDASNLET | |
| | GVPSRFSGS GSGTDFTLTISSLQPEDF | |
| | ATYYCQQANGFPLTFGGGTKVEIK | |

TABLE 3D
VH/VL of Ab67

| Region | Sequence | SEQ ID NO: |
|---|---|---|
| VH | EVQLVESGGGLVQPGGSLRLSCAASGFT | 15 |
| | FSDADMDWVRQAPGKGLEWVGRTRNKAG | |
| | SYTTEYAASVKGRFTISRDDSKNSLYLQ | |
| | MNSLKTEDTAVYYCAREPKYWIDFDLWG | |
| | RGTLVTVSS | |
| VL | DIQMTQSPSSLSASVGDRVTITCRASQS | 16 |
| | ISSYLNWYQQKPGKAPKLLIYAASSLQS | |
| | GVPSRFSGSGSGTDFTLTISSLQPEDFA | |
| | TYYCQQSYIAPYTFGGGTKVEIK | |

TABLE 3E
Example spacer sequences of
gRNA for editing CD117 epitope

| | | | | SEQ |
|---|---|---|---|---|
| Res- | Ed- | | | ID |
| idue | itor | Cas | Spacer | NO: |
| T67 | ABE/ | Sp cas9 | aacaccggcaaatacacgtg | 542 |
| | CBE | | ccaacaccggcaaatacacg | 543 |
| | | | caccaacaccggcaaataca | 544 |
| | | | gccaccaacaccggcaaata | 545 |
| | | | aagccaccaacaccggcaaa | 546 |
| | | At cas9 | aacaccggcaaatacacgtgca | 547 |
| | | | caccaacaccggcaaatacacg | 548 |
| | | | accaacaccggcaaatacacgt | 549 |
| | | | gccaccaacaccggcaaataca | 550 |
| | | | gcagaagccaccaacaccggca | 551 |
| | | | ccaacaccggcaaatacacgtg | 552 |
| | | | caacaccggcaaatacacgtgc | 553 |
| | | | caccggcaaatacacgtgcacc | 554 |
| K69 | ABE | Sp cas9 | ccggcaaatacacgtgcacc | 555 |
| | | | accggcaaatacacgtgcac | 556 |
| | | | ggcaaatacacgtgcaccaa | 557 |
| | | | gcaaatacacgtgcaccaac | 558 |
| | | | caaatacacgtgcaccaaca | 559 |
| | | At cas9 | ccggcaaatacacgtgcaccaa | 560 |
| | | | ggcaaatacacgtgcaccaaca | 561 |
| T71 | ABE/ | Sp cas9 | tacacgtgcaccaacaaaca | 562 |
| | CBE | | aatacacgtgcaccaacaaa | 563 |
| | | | acacgtgcaccaacaaacac | 564 |
| | | At cas9 | aaatacacgtgcaccaacaaac | 565 |
| | | | acacgtgcaccaacaaacacgg | 566 |
| S81 | CBE | Sp cas9 | attccatttatgtgtttgtt | 567 |
| | | | aattccatttatgtgtttgt | 568 |
| | | | agcaattccatttatgtgtt | 569 |
| | | | cttaagcaattccatttatg | 570 |
| | | | ggcttaagcaattccattta | 571 |
| | ABE | | ataaatggaattgcttaagc | 572 |
| | | | aaatggaattgcttaagccg | 573 |
| | | | ggaattgcttaagccgtgtt | 574 |
| | | | acacataaatggaattgctt | 575 |
| | | | aacacataaatggaattgct | 576 |
| | | | cacataaatggaattgctta | 577 |
| | ABE | At cas9 | caaacacataaatggaattgct | 578 |
| | | | aaacacataaatggaattgctt | 579 |
| | | | caaacacataaatggaattgct | 580 |
| | CBE | | caattccatttatgtgtttgtt | 581 |
| | | | gcaattccatttatgtgtttgt | 582 |
| | | | gcttaagcaattccatttatgt | 583 |
| Y83 | ABE | Sp cas9 | ttatgtgtttgttagaggta | 584 |
| | | | tttatgtgtttgttagaggt | 585 |
| | | | atttatgtgtttgttagagg | 586 |
| | | | ccatttatgtgtttgttaga | 587 |
| | | | tccatttatgtgtttgttag | 588 |
| | | | ttccatttatgtgtttgtta | 589 |
| | | | tctaacaaacacataaatgg | 590 |
| | | | ctctaacaaacacataaatg | 591 |
| | | | acctctaacaaacacataaa | 592 |
| | | At cas9 | ctctaacaaacacataaatgga | 593 |
| | | | atttatgtgtttgttagaggta | 594 |
| | | | gcaattccatttatgtgtttgt | 595 |
| T114 | ABE/ | Sp cas9 | ctcacagacccagaagtgac | 596 |
| | CBE | | cctctcacagacccagaagt | 597 |
| | | | tcacagacccagaagtgacc | 598 |
| | | | tcctctcacagacccagaag | 599 |
| | | | tgtcctctcacagacccaga | 600 |
| | | | ctgtcctctcacagacccag | 601 |
| | | | gctgtcctctcacagaccca | 602 |
| | | | cgctgtcctctcacagaccc | 603 |
| | | | ccgctgtcctctcacagacc | 604 |
| | | At cas9 | ctgtcctctcacagacccagaa | 605 |
| | | | cacagacccagaagtgaccaat | 606 |
| | | | tgtcctctcacagacccagaag | 607 |
| | | | tcctctcacagacccagaagtg | 608 |
| | | | gctgtcctctcacagacccaga | 609 |
| | | | tccgctgtcctctcacagaccc | 610 |
| T119 | ABE/ | Sp cas9 | gtgaccaattattccctca | 611 |
| | CBE | | gtgaccaattattccctcaa | 612 |
| | | | aagtgaccaattattccctc | 613 |
| | | | gtgaccaattattccctcaa | 614 |
| | | | tgaccaattattccctcaag | 615 |
| | | | gaccaattattccctcaagg | 616 |
| | | | gaagtgaccaattattccct | 617 |
| | | At cas9 | cagacccagaagtgaccaatta | 618 |
| | | | gacccagaagtgaccaattatt | 619 |
| | | | acagacccagaagtgaccaatt | 620 |
| | | | gtgaccaattattccctcaagg | 621 |
| | | | tgaccaattattccctcaaggg | 622 |
| | | | agtgaccaattattccctcaag | 623 |
| K129 | ABE | Sp cas9 | ccaggggaagcctcttccca | 624 |
| | | | caggggaagcctcttcccaa | 625 |
| | | | aggggaagcctcttcccaag | 626 |
| | | | gaagcctcttcccaaggact | 627 |
| | CBE | | ccttgggaagaggcttcccc | 628 |
| | | | tgggaagaggcttcccctgg | 629 |
| | | | aggcttcccctggcacccct | 630 |
| | | | ggcttcccctggcacccctt | 631 |
| | ABE | At cas9 | tgccaggggaagcctcttccca | 632 |
| | | | ggggtgccaggggaagcctctt | 633 |
| | CBE | | ttgggaagaggcttcccctggc | 634 |
| | | | cttgggaagaggcttcccctgg | 635 |
| | | | ccttgggaagaggcttcccctg | 636 |
| | | | agtccttgggaagaggcttccc | 637 |
| | | | aggcttcccctggcaccccttg | 638 |
| | | | ggcttcccctggcaccccttga | 639 |
| | | | aagaggcttcccctggcacccc | 640 |
| | | | agaggcttcccctggcacccct | 641 |
| S236 | ABE | Sp cas9 | aaatataatagctggcatca | 642 |
| | | | tataatagctggcatcacgg | 643 |
| | | | aatataatagctggcatcac | 644 |
| | | | ataatagctggcatcacggt | 645 |
| | | | agaaatataatagctggcat | 646 |
| | | | aggagaaatataatagctgg | 647 |
| | CBE | | gccagctattatatttctcc | 648 |
| | | | caccgtgatgccagctatta | 649 |
| | | | cagctattatatttctcctg | 650 |
| | | | agctattatatttctcctgt | 651 |
| | ABE | At cas9 | tataatagctggcatcacggtg | 652 |
| | | | aaatataatagctggcatcacg | 653 |
| | | | caggagaaatataatagctggc | 654 |
| | | | tacaggagaaatataatagctg | 655 |
| | | | taatagctggcatcacggtgac | 656 |
| | | | tagctggcatcacggtgacttc | 657 |
| | | | ggagaaatataatagctggcat | 658 |
| | CBE | | ccgtgatgccagctattatatt | 659 |
| | | | accgtgatgccagctattatat | 660 |
| | | | tcaccgtgatgccagctattat | 661 |
| H238 | ABE/ | Sp cas9 | agctggcatcacggtgactt | 662 |
| | CBE | | gctggcatcacggtgacttc | 663 |
| | | | ggcatcacggtgacttcaat | 664 |
| | | At cas9 | ggcatcacggtgacttcaatta | 665 |
| | | | gcatcacggtgacttcaattat | 666 |
| Y244 | ABE | Sp cas9 | tgacttcaattatgaacgtc | 667 |
| | | | ttatgaacgtcaggcaacgt | 668 |
| | | | caattatgaacgtcaggcaa | 669 |
| | | | gacttcaattatgaacgtca | 670 |
| | | | acggtgacttcaattatgaa | 671 |
| | | | cataattgaagtcaccgtga | 672 |
| | | | gttcataattgaagtcaccg | 673 |
| | | | acgttcataattgaagtcac | 674 |
| | | | tgcctgacgttcataattga | 675 |
| | | | cgttgcctgacgttcataat | 676 |
| | | | ttcaattatgaacgtcaggc | 677 |
| | | | cttcaattatgaacgtcagg | 678 |
| | | | gtgacttcaattatgaacgt | 679 |
| | | | tcacggtgacttcaattatg | 680 |
| | | | ttcataattgaagtcaccgt | 681 |
| | | | ctgacgttcataattgaagt | 682 |
| | | | ttgcctgacgttcataattg | 683 |
| | | | gttgcctgacgttcataatt | 684 |
| | | At cas9 | caattatgaacgtcaggcaacg | 685 |
| | | | tatgaacgtcaggcaacgttga | 686 |
| | | | tgacttcaattatgaacgtcag | 687 |
| | | | cggtgacttcaattatgaacgt | 688 |
| | | | gttcataattgaagtcaccgtg | 689 |
| | | | cataattgaagtcaccgtgatg | 690 |
| | | | cgttcataattgaagtcaccgt | 691 |
| | | | tgcctgacgttcataattgaag | 692 |
| | | | ttgcctgacgttcataattgaa | 693 |
| | | | cgttgcctgacgttcataattg | 694 |
| | | | tcaattatgaacgtcaggcaac | 695 |
| | | | attatgaacgtcaggcaacgtt | 696 |
| | | | acggtgacttcaattatgaacg | 697 |
| | | | cacggtgacttcaattatgaac | 698 |
| | | | tcataattgaagtcaccgtgat | 699 |
| | | | acgttcataattgaagtcaccg | 700 |
| | | | gcctgacgttcataattgaagt | 701 |
| S273 | CBE | Sp cas9 | ggatcagcaaatgtcacaac | 702 |
| | | | tggatcagcaaatgtcacaa | 703 |
| | | | tttggatcagcaaatgtcac | 704 |
| | | | ttttggatcagcaaatgtca | 705 |
| | | | acttttggatcagcaaatgt | 706 |
| | | | aatacttttggatcagcaaa | 707 |
| | | | ataatacttttggatcagca | 708 |
| | | | aataatacttttggatcagc | 709 |
| | | | caataatacttttggatcag | 710 |
| | ABE | | gctgatccaaaagtattat | 711 |
| | | | tgatccaaaagtattattgg | 712 |
| | | | gctgatccaaaagtattatt | 713 |
| | | | atttgctgatccaaaagtat | 714 |
| | | | gacatttgctgatccaaaag | 715 |
| | | | gtgacatttgctgatccaaa | 716 |
| | | | tgtgacatttgctgatccaa | 717 |
| | | | ttgtgacatttgctgatcca | 718 |
| | | | gttgtgacatttgctgatcc | 719 |
| | | | tgttgtgacatttgctgatc | 720 |
| | ABE | At cas9 | atttgctgatccaaaagtatta | 721 |
| | | | ctgatccaaaagtattattggc | 722 |
| | | | aataatacttttggatcagcaa | 723 |
| | | | ttttggatcagcaaatgtcaca | 724 |
| | | | tttggatcagcaaatgtcacaa | 725 |
| | | | tacttttggatcagcaaatgtc | 726 |
| | | | taatacttttggatcagcaaat | 727 |
| | | | ctgatccaaaagtattattggc | 728 |
| | | | gatccaaaagtattattggcat | 729 |
| | | | tcagcaaatgtcacaacaacct | 730 |
| | | | atacttttggatcagcaaatgt | 731 |
| | | | aatacttttggatcagcaaatg | 732 |
| | | | ataatacttttggatcagcaaa | 733 |
| | | | tttgctgatccaaaagtattat | 734 |
| | | | tgctgatccaaaagtattattg | 735 |
| | | | gttgtgacatttgctgatccaa | 736 |
| | | | acatttgctgatccaaaagtat | 737 |
| | | | ggatcagcaaatgtcacaacaa | 738 |
| T277 | ABE/ | Sp cas9 | agcaaatgtcacaacaacct | 739 |
| | CBE | | gtcacaacaaccttggaagt | 740 |
| | | | cacaacaaccttggaagtag | 741 |
| | | | tgtcacaacaaccttggaag | 742 |
| | | | aatgtcacaacaaccttgga | 743 |
| | | | aaatgtcacaacaaccttgg | 744 |
| | | | caaatgtcacaacaaccttg | 745 |
| | | | gcaaatgtcacaacaacctt | 746 |
| | | | ggatcagcaaatgtcacaac | 747 |
| | | At cas9 | cacaacaaccttggaagtagta | 748 |
| | | | gcaaatgtcacaacaaccttgg | 749 |
| | | | tgtcacaacaaccttggaagta | 750 |
| | | | atgtcacaacaaccttggaagt | 751 |
| T279 | ABE/ | Sp cas9 | caacaaccttggaagtagt | 752 |
| | CBE | | caacaaccttggaagtagta | 753 |
| | | | acaaccttggaagtagtagg | 754 |
| | | | caaccttggaagtagtaggt | 755 |
| | | | aaccttggaagtagtaggta | 756 |
| | | At cas9 | caaccttggaagtagtaggtaa | 757 |
| | | | acaaccttggaagtagtaggta | 758 |

TABLE 3F
Example spacer sequences of
pegRNA for editing CD117 epitope

| Residue | Spacer | SEQ ID NO: |
|---|---|---|
| T67, K69, | CTGATCCGGGCTTTGTCAAATGG | 759 |
| T71, S81, Y83 | TACACGTGCACCAACAAACACGG | 760 |
| | CAAATGGACTTTTGAGATCCTGG | 761 |
| | GAATGAATGGATCACGGAAAAGG | 762 |
| | AAGGCAGAAGCCACCAACACCGG | 763 |
| | ATGAGAATAAGCAGAATGAATGG | 764 |
| | TAAGCAGAATGAATGGATCACGG | 765 |
| | ATTGCTTAAGCCGTGTTTGTTGG | 766 |
| | TGTCATCCAAAATTAAGAGCAGG | 767 |
| | GTTGGTGCACGTGTATTTGCCGG | 768 |
| | ACCTCTAACAAACACATAAATGG | 769 |
| T114, T119, | TTGTTGACCGCTCCTTGTATGGG | 770 |
| K129 | CTTGTTGACCGCTCCTTGTATGG | 771 |
| | GAAAGAAGACAACGACACGCTGG | 772 |
| | TTATTCCCTCAAGGGGTGCCAGG | 773 |
| | TGACCAATTATTCCCTCAAGGGG | 774 |
| | TATTCCCTCAAGGGGTGCCAGGG | 775 |
| | GTGACCAATTATTCCCTCAAGGG | 776 |
| | ATTCCCTCAAGGGGTGCCAGGGG | 777 |
| | TTGATCATGATGCCCGCCTTGGG | 778 |
| | ATGCAGACAGAGCCGATGGTAGG | 779 |
| | TGATCATGATGCCCGCCTTGGGG | 780 |
| | TTTGATCATGATGCCCGCCTTGG | 781 |
| | GCACCCCTTGAGGGAATAATTGG | 782 |
| | AACAATGCAGACAGAGCCGATGG | 783 |
| | GGGAATAATTGGTCACTTCTGGG | 784 |
| | AGGGAATAATTGGTCACTTCTGG | 785 |
| | AGGAATAAACCTCAAGTCCTTGG | 786 |
| | ATGATGCCCGCCTTGGGGTCAGG | 787 |
| | CTTCCCCTGGCACCCCTTGAGGG | 788 |
| | GCTTCCCCTGGCACCCCTTGAGG | 789 |
| | AACCTCAAGTCCTTGGGAAGAGG | 790 |
| | GGAATAAACCTCAAGTCCTTGGG | 791 |
| S236, H238, | AAACCAGCAGACTAAACTACAGG | 792 |
| Y244 | TACAGGAGAAATATAATAGCTGG | 793 |
| | AAATATAATAGCTGGCATCACGG | 794 |
| | GATTCTGAATATAAATTATATGG | 795 |
| | TGCTGATCCAAAAGTATTATTGG | 796 |
| S273, T277, | TCAGCGAGAGTTAATGATTCTGG | 797 |
| T279 | TGACTTCAATTATGAACGTCAGG | 798 |
| | TGTTATGCCAATAATACTTTTGG | 799 |
| | GTATTTACCTACTACTTCCAAGG | 800 |
| | TAATTTAAACATTCCCATAGAGG | 801 |

TABLE 4A
Epitope Residues in CLL-1

| Antibody | Target Residues in Epitope |
|---|---|
| Hu6E7.N54A | 142 to 158 (DSCYFLSDDVQTWQESK) |
| | of SEQ ID NO: 4 |

TABLE 4B
CLL-1 Protein Sequence (SEQ ID NO: 4)

MSEEVTYADLQFQNSSEMEKIPEIGKFGEKAPPAPSHVWRPAALFLTLLC
LLLLIGLGVLASMFHVTLKIEMKKMNKLQNISEELQRNISLQLMSNMNIS
NKIRNLSTTLQTIATKLCRELYSKEQEHKCKPCPRRWIWHKDSCYFLSDD
VQTWQESKMACAAQNASLLKINNKNALEFIKSQSRSYDYWLGLSPEEDST
RGMRVDNIINSSAWVIRNAPDLNNMYCGYINRLYVQYYHCTYKKRMICEK
MANPVQLGSTYFREA

TABLE 4C
VH/VL of Hu6E7.N54A

| Region | Sequence | SEQ ID NO: |
|---|---|---|
| VH | EVQLVQSGAEVKKPGASVKVSCKASGY | 17 |
| | SFTDYYMHWVRQAPGQGLEWIGRINPY | |
| | AGAAFYSQNFKDRVTLTVDTSTSTAYL | |
| | ELSSLRSEDTAVYYCAIERGADLEGYA | |
| | MDYWGQGTLVTVSS | |
| VL | DIQMTQSPSSLSASVGDRVTITCRASQ | 18 |
| | SVSTSSYNYMHWYQQKPGKPPKLLIKY | |
| | ASNLESGVPSRFSGSGSGTDFTLTISS | |
| | LQPEDFATYYCQHSWEIPLTFGQGTKV | |
| | EIK | |

TABLE 4D
Example spacer sequences of
gRNA for editing CLL-1 epitope

| | | | | SEQ |
|---|---|---|---|---|
| Res- | Ed- | | | ID |
| idues | itor | Cas | Spacer | NO: |
| 142- | ABE | Sp cas9 | taagtgatgatgtccaaaca | 802 |
| 158 | | | tgatgatgtccaaacatggc | 803 |
| | | | aacatggcaggagagtaaaa | 804 |
| | ABE/CBE | | atgtttggacatcatcactt | 805 |
| | CBE | | ttttactctcctgccatgtt | 806 |
| | ABE | | aggacagctgttatttccta | 807 |
| | | | gacagctgttatttcctaag | 808 |
| | | | agctgttatttcctaagtga | 809 |
| | ABE/CBE | | tgttatttcctaagtgatga | 810 |
| | | | tgatgtccaaacatggcagg | 811 |
| | | | atgtccaaacatggcaggag | 812 |
| | | | ggcaggagagtaaaatggcc | 813 |
| | | | caggagagtaaaatggcctg | 814 |
| | | | ggaaataacagctgtcctta | 815 |
| | | | atcacttaggaaataacagc | 816 |
| | | | atcatcacttaggaaataac | 817 |
| | | | gccattttactctcctgcca | 818 |
| | ABE | | taaggacagctgttatttcc | 819 |
| | | | aaggacagctgttatttcct | 820 |
| | | | acagctgttatttcctaagt | 821 |
| | | | gctgttatttcctaagtgat | 822 |
| | | | atttcctaagtgatgatgtc | 823 |
| | | | ttcctaagtgatgatgtcca | 824 |
| | | | cctaagtgatgatgtccaaa | 825 |
| | | | atgatgtccaaacatggcag | 826 |
| | ABE/CBE | | gatgtccaaacatggcagga | 827 |
| | | | gtccaaacatggcaggagag | 828 |
| | | | tccaaacatggcaggagagt | 829 |
| | | | caaacatggcaggagagtaa | 830 |
| | CBE | | gctgtccttatgccaaatc | 831 |
| | ABE/CBE | | taacagctgtccttatgcca | 832 |
| | | | ataacagctgtccttatgcc | 833 |
| | | | aataacagctgtccttatgc | 834 |
| | | | catcatcacttaggaaataa | 835 |
| | | | gacatcatcacttaggaaat | 836 |
| | | | ttggacatcatcacttagga | 837 |
| | | | tttggacatcatcacttagg | 838 |
| | | | gtttggacatcatcacttag | 839 |
| | | | ctgccatgtttggacatcat | 840 |
| | | | ctcctgccatgtttggacat | 841 |
| | | | actctcctgccatgtttgga | 842 |
| | | | ttactctcctgccatgtttg | 843 |
| | | | aggccattttactctcctgc | 844 |
| | ABE | At cas9 | agctgttatttcctaagtgatg | 845 |
| | | | agctgttatttcctaagtgatg | 846 |
| | | | tttggcataaggacagctgtta | 847 |
| | ABE/CBE | | ttaggaaataacagctgtcctt | 848 |
| | | | cttaggaaataacagctgtcct | 849 |
| | | | tcatcacttaggaaataacagc | 850 |
| | | | tctcctgccatgtttggacatc | 851 |
| | ABE | | taaggacagctgttatttccta | 852 |
| | ABE/CBE | | tgttatttcctaagtgatgatg | 853 |
| | ABE | | cctaagtgatgatgtccaaaca | 854 |
| | ABE/CBE | | gtgatgatgtccaaacatggca | 855 |
| | | | atgatgtccaaacatggcagga | 856 |
| | | | tgtttggacatcatcacttagg | 857 |
| | | | cctgccatgtttggacatcatc | 858 |
| | | | ttactctcctgccatgtttgga | 859 |
| | | | attttactctcctgccatgttt | 860 |
| | | | ttatttcctaagtgatgatgtc | 861 |
| | ABE | | tcctaagtgatgatgtccaaac | 862 |
| | ABE/CBE | | ccaaacatggcaggagagtaaa | 863 |
| | | | caaacatggcaggagagtaaaa | 864 |
| | | | atggcaggagagtaaaatggcc | 865 |
| | | | gcaggagagtaaaatggcctgt | 866 |
| | | | ggacatcatcacttaggaaata | 867 |
| | | | ttggacatcatcacttaggaaa | 868 |
| | | | actctcctgccatgtttggaca | 869 |
| | | | tttactctcctgccatgtttgg | 870 |
| | CBE | | cattttactctcctgccatgtt | 871 |
| | ABE | | gcataaggacagctgttatttc | 872 |
| | | | tttcctaagtgatgatgtccaa | 873 |
| | ABE/CBE | | taagtgatgatgtccaaacatg | 874 |
| | | | tgatgatgtccaaacatggcag | 875 |
| | | | acttaggaaataacagctgtcc | 876 |
| | | | tgccatgtttggacatcatcac | 877 |
| | | | ctgccatgtttggacatcatca | 878 |
| | | | gccattttactctcctgccatg | 879 |

TABLE 4F
Example spacer sequences of
pegRNA for editing CLL-1 epitope

| Residues | Spacer | SEQ ID NO: |
|---|---|---|
| 142-158 | TAAGTGATGATGTCCAAACATGG | 880 |
| | TGATGATGTCCAAACATGGCAGG | 881 |
| | ACAAATGTAAGCCTTGTCCAAGG | 882 |
| | CTTGTCCAAGGAGATGGATTTGG | 883 |
| | GTAAGCCTTGTCCAAGGAGATGG | 884 |
| | AAGGAGATGGATTTGGCATAAGG | 885 |
| | CCCATGATGGTAGAAACACCTGG | 886 |
| | CCATGATGGTAGAAACACCTGGG | 887 |
| | CACCCCTCTCTATCCCATGATGG | 888 |
| | GTTGTTTATCTTCAACAGGCTGG | 889 |
| | ATGTTTGGACATCATCACTTAGG | 890 |
| | GCTGGCATTCTGAGCAGCACAGG | 891 |
| | TTTTGTTGTTTATCTTCAACAGG | 892 |
| | TTTTACTCTCCTGCCATGTTTGG | 893 |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A mutant CD123 protein comprising a mutation at residue R84 according to SEQ ID NO:2, wherein the mutation is to an amino acid residue that is not lysine.

2. The mutant CD123 protein of claim 1, wherein the mutation is to glutamine (Q), asparagine (N) or histidine (H).

3. The mutant CD123 protein of claim 1, wherein the mutation is R84Q.

4. The mutant CD123 protein of claim 1, which further comprises a mutation at residue V85 according to SEQ ID NO:2.

5. The mutant CD123 protein of claim 3, wherein the mutation at residue V85 is to methionine (M), isoleucine (I), leucine (L), alanine (A), cysteine (C), glycine (G), or threonine (T).

6. The mutant CD123 protein of claim 4, wherein the mutations are selected from the group consisting of R84Q and V85I , R84Q and V85M, R84H and V85I , and R84H and V85M.

7. The mutant CD123 protein of claim 1, which comprises an amino acid sequence selected from the group consisting of SEQ ID NO: 894, 895, 896 and 897.

8. A polynucleotide encoding the mutant CD123 protein of claim 1.

9. A cell comprising the mutant CD123 protein of claim 1 or a polynucleotide encoding the mutant CD123 protein.

10. A method for preparing a cancer patient for a therapy comprising an anti-CD123 antibody or antigen-binding fragment thereof, comprising administering to the patient a cell expressing the mutant CD123 protein of claim 1 which has reduced binding to the anti-CD123 antibody or antigen-binding fragment thereof as compared to the corresponding wild-type CD123 protein.

11. The method of claim 10, wherein the cell is a stem cell.

12. The method of claim 11, wherein the stem cell is a hematopoietic stem and progenitor cell (HSPC).

13. The method of claim 10, wherein the therapy comprises the antibody, an antigen-binding fragment of the antibody, a chimeric antigen receptor (CAR) comprising the antigen-binding fragment, or an immune cell comprising the CAR.

14. The method of claim 10, wherein the cancer is leukemia.

15. The method of claim 10, wherein the cancer is acute myeloid leukemia (AML).

16. The method of claim 10, wherein the anti-CD123 antibody is CSL362 or 32716.

17. A method for preparing the polynucleotide of claim 8 in a cell, comprising introducing to the cell with a base editor comprising a gRNA that comprises a spacer sequence selected from the group consisting of SEQ ID NO:229-516.

18. A method for preparing the polynucleotide of claim 8 in a cell, comprising introducing to the cell a prime editor and a pegRNA that comprises a spacer sequence selected from the group consisting of SEQ ID NO:517-541.


SEQUENCE LISTING (897 sequences)

SEQ ID NO: 1 - protein, 364 aa, synthetic construct
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 378 aa, synthetic construct
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 3 - protein, 951 aa, synthetic construct
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 4 - protein, 265 aa, synthetic construct
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 5 - protein, 118 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 6 - protein, 113 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 7 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 8 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 9 - protein, 180 aa, synthetic construct
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSS

SEQ ID NO: 10 - protein, 112 aa, synthetic construct
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQ

SEQ ID NO: 11 - protein, 118 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 12 - protein, 111 aa, synthetic construct
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 13 - protein, 123 aa, synthetic construct
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 14 - protein, 107 aa, synthetic construct
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 15 - protein, 121 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 16 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 17 - protein, 122 aa, synthetic construct
EVQLVQSGAEVKKPGASVKVSCKASGYSFTDYYMHWVRQAPGQGLEWIGRINPYAGAAFYSQNFKDRVTLTVDTSTSTAYLELSSLRSEDTAVYYCAIERGADLEGYAMDYWGQGTLVTVSS

SEQ ID NO: 18 - protein, 111 aa, synthetic construct
DIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIK

SEQ ID NO: 19 - RNA, 20 nt, synthetic construct
  source 1..20: mol_type = other RNA
gtgcagggcacgaggacgca

SEQ ID NO: 20 - RNA, 20 nt, synthetic construct
  source 1..20: mol_type = other RNA
aagtgcagggcacgaggacg

SEQ ID NO: 21 - RNA, 20 nt, synthetic construct
  source 1..20: mol_type = other RNA
gaaagtgcagggcacgagga

SEQ ID NO: 22 - RNA, 20 nt, synthetic construct
  source 1..20: mol_type = other RNA
aagaaagtgcagggcacgag

SEQ ID NO: 23 - RNA, 20 nt, synthetic construct
  source 1..20: mol_type = other RNA
gaagaaagtgcagggcacga

SEQ ID NO: 24 - RNA, 20 nt, synthetic construct
  source 1..20: mol_type = other RNA
ggaagaaagtgcagggcacg

SEQ ID NO: 25 - RNA, 20 nt, synthetic construct
  source 1..20: mol_type = other RNA
tggaagaaagtgcagggcac

SEQ ID NO: 26 - RNA, 20 nt, synthetic construct
  source 1..20: mol_type = other RNA
tgcagggcacgaggacgcac

SEQ ID NO: 27 - RNA, 20 nt, synthetic construct
  source 1..20: mol_type = other RNA
atggaagaaagtgcagggca

SEQ ID NO: 28 - RNA, 20 nt, synthetic construct
  source 1..20: mol_type = other RNA
ggatggaagaaagtgcaggg

SEQ ID NO: 29 - RNA, 20 nt, synthetic construct
  source 1..20: mol_type = other RNA
tgggatggaagaaagtgcag

SEQ ID NO: 30 - RNA, 22 nt, synthetic construct
  source 1..22: mol_type = other RNA
atggaagaaagtgcagggcacg

SEQ ID NO: 31 - RNA, 22 nt, synthetic construct
  source 1..22: mol_type = other RNA
ggaagaaagtgcagggcacgag

SEQ ID NO: 32 - RNA, 22 nt, synthetic construct
  source 1..22: mol_type = other RNA
aagaaagtgcagggcacgagga

SEQ ID NO: 33 - RNA, 22 nt, synthetic construct
  source 1..22: mol_type = other RNA
gaagaaagtgcagggcacgagg

SEQ ID NO: 34 - RNA, 20 nt, synthetic construct
  source 1..20: mol_type = other RNA
cggaaccagtaaccatgaac

SEQ ID NO: 35 - RNA, 20 nt, synthetic construct
  source 1..20: mol_type = other RNA
ggaaccagtaaccatgaact

SEQ ID NO: 36 - RNA, 20 nt, synthetic construct
  source 1..20: mol_type = other RNA
gaaccagtaaccatgaactg

SEQ ID NO: 37 - RNA, 20 nt, synthetic construct
  source 1..20: mol_type = other RNA
accagtaaccatgaactggg

SEQ ID NO: 38 - RNA, 20 nt, synthetic construct
  source 1..20: mol_type = other RNA
aaccagtaaccatgaactgg

SEQ ID NO: 39 - RNA, 20 nt, synthetic construct
  source 1..20: mol_type = other RNA
ccagtaaccatgaactgggg

SEQ ID NO: 40 - RNA, 20 nt, synthetic construct
  source 1..20: mol_type = other RNA
cttcccggaaccagtaacca

SEQ ID NO: 41 - RNA, 20 nt, synthetic construct
  source 1..20: mol_type = other RNA
tcccggaaccagtaaccatg

SEQ ID NO: 42 - RNA, 20 nt, synthetic construct
  source 1..20: mol_type = other RNA
ttcccggaaccagtaaccat

SEQ ID NO: 43 - RNA, 20 nt, synthetic construct
  source 1..20: mol_type = other RNA
tccttcccggaaccagtaac

SEQ ID NO: 44 - RNA, 20 nt, synthetic construct
  source 1..20: mol_type = other RNA
ggctccttcccggaaccagt

SEQ ID NO: 45 - RNA, 22 nt, synthetic construct
  source 1..22: mol_type = other RNA
aaccagtaaccatgaactgggg

SEQ ID NO: 46 - RNA, 22 nt, synthetic construct
  source 1..22: mol_type = other RNA
ttcccggaaccagtaaccatga

SEQ ID NO: 47 - RNA, 20 nt, synthetic construct
  source 1..20: mol_type = other RNA
tacgatgctcagggagcagt

SEQ ID NO: 48 - RNA, 20 nt, synthetic construct
  source 1..20: mol_type = other RNA
gtctacgatgctcagggagc

SEQ ID NO: 49 - RNA, 20 nt, synthetic construct
  source 1..20: mol_type = other RNA
cgtctacgatgctcagggag

SEQ ID NO: 50 - RNA, 20 nt, synthetic construct
  source 1..20: mol_type = other RNA
ggcgtctacgatgctcaggg

SEQ ID NO: 51 - RNA, 20 nt, synthetic construct
  source 1..20: mol_type = other RNA
tggcgtctacgatgctcagg

SEQ ID NO: 52 - RNA, 20 nt, synthetic construct
  source 1..20: mol_type = other RNA
ctggcgtctacgatgctcag

SEQ ID NO: 53 - RNA, 20 nt, synthetic construct
  source 1..20: mol_type = other RNA
cctggcgtctacgatgctca

SEQ ID NO: 54 - RNA, 20 nt, synthetic construct
  source 1..20: mol_type = other RNA
tcctggcgtctacgatgctc

SEQ ID NO: 55 - RNA, 20 nt, synthetic construct
  source 1..20: mol_type = other RNA
ctcctggcgtctacgatgct

SEQ ID NO: 56 - RNA, 22 nt, synthetic construct
  source 1..22: mol_type = other RNA
tctacgatgctcagggagcagt

SEQ ID NO: 57 - RNA, 22 nt, synthetic construct
  source 1..22: mol_type = other RNA
ctacgatgctcagggagcagtt

SEQ ID NO: 58 - RNA, 22 nt, synthetic construct
  source 1..22: mol_type = other RNA
cgatgctcagggagcagttgtt

SEQ ID NO: 59 - RNA, 22 nt, synthetic construct
  source 1..22: mol_type = other RNA
ggagggataatggttcatactt

SEQ ID NO: 60 - RNA, 22 nt, synthetic construct
  source 1..22: mol_type = other RNA
ggaggagggataatggttcata

SEQ ID NO: 61 - RNA, 22 nt, synthetic construct
  source 1..22: mol_type = other RNA
aggaggagggataatggttcat

SEQ ID NO: 62 - RNA, 20 nt, synthetic construct
  source 1..20: mol_type = other RNA
ataatggttcatacttcttt

SEQ ID NO: 63 - RNA, 20 nt, synthetic construct
  source 1..20: mol_type = other RNA
gacgccaggaggagggataa

SEQ ID NO: 64 - RNA, 20 nt, synthetic construct
  source 1..20: mol_type = other RNA
ggaggagggataatggttca

SEQ ID NO: 65 - RNA, 20 nt, synthetic construct
  source 1..20: mol_type = other RNA
caggaggagggataatggtt

SEQ ID NO: 66 - RNA, 20 nt, synthetic construct
  source 1..20: mol_type = other RNA
ttcatacttctttcggatgg

SEQ ID NO: 67 - RNA, 20 nt, synthetic construct
  source 1..20: mol_type = other RNA
tcatacttctttcggatgga

SEQ ID NO: 68 - RNA, 20 nt, synthetic construct
  source 1..20: mol_type = other RNA
gttcatacttctttcggatg

SEQ ID NO: 69 - RNA, 20 nt, synthetic construct
  source 1..20: mol_type = other RNA
ccgaaagaagtatgaaccat

SEQ ID NO: 70 - RNA, 22 nt, synthetic construct
  source 1..22: mol_type = other RNA
aatggttcatacttctttcgga

SEQ ID NO: 71 - RNA, 22 nt, synthetic construct
  source 1..22: mol_type = other RNA
ggttcatacttctttcggatgg

SEQ ID NO: 72 - RNA, 22 nt, synthetic construct
  source 1..22: mol_type = other RNA
catacttctttcggatggagag

SEQ ID NO: 73 - RNA, 20 nt, synthetic construct
  source 1..20: mol_type = other RNA
ccgaaagaagtatgaaccat

SEQ ID NO: 74 - RNA, 20 nt, synthetic construct
  source 1..20: mol_type = other RNA
catccgaaagaagtatgaac

SEQ ID NO: 75 - RNA, 20 nt, synthetic construct
  source 1..20: mol_type = other RNA
ctctctccatccgaaagaag

SEQ ID NO: 76 - RNA, 22 nt, synthetic construct
  source 1..22: mol_type = other RNA
catccgaaagaagtatgaacca

SEQ ID NO: 77 - RNA, 22 nt, synthetic construct
  source 1..22: mol_type = other RNA
atccgaaagaagtatgaaccat

SEQ ID NO: 78 - RNA, 22 nt, synthetic construct
  source 1..22: mol_type = other RNA
tccgaaagaagtatgaaccatt

SEQ ID NO: 79 - RNA, 22 nt, synthetic construct
  source 1..22: mol_type = other RNA
cgaaagaagtatgaaccattat

SEQ ID NO: 80 - RNA, 22 nt, synthetic construct
  source 1..22: mol_type = other RNA
ctctccatccgaaagaagtatg

SEQ ID NO: 81 - RNA, 20 nt, synthetic construct
  source 1..20: mol_type = other RNA
caaatctccccagctctctg

SEQ ID NO: 82 - RNA, 20 nt, synthetic construct
  source 1..20: mol_type = other RNA
tctccccagctctctgtgca

SEQ ID NO: 83 - RNA, 20 nt, synthetic construct
  source 1..20: mol_type = other RNA
aatctccccagctctctgtg

SEQ ID NO: 84 - RNA, 20 nt, synthetic construct
  source 1..20: mol_type = other RNA
tacaaatctccccagctctc

SEQ ID NO: 85 - RNA, 22 nt, synthetic construct
  source 1..22: mol_type = other RNA
atctccccagctctctgtgcat

SEQ ID NO: 86 - RNA, 22 nt, synthetic construct
  source 1..22: mol_type = other RNA
gttacaaatctccccagctctc

SEQ ID NO: 87 - RNA, 22 nt, synthetic construct
  source 1..22: mol_type = other RNA
atacagttacaaatctccccag

SEQ ID NO: 88 - RNA, 22 nt, synthetic construct
  source 1..22: mol_type = other RNA
aaatctccccagctctctgtgc

SEQ ID NO: 89 - RNA, 22 nt, synthetic construct
  source 1..22: mol_type = other RNA
acaaatctccccagctctctgt

SEQ ID NO: 90 - RNA, 20 nt, synthetic construct
  source 1..20: mol_type = other RNA
agaaatttggatccatagcc

SEQ ID NO: 91 - RNA, 20 nt, synthetic construct
  source 1..20: mol_type = other RNA
tgcacttgcagccagaaatt

SEQ ID NO: 92 - RNA, 20 nt, synthetic construct
  source 1..20: mol_type = other RNA
agccagaaatttggatccat

SEQ ID NO: 93 - RNA, 20 nt, synthetic construct
  source 1..20: mol_type = other RNA
cagccagaaatttggatcca

SEQ ID NO: 94 - RNA, 20 nt, synthetic construct
  source 1..20: mol_type = other RNA
tgcagccagaaatttggatc

SEQ ID NO: 95 - RNA, 20 nt, synthetic construct
  source 1..20: mol_type = other RNA
cacttgcagccagaaatttg

SEQ ID NO: 96 - RNA, 20 nt, synthetic construct
  source 1..20: mol_type = other RNA
gcacttgcagccagaaattt

SEQ ID NO: 97 - RNA, 22 nt, synthetic construct
  source 1..22: mol_type = other RNA
tgcacttgcagccagaaatttg

SEQ ID NO: 98 - RNA, 22 nt, synthetic construct
  source 1..22: mol_type = other RNA
tgcagccagaaatttggatcca

SEQ ID NO: 99 - RNA, 22 nt, synthetic construct
  source 1..22: mol_type = other RNA
ccagaaatttggatccatagcc

SEQ ID NO: 100 - RNA, 22 nt, synthetic construct
  source 1..22: mol_type = other RNA
cttgcagccagaaatttggatc

SEQ ID NO: 101 - RNA, 20 nt, synthetic construct
  source 1..20: mol_type = other RNA
aaaccctcctgtaccgtcac

SEQ ID NO: 102 - RNA, 20 nt, synthetic construct
  source 1..20: mol_type = other RNA
cacaaaccctcctgtaccgt

SEQ ID NO: 103 - RNA, 20 nt, synthetic construct
  source 1..20: mol_type = other RNA
acgcacaaaccctcctgtac

SEQ ID NO: 104 - RNA, 20 nt, synthetic construct
  source 1..20: mol_type = other RNA
aggacgcacaaaccctcctg

SEQ ID NO: 105 - RNA, 20 nt, synthetic construct
  source 1..20: mol_type = other RNA
cgaggacgcacaaaccctcc

SEQ ID NO: 106 - RNA, 22 nt, synthetic construct
  source 1..22: mol_type = other RNA
gacgcacaaaccctcctgtacc

SEQ ID NO: 107 - RNA, 22 nt, synthetic construct
  source 1..22: mol_type = other RNA
aaaccctcctgtaccgtcactg

SEQ ID NO: 108 - RNA, 22 nt, synthetic construct
  source 1..22: mol_type = other RNA
caaaccctcctgtaccgtcact

SEQ ID NO: 109 - RNA, 22 nt, synthetic construct
  source 1..22: mol_type = other RNA
cacaaaccctcctgtaccgtca

SEQ ID NO: 110 - RNA, 22 nt, synthetic construct
  source 1..22: mol_type = other RNA
cgaggacgcacaaaccctcctg

SEQ ID NO: 111 - RNA, 20 nt, synthetic construct
  source 1..20: mol_type = other RNA
aggaggcggaatctgccctg

SEQ ID NO: 112 - RNA, 20 nt, synthetic construct
  source 1..20: mol_type = other RNA
aaggaggcggaatctgccct

SEQ ID NO: 113 - RNA, 22 nt, synthetic construct
  source 1..22: mol_type = other RNA
aggcggaatctgccctgagtct

SEQ ID NO: 114 - RNA, 22 nt, synthetic construct
  source 1..22: mol_type = other RNA
gaatctgccctgagtctcctcc

SEQ ID NO: 115 - RNA, 22 nt, synthetic construct
  source 1..22: mol_type = other RNA
gaggcggaatctgccctgagtc

SEQ ID NO: 116 - RNA, 22 nt, synthetic construct
  source 1..22: mol_type = other RNA
aggaggcggaatctgccctgag

SEQ ID NO: 117 - RNA, 22 nt, synthetic construct
  source 1..22: mol_type = other RNA
aaggaggcggaatctgccctga

SEQ ID NO: 118 - RNA, 22 nt, synthetic construct
  source 1..22: mol_type = other RNA
ccaaggaggcggaatctgccct

SEQ ID NO: 119 - RNA, 20 nt, synthetic construct
  source 1..20: mol_type = other RNA
aacaactgctccctgagcat

SEQ ID NO: 120 - RNA, 20 nt, synthetic construct
  source 1..20: mol_type = other RNA
aggaacaactgctccctgag

SEQ ID NO: 121 - RNA, 20 nt, synthetic construct
  source 1..20: mol_type = other RNA
gtaggaacaactgctccctg

SEQ ID NO: 122 - RNA, 20 nt, synthetic construct
  source 1..20: mol_type = other RNA
agtaggaacaactgctccct

SEQ ID NO: 123 - RNA, 20 nt, synthetic construct
  source 1..20: mol_type = other RNA
cagtaggaacaactgctccc

SEQ ID NO: 124 - RNA, 22 nt, synthetic construct
  source 1..22: mol_type = other RNA
gggatcccagtaggaacaactg

SEQ ID NO: 125 - RNA, 22 nt, synthetic construct
  source 1..22: mol_type = other RNA
agtaggaacaactgctccctga

SEQ ID NO: 126 - RNA, 22 nt, synthetic construct
  source 1..22: mol_type = other RNA
cccagtaggaacaactgctccc

SEQ ID NO: 127 - RNA, 22 nt, synthetic construct
  source 1..22: mol_type = other RNA
ggggatcccagtaggaacaact

SEQ ID NO: 128 - RNA, 22 nt, synthetic construct
  source 1..22: mol_type = other RNA
atcccagtaggaacaactgctc

SEQ ID NO: 129 - RNA, 20 nt, synthetic construct
  source 1..20: mol_type = other RNA
ataatggttcatacttcttt

SEQ ID NO: 130 - RNA, 20 nt, synthetic construct
  source 1..20: mol_type = other RNA
ggaggagggataatggttca

SEQ ID NO: 131 - RNA, 22 nt, synthetic construct
  source 1..22: mol_type = other RNA
ggagggataatggttcatactt

SEQ ID NO: 132 - RNA, 22 nt, synthetic construct
  source 1..22: mol_type = other RNA
aggaggagggataatggttcat

SEQ ID NO: 133 - RNA, 22 nt, synthetic construct
  source 1..22: mol_type = other RNA
ataatggttcatacttctttcg

SEQ ID NO: 134 - RNA, 20 nt, synthetic construct
  source 1..20: mol_type = other RNA
gggagatttgtaactgtatt

SEQ ID NO: 135 - RNA, 20 nt, synthetic construct
  source 1..20: mol_type = other RNA
gagctggggagatttgtaac

SEQ ID NO: 136 - RNA, 20 nt, synthetic construct
  source 1..20: mol_type = other RNA
gctggggagatttgtaactg

SEQ ID NO: 137 - RNA, 20 nt, synthetic construct
  source 1..20: mol_type = other RNA
tctccccagctctctgtgca

SEQ ID NO: 138 - RNA, 20 nt, synthetic construct
  source 1..20: mol_type = other RNA
caaatctccccagctctctg

SEQ ID NO: 139 - RNA, 20 nt, synthetic construct
  source 1..20: mol_type = other RNA
tacaaatctccccagctctc

SEQ ID NO: 140 - RNA, 20 nt, synthetic construct
  source 1..20: mol_type = other RNA
aatctccccagctctctgtg

SEQ ID NO: 141 - RNA, 22 nt, synthetic construct
  source 1..22: mol_type = other RNA
atctccccagctctctgtgcat

SEQ ID NO: 142 - RNA, 22 nt, synthetic construct
  source 1..22: mol_type = other RNA
acaaatctccccagctctctgt

SEQ ID NO: 143 - RNA, 22 nt, synthetic construct
  source 1..22: mol_type = other RNA
aaatctccccagctctctgtgc

SEQ ID NO: 144 - RNA, 22 nt, synthetic construct
  source 1..22: mol_type = other RNA
agctggggagatttgtaactgt

SEQ ID NO: 145 - RNA, 23 nt, synthetic construct
  source 1..23: mol_type = other RNA
tggctatggatccaaatttctgg

SEQ ID NO: 146 - RNA, 23 nt, synthetic construct
  source 1..23: mol_type = other RNA
aaatttctggctgcaagtgcagg

SEQ ID NO: 147 - RNA, 23 nt, synthetic construct
  source 1..23: mol_type = other RNA
gcaggagtcagtgacggtacagg

SEQ ID NO: 148 - RNA, 23 nt, synthetic construct
  source 1..23: mol_type = other RNA
ggagtcagtgacggtacaggagg

SEQ ID NO: 149 - RNA, 23 nt, synthetic construct
  source 1..23: mol_type = other RNA
gagtcagtgacggtacaggaggg

SEQ ID NO: 150 - RNA, 23 nt, synthetic construct
  source 1..23: mol_type = other RNA
gggagaggggttgtcgggctggg

SEQ ID NO: 151 - RNA, 23 nt, synthetic construct
  source 1..23: mol_type = other RNA
gtgggcaggtgagtggctgtggg

SEQ ID NO: 152 - RNA, 23 nt, synthetic construct
  source 1..23: mol_type = other RNA
ggggagaggggttgtcgggctgg

SEQ ID NO: 153 - RNA, 23 nt, synthetic construct
  source 1..23: mol_type = other RNA
tcgtttccccacaggggccctgg

SEQ ID NO: 154 - RNA, 23 nt, synthetic construct
  source 1..23: mol_type = other RNA
ctgtggggagaggggttgtcggg

SEQ ID NO: 155 - RNA, 23 nt, synthetic construct
  source 1..23: mol_type = other RNA
ccccacaggggccctggctatgg

SEQ ID NO: 156 - RNA, 23 nt, synthetic construct
  source 1..23: mol_type = other RNA
gcaagtgcaggagtcagtgacgg

SEQ ID NO: 157 - RNA, 23 nt, synthetic construct
  source 1..23: mol_type = other RNA
gctgaccctcgtttccccacagg

SEQ ID NO: 158 - RNA, 23 nt, synthetic construct
  source 1..23: mol_type = other RNA
tgaccctcgtttccccacagggg

SEQ ID NO: 159 - RNA, 23 nt, synthetic construct
  source 1..23: mol_type = other RNA
ctgaccctcgtttccccacaggg

SEQ ID NO: 160 - RNA, 23 nt, synthetic construct
  source 1..23: mol_type = other RNA
tgtgggcaggtgagtggctgtgg

SEQ ID NO: 161 - RNA, 23 nt, synthetic construct
  source 1..23: mol_type = other RNA
tgggcaggtgagtggctgtgggg

SEQ ID NO: 162 - RNA, 23 nt, synthetic construct
  source 1..23: mol_type = other RNA
gctgtggggagaggggttgtcgg

SEQ ID NO: 163 - RNA, 23 nt, synthetic construct
  source 1..23: mol_type = other RNA
ccctgctgtgggcaggtgagtgg

SEQ ID NO: 164 - RNA, 23 nt, synthetic construct
  source 1..23: mol_type = other RNA
gtgagtggctgtggggagagggg

SEQ ID NO: 165 - RNA, 23 nt, synthetic construct
  source 1..23: mol_type = other RNA
ggtgagtggctgtggggagaggg

SEQ ID NO: 166 - RNA, 23 nt, synthetic construct
  source 1..23: mol_type = other RNA
aggtgagtggctgtggggagagg

SEQ ID NO: 167 - RNA, 23 nt, synthetic construct
  source 1..23: mol_type = other RNA
ggggagttcttgtcgtagtaggg

SEQ ID NO: 168 - RNA, 23 nt, synthetic construct
  source 1..23: mol_type = other RNA
tggggagttcttgtcgtagtagg

SEQ ID NO: 169 - RNA, 23 nt, synthetic construct
  source 1..23: mol_type = other RNA
gttcttgtcgtagtagggtatgg

SEQ ID NO: 170 - RNA, 23 nt, synthetic construct
  source 1..23: mol_type = other RNA
ttcttgtcgtagtagggtatggg

SEQ ID NO: 171 - RNA, 23 nt, synthetic construct
  source 1..23: mol_type = other RNA
tggagagtccctggatataatgg

SEQ ID NO: 172 - RNA, 23 nt, synthetic construct
  source 1..23: mol_type = other RNA
gaaccagtaaccatgaactgggg

SEQ ID NO: 173 - RNA, 23 nt, synthetic construct
  source 1..23: mol_type = other RNA
tgtcgtagtagggtatgggatgg

SEQ ID NO: 174 - RNA, 23 nt, synthetic construct
  source 1..23: mol_type = other RNA
tggatataatggctccttcccgg

SEQ ID NO: 175 - RNA, 23 nt, synthetic construct
  source 1..23: mol_type = other RNA
tgtggccactggagagtccctgg

SEQ ID NO: 176 - RNA, 23 nt, synthetic construct
  source 1..23: mol_type = other RNA
cggaaccagtaaccatgaactgg

SEQ ID NO: 177 - RNA, 23 nt, synthetic construct
  source 1..23: mol_type = other RNA
ggaagaaagtgcagggcacgagg

SEQ ID NO: 178 - RNA, 23 nt, synthetic construct
  source 1..23: mol_type = other RNA
atgggatggaagaaagtgcaggg

SEQ ID NO: 179 - RNA, 23 nt, synthetic construct
  source 1..23: mol_type = other RNA
tatgggatggaagaaagtgcagg

SEQ ID NO: 180 - RNA, 23 nt, synthetic construct
  source 1..23: mol_type = other RNA
ggaaccagtaaccatgaactggg

SEQ ID NO: 181 - RNA, 23 nt, synthetic construct
  source 1..23: mol_type = other RNA
gacaagaactccccagttcatgg

SEQ ID NO: 182 - RNA, 23 nt, synthetic construct
  source 1..23: mol_type = other RNA
tggagagtccctggatataatgg

SEQ ID NO: 183 - RNA, 23 nt, synthetic construct
  source 1..23: mol_type = other RNA
tggatataatggctccttcccgg

SEQ ID NO: 184 - RNA, 23 nt, synthetic construct
  source 1..23: mol_type = other RNA
tctagcttgtttgtggccactgg

SEQ ID NO: 185 - RNA, 23 nt, synthetic construct
  source 1..23: mol_type = other RNA
ttcttgatctagcttgtttgtgg

SEQ ID NO: 186 - RNA, 23 nt, synthetic construct
  source 1..23: mol_type = other RNA
tgtggccactggagagtccctgg

SEQ ID NO: 187 - RNA, 23 nt, synthetic construct
  source 1..23: mol_type = other RNA
gggaaggagccattatatccagg

SEQ ID NO: 188 - RNA, 23 nt, synthetic construct
  source 1..23: mol_type = other RNA
gcctccttggggatcccagtagg

SEQ ID NO: 189 - RNA, 23 nt, synthetic construct
  source 1..23: mol_type = other RNA
ggaaggagccattatatccaggg

SEQ ID NO: 190 - RNA, 23 nt, synthetic construct
  source 1..23: mol_type = other RNA
gctagatcaagaagtacaggagg

SEQ ID NO: 191 - RNA, 23 nt, synthetic construct
  source 1..23: mol_type = other RNA
tatatccagggactctccagtgg

SEQ ID NO: 192 - RNA, 23 nt, synthetic construct
  source 1..23: mol_type = other RNA
gaagtacaggaggagactcaggg

SEQ ID NO: 193 - RNA, 23 nt, synthetic construct
  source 1..23: mol_type = other RNA
agaagtacaggaggagactcagg

SEQ ID NO: 194 - RNA, 23 nt, synthetic construct
  source 1..23: mol_type = other RNA
catggttactggttccgggaagg

SEQ ID NO: 195 - RNA, 23 nt, synthetic construct
  source 1..23: mol_type = other RNA
caagctagatcaagaagtacagg

SEQ ID NO: 196 - RNA, 23 nt, synthetic construct
  source 1..23: mol_type = other RNA
agggcagattccgcctccttggg

SEQ ID NO: 197 - RNA, 23 nt, synthetic construct
  source 1..23: mol_type = other RNA
cagttcatggttactggttccgg

SEQ ID NO: 198 - RNA, 23 nt, synthetic construct
  source 1..23: mol_type = other RNA
gggcagattccgcctccttgggg

SEQ ID NO: 199 - RNA, 23 nt, synthetic construct
  source 1..23: mol_type = other RNA
agttcatggttactggttccggg

SEQ ID NO: 200 - RNA, 23 nt, synthetic construct
  source 1..23: mol_type = other RNA
cagggcagattccgcctccttgg

SEQ ID NO: 201 - RNA, 23 nt, synthetic construct
  source 1..23: mol_type = other RNA
agggagcagttgttcctactggg

SEQ ID NO: 202 - RNA, 23 nt, synthetic construct
  source 1..23: mol_type = other RNA
gggagatttgtaactgtatttgg

SEQ ID NO: 203 - RNA, 23 nt, synthetic construct
  source 1..23: mol_type = other RNA
cagggagcagttgttcctactgg

SEQ ID NO: 204 - RNA, 23 nt, synthetic construct
  source 1..23: mol_type = other RNA
tgttcctactgggatccccaagg

SEQ ID NO: 205 - RNA, 23 nt, synthetic construct
  source 1..23: mol_type = other RNA
tgaaccattatccctcctcctgg

SEQ ID NO: 206 - RNA, 23 nt, synthetic construct
  source 1..23: mol_type = other RNA
tcctactgggatccccaaggagg

SEQ ID NO: 207 - RNA, 23 nt, synthetic construct
  source 1..23: mol_type = other RNA
tcctggcgtctacgatgctcagg

SEQ ID NO: 208 - RNA, 23 nt, synthetic construct
  source 1..23: mol_type = other RNA
tactgggatccccaaggaggcgg

SEQ ID NO: 209 - RNA, 23 nt, synthetic construct
  source 1..23: mol_type = other RNA
cctggcgtctacgatgctcaggg

SEQ ID NO: 210 - RNA, 23 nt, synthetic construct
  source 1..23: mol_type = other RNA
tgtcacatgcacagagagctggg

SEQ ID NO: 211 - RNA, 23 nt, synthetic construct
  source 1..23: mol_type = other RNA
gtcacatgcacagagagctgggg

SEQ ID NO: 212 - RNA, 23 nt, synthetic construct
  source 1..23: mol_type = other RNA
ctgtcacatgcacagagagctgg

SEQ ID NO: 213 - RNA, 23 nt, synthetic construct
  source 1..23: mol_type = other RNA
gggagatttgtaactgtatttgg

SEQ ID NO: 214 - RNA, 23 nt, synthetic construct
  source 1..23: mol_type = other RNA
tgtcacatgcacagagagctggg

SEQ ID NO: 215 - RNA, 23 nt, synthetic construct
  source 1..23: mol_type = other RNA
gtcacatgcacagagagctgggg

SEQ ID NO: 216 - RNA, 23 nt, synthetic construct
  source 1..23: mol_type = other RNA
ctgtcacatgcacagagagctgg

SEQ ID NO: 217 - RNA, 23 nt, synthetic construct
  source 1..23: mol_type = other RNA
tggttcatacttctttcggatgg

SEQ ID NO: 218 - RNA, 23 nt, synthetic construct
  source 1..23: mol_type = other RNA
gacgccaggaggagggataatgg

SEQ ID NO: 219 - RNA, 23 nt, synthetic construct
  source 1..23: mol_type = other RNA
gcatcgtagacgccaggaggagg

SEQ ID NO: 220 - RNA, 23 nt, synthetic construct
  source 1..23: mol_type = other RNA
ccctgagcatcgtagacgccagg

SEQ ID NO: 221 - RNA, 23 nt, synthetic construct
  source 1..23: mol_type = other RNA
catcgtagacgccaggaggaggg

SEQ ID NO: 222 - RNA, 23 nt, synthetic construct
  source 1..23: mol_type = other RNA
tgagcatcgtagacgccaggagg

SEQ ID NO: 223 - RNA, 23 nt, synthetic construct
  source 1..23: mol_type = other RNA
ataatggttcatacttctttcgg

SEQ ID NO: 224 - RNA, 23 nt, synthetic construct
  source 1..23: mol_type = other RNA
tacttctttcggatggagagagg

SEQ ID NO: 225 - RNA, 23 nt, synthetic construct
  source 1..23: mol_type = other RNA
gtacccatgaacttcccttgcgg

SEQ ID NO: 226 - RNA, 23 nt, synthetic construct
  source 1..23: mol_type = other RNA
tgtcacatgcacagagagctggg

SEQ ID NO: 227 - RNA, 23 nt, synthetic construct
  source 1..23: mol_type = other RNA
gtcacatgcacagagagctgggg

SEQ ID NO: 228 - RNA, 23 nt, synthetic construct
  source 1..23: mol_type = other RNA
ctgtcacatgcacagagagctgg

SEQ ID NO: 229 - RNA, 20 nt, synthetic construct
  source 1..20: mol_type = other RNA
aggttcgtgattggtgggtt

SEQ ID NO: 230 - RNA, 20 nt, synthetic construct
  source 1..20: mol_type = other RNA
tccttaggttcgtgattggt

SEQ ID NO: 231 - RNA, 20 nt, synthetic construct
  source 1..20: mol_type = other RNA
ttcatccttaggttcgtgat

SEQ ID NO: 232 - RNA, 20 nt, synthetic construct
  source 1..20: mol_type = other RNA
ggttcgtgattggtgggttt

SEQ ID NO: 233 - RNA, 20 nt, synthetic construct
  source 1..20: mol_type = other RNA
ccttaggttcgtgattggtg

SEQ ID NO: 234 - RNA, 20 nt, synthetic construct
  source 1..20: mol_type = other RNA
tcatccttaggttcgtgatt

SEQ ID NO: 235 - RNA, 20 nt, synthetic construct
  source 1..20: mol_type = other RNA
gttcgtgattggtgggtttg

SEQ ID NO: 236 - RNA, 22 nt, synthetic construct
  source 1..22: mol_type = other RNA
gtgattggtgggtttggatctt

SEQ ID NO: 237 - RNA, 22 nt, synthetic construct
  source 1..22: mol_type = other RNA
ttcgtgattggtgggtttggat

SEQ ID NO: 238 - RNA, 22 nt, synthetic construct
  source 1..22: mol_type = other RNA
gttcgtgattggtgggtttgga

SEQ ID NO: 239 - RNA, 22 nt, synthetic construct
  source 1..22: mol_type = other RNA
taggttcgtgattggtgggttt

SEQ ID NO: 240 - RNA, 20 nt, synthetic construct
  source 1..20: mol_type = other RNA
atccttaggttcgtgattgg

SEQ ID NO: 241 - RNA, 20 nt, synthetic construct
  source 1..20: mol_type = other RNA
acccaccaatcacgaaccta

SEQ ID NO: 242 - RNA, 20 nt, synthetic construct
  source 1..20: mol_type = other RNA
atcacgaacctaaggatgaa

SEQ ID NO: 243 - RNA, 20 nt, synthetic construct
  source 1..20: mol_type = other RNA
accaatcacgaacctaagga

SEQ ID NO: 244 - RNA, 20 nt, synthetic construct
  source 1..20: mol_type = other RNA
cccaccaatcacgaacctaa

SEQ ID NO: 245 - RNA, 20 nt, synthetic construct
  source 1..20: mol_type = other RNA
agatccaaacccaccaatca

SEQ ID NO: 246 - RNA, 20 nt, synthetic construct
  source 1..20: mol_type = other RNA
aatcacgaacctaaggatga

SEQ ID NO: 247 - RNA, 20 nt, synthetic construct
  source 1..20: mol_type = other RNA
caatcacgaacctaaggatg

SEQ ID NO: 248 - RNA, 20 nt, synthetic construct
  source 1..20: mol_type = other RNA
ccaatcacgaacctaaggat

SEQ ID NO: 249 - RNA, 20 nt, synthetic construct
  source 1..20: mol_type = other RNA
ccaccaatcacgaacctaag

SEQ ID NO: 250 - RNA, 20 nt, synthetic construct
  source 1..20: mol_type = other RNA
aacccaccaatcacgaacct

SEQ ID NO: 251 - RNA, 20 nt, synthetic construct
  source 1..20: mol_type = other RNA
aaacccaccaatcacgaacc

SEQ ID NO: 252 - RNA, 20 nt, synthetic construct
  source 1..20: mol_type = other RNA
atccaaacccaccaatcacg

SEQ ID NO: 253 - RNA, 20 nt, synthetic construct
  source 1..20: mol_type = other RNA
gatccaaacccaccaatcac

SEQ ID NO: 254 - RNA, 20 nt, synthetic construct
  source 1..20: mol_type = other RNA
gaagatccaaacccaccaat

SEQ ID NO: 255 - RNA, 22 nt, synthetic construct
  source 1..22: mol_type = other RNA
accaatcacgaacctaaggatg

SEQ ID NO: 256 - RNA, 22 nt, synthetic construct
  source 1..22: mol_type = other RNA
aagatccaaacccaccaatcac

SEQ ID NO: 257 - RNA, 22 nt, synthetic construct
  source 1..22: mol_type = other RNA
gaagatccaaacccaccaatca

SEQ ID NO: 258 - RNA, 22 nt, synthetic construct
  source 1..22: mol_type = other RNA
ccaccaatcacgaacctaagga

SEQ ID NO: 259 - RNA, 22 nt, synthetic construct
  source 1..22: mol_type = other RNA
caaacccaccaatcacgaacct

SEQ ID NO: 260 - RNA, 22 nt, synthetic construct
  source 1..22: mol_type = other RNA
atccaaacccaccaatcacgaa

SEQ ID NO: 261 - RNA, 22 nt, synthetic construct
  source 1..22: mol_type = other RNA
caccaatcacgaacctaaggat

SEQ ID NO: 262 - RNA, 22 nt, synthetic construct
  source 1..22: mol_type = other RNA
aacccaccaatcacgaacctaa

SEQ ID NO: 263 - RNA, 20 nt, synthetic construct
  source 1..20: mol_type = other RNA
gatccaaacccaccaatcac

SEQ ID NO: 264 - RNA, 20 nt, synthetic construct
  source 1..20: mol_type = other RNA
aggttcgtgattggtgggtt

SEQ ID NO: 265 - RNA, 20 nt, synthetic construct
  source 1..20: mol_type = other RNA
tccttaggttcgtgattggt

SEQ ID NO: 266 - RNA, 20 nt, synthetic construct
  source 1..20: mol_type = other RNA
atccttaggttcgtgattgg

SEQ ID NO: 267 - RNA, 20 nt, synthetic construct
  source 1..20: mol_type = other RNA
ttcatccttaggttcgtgat

SEQ ID NO: 268 - RNA, 20 nt, synthetic construct
  source 1..20: mol_type = other RNA
ccttaggttcgtgattggtg

SEQ ID NO: 269 - RNA, 20 nt, synthetic construct
  source 1..20: mol_type = other RNA
tcatccttaggttcgtgatt

SEQ ID NO: 270 - RNA, 20 nt, synthetic construct
  source 1..20: mol_type = other RNA
tgctttcatccttaggttcg

SEQ ID NO: 271 - RNA, 20 nt, synthetic construct
  source 1..20: mol_type = other RNA
tttgctttcatccttaggtt

SEQ ID NO: 272 - RNA, 20 nt, synthetic construct
  source 1..20: mol_type = other RNA
agcctttgctttcatcctta

SEQ ID NO: 273 - RNA, 20 nt, synthetic construct
  source 1..20: mol_type = other RNA
gctttcatccttaggttcgt

SEQ ID NO: 274 - RNA, 22 nt, synthetic construct
  source 1..22: mol_type = other RNA
taggttcgtgattggtgggttt

SEQ ID NO: 275 - RNA, 22 nt, synthetic construct
  source 1..22: mol_type = other RNA
tgagcctttgctttcatcctta

SEQ ID NO: 276 - RNA, 20 nt, synthetic construct
  source 1..20: mol_type = other RNA
atccttaggttcgtgattgg

SEQ ID NO: 277 - RNA, 20 nt, synthetic construct
  source 1..20: mol_type = other RNA
gaacctaaggatgaaagcaa

SEQ ID NO: 278 - RNA, 20 nt, synthetic construct
  source 1..20: mol_type = other RNA
acccaccaatcacgaaccta

SEQ ID NO: 279 - RNA, 20 nt, synthetic construct
  source 1..20: mol_type = other RNA
aacctaaggatgaaagcaaa

SEQ ID NO: 280 - RNA, 20 nt, synthetic construct
  source 1..20: mol_type = other RNA
atcacgaacctaaggatgaa

SEQ ID NO: 281 - RNA, 20 nt, synthetic construct
  source 1..20: mol_type = other RNA
accaatcacgaacctaagga

SEQ ID NO: 282 - RNA, 20 nt, synthetic construct
  source 1..20: mol_type = other RNA
cccaccaatcacgaacctaa

SEQ ID NO: 283 - RNA, 20 nt, synthetic construct
  source 1..20: mol_type = other RNA
cgaacctaaggatgaaagca

SEQ ID NO: 284 - RNA, 20 nt, synthetic construct
  source 1..20: mol_type = other RNA
acgaacctaaggatgaaagc

SEQ ID NO: 285 - RNA, 20 nt, synthetic construct
  source 1..20: mol_type = other RNA
cacgaacctaaggatgaaag

SEQ ID NO: 286 - RNA, 20 nt, synthetic construct
  source 1..20: mol_type = other RNA
aatcacgaacctaaggatga

SEQ ID NO: 287 - RNA, 20 nt, synthetic construct
  source 1..20: mol_type = other RNA
caatcacgaacctaaggatg

SEQ ID NO: 288 - RNA, 20 nt, synthetic construct
  source 1..20: mol_type = other RNA
ccaccaatcacgaacctaag

SEQ ID NO: 289 - RNA, 20 nt, synthetic construct
  source 1..20: mol_type = other RNA
aacccaccaatcacgaacct

SEQ ID NO: 290 - RNA, 20 nt, synthetic construct
  source 1..20: mol_type = other RNA
aaacccaccaatcacgaacc

SEQ ID NO: 291 - RNA, 22 nt, synthetic construct
  source 1..22: mol_type = other RNA
cacgaacctaaggatgaaagca

SEQ ID NO: 292 - RNA, 22 nt, synthetic construct
  source 1..22: mol_type = other RNA
accaatcacgaacctaaggatg

SEQ ID NO: 293 - RNA, 22 nt, synthetic construct
  source 1..22: mol_type = other RNA
acctaaggatgaaagcaaaggc

SEQ ID NO: 294 - RNA, 22 nt, synthetic construct
  source 1..22: mol_type = other RNA
cgaacctaaggatgaaagcaaa

SEQ ID NO: 295 - RNA, 22 nt, synthetic construct
  source 1..22: mol_type = other RNA
gaacctaaggatgaaagcaaag

SEQ ID NO: 296 - RNA, 22 nt, synthetic construct
  source 1..22: mol_type = other RNA
caaacccaccaatcacgaacct

SEQ ID NO: 297 - RNA, 22 nt, synthetic construct
  source 1..22: mol_type = other RNA
aacccaccaatcacgaacctaa

SEQ ID NO: 298 - RNA, 22 nt, synthetic construct
  source 1..22: mol_type = other RNA
caccaatcacgaacctaaggat

SEQ ID NO: 299 - RNA, 20 nt, synthetic construct
  source 1..20: mol_type = other RNA
ttcatccttaggttcgtgat

SEQ ID NO: 300 - RNA, 20 nt, synthetic construct
  source 1..20: mol_type = other RNA
gagcctttgctttcatcctt

SEQ ID NO: 301 - RNA, 20 nt, synthetic construct
  source 1..20: mol_type = other RNA
tgctttcatccttaggttcg

SEQ ID NO: 302 - RNA, 20 nt, synthetic construct
  source 1..20: mol_type = other RNA
tttgctttcatccttaggtt

SEQ ID NO: 303 - RNA, 20 nt, synthetic construct
  source 1..20: mol_type = other RNA
agcctttgctttcatcctta

SEQ ID NO: 304 - RNA, 20 nt, synthetic construct
  source 1..20: mol_type = other RNA
gctttcatccttaggttcgt

SEQ ID NO: 305 - RNA, 20 nt, synthetic construct
  source 1..20: mol_type = other RNA
tgagcctttgctttcatcct

SEQ ID NO: 306 - RNA, 22 nt, synthetic construct
  source 1..22: mol_type = other RNA
tgagcctttgctttcatcctta

SEQ ID NO: 307 - RNA, 22 nt, synthetic construct
  source 1..22: mol_type = other RNA
gagcctttgctttcatccttag

SEQ ID NO: 308 - RNA, 20 nt, synthetic construct
  source 1..20: mol_type = other RNA
ctttcatccttaggttcgtg

SEQ ID NO: 309 - RNA, 20 nt, synthetic construct
  source 1..20: mol_type = other RNA
gaacctaaggatgaaagcaa

SEQ ID NO: 310 - RNA, 20 nt, synthetic construct
  source 1..20: mol_type = other RNA
gatgaaagcaaaggctcagc

SEQ ID NO: 311 - RNA, 20 nt, synthetic construct
  source 1..20: mol_type = other RNA
aaggatgaaagcaaaggctc

SEQ ID NO: 312 - RNA, 20 nt, synthetic construct
  source 1..20: mol_type = other RNA
aacctaaggatgaaagcaaa

SEQ ID NO: 313 - RNA, 20 nt, synthetic construct
  source 1..20: mol_type = other RNA
atcacgaacctaaggatgaa

SEQ ID NO: 314 - RNA, 20 nt, synthetic construct
  source 1..20: mol_type = other RNA
accaatcacgaacctaagga

SEQ ID NO: 315 - RNA, 20 nt, synthetic construct
  source 1..20: mol_type = other RNA
ggatgaaagcaaaggctcag

SEQ ID NO: 316 - RNA, 20 nt, synthetic construct
  source 1..20: mol_type = other RNA
taaggatgaaagcaaaggct

SEQ ID NO: 317 - RNA, 20 nt, synthetic construct
  source 1..20: mol_type = other RNA
cgaacctaaggatgaaagca

SEQ ID NO: 318 - RNA, 20 nt, synthetic construct
  source 1..20: mol_type = other RNA
acgaacctaaggatgaaagc

SEQ ID NO: 319 - RNA, 20 nt, synthetic construct
  source 1..20: mol_type = other RNA
aatcacgaacctaaggatga

SEQ ID NO: 320 - RNA, 20 nt, synthetic construct
  source 1..20: mol_type = other RNA
caatcacgaacctaaggatg

SEQ ID NO: 321 - RNA, 20 nt, synthetic construct
  source 1..20: mol_type = other RNA
ccaatcacgaacctaaggat

SEQ ID NO: 322 - RNA, 22 nt, synthetic construct
  source 1..22: mol_type = other RNA
cacgaacctaaggatgaaagca

SEQ ID NO: 323 - RNA, 22 nt, synthetic construct
  source 1..22: mol_type = other RNA
accaatcacgaacctaaggatg

SEQ ID NO: 324 - RNA, 22 nt, synthetic construct
  source 1..22: mol_type = other RNA
atgaaagcaaaggctcagcagt

SEQ ID NO: 325 - RNA, 22 nt, synthetic construct
  source 1..22: mol_type = other RNA
gatgaaagcaaaggctcagcag

SEQ ID NO: 326 - RNA, 22 nt, synthetic construct
  source 1..22: mol_type = other RNA
acctaaggatgaaagcaaaggc

SEQ ID NO: 327 - RNA, 22 nt, synthetic construct
  source 1..22: mol_type = other RNA
cgaacctaaggatgaaagcaaa

SEQ ID NO: 328 - RNA, 22 nt, synthetic construct
  source 1..22: mol_type = other RNA
gaacctaaggatgaaagcaaag

SEQ ID NO: 329 - RNA, 22 nt, synthetic construct
  source 1..22: mol_type = other RNA
ccaccaatcacgaacctaagga

SEQ ID NO: 330 - RNA, 22 nt, synthetic construct
  source 1..22: mol_type = other RNA
caccaatcacgaacctaaggat

SEQ ID NO: 331 - RNA, 20 nt, synthetic construct
  source 1..20: mol_type = other RNA
taaggtcccaggtcaactgc

SEQ ID NO: 332 - RNA, 20 nt, synthetic construct
  source 1..20: mol_type = other RNA
caggtcaactgctgagcctt

SEQ ID NO: 333 - RNA, 20 nt, synthetic construct
  source 1..20: mol_type = other RNA
aggtcccaggtcaactgctg

SEQ ID NO: 334 - RNA, 20 nt, synthetic construct
  source 1..20: mol_type = other RNA
tgttaaggtcccaggtcaac

SEQ ID NO: 335 - RNA, 20 nt, synthetic construct
  source 1..20: mol_type = other RNA
acatttctgttaaggtccca

SEQ ID NO: 336 - RNA, 20 nt, synthetic construct
  source 1..20: mol_type = other RNA
aaggtcccaggtcaactgct

SEQ ID NO: 337 - RNA, 20 nt, synthetic construct
  source 1..20: mol_type = other RNA
ttctgttaaggtcccaggtc

SEQ ID NO: 338 - RNA, 22 nt, synthetic construct
  source 1..22: mol_type = other RNA
tttctgttaaggtcccaggtca

SEQ ID NO: 339 - RNA, 22 nt, synthetic construct
  source 1..22: mol_type = other RNA
caggtcaactgctgagcctttg

SEQ ID NO: 340 - RNA, 22 nt, synthetic construct
  source 1..22: mol_type = other RNA
gtcccaggtcaactgctgagcc

SEQ ID NO: 341 - RNA, 22 nt, synthetic construct
  source 1..22: mol_type = other RNA
ttaaggtcccaggtcaactgct

SEQ ID NO: 342 - RNA, 22 nt, synthetic construct
  source 1..22: mol_type = other RNA
gttaaggtcccaggtcaactgc

SEQ ID NO: 343 - RNA, 22 nt, synthetic construct
  source 1..22: mol_type = other RNA
tttctgttaaggtcccaggtca

SEQ ID NO: 344 - RNA, 22 nt, synthetic construct
  source 1..22: mol_type = other RNA
acatttctgttaaggtcccagg

SEQ ID NO: 345 - RNA, 20 nt, synthetic construct
  source 1..20: mol_type = other RNA
tgttaaggtcccaggtcaac

SEQ ID NO: 346 - RNA, 20 nt, synthetic construct
  source 1..20: mol_type = other RNA
gtctttaacacactcgatat

SEQ ID NO: 347 - RNA, 20 nt, synthetic construct
  source 1..20: mol_type = other RNA
ctcgatatcggtcacatttc

SEQ ID NO: 348 - RNA, 20 nt, synthetic construct
  source 1..20: mol_type = other RNA
aacacactcgatatcggtca

SEQ ID NO: 349 - RNA, 20 nt, synthetic construct
  source 1..20: mol_type = other RNA
ttaacacactcgatatcggt

SEQ ID NO: 350 - RNA, 20 nt, synthetic construct
  source 1..20: mol_type = other RNA
ggcgtctttaacacactcga

SEQ ID NO: 351 - RNA, 20 nt, synthetic construct
  source 1..20: mol_type = other RNA
tcggcgtctttaacacactc

SEQ ID NO: 352 - RNA, 22 nt, synthetic construct
  source 1..22: mol_type = other RNA
gcgtctttaacacactcgatat

SEQ ID NO: 353 - RNA, 22 nt, synthetic construct
  source 1..22: mol_type = other RNA
taacacactcgatatcggtcac

SEQ ID NO: 354 - RNA, 22 nt, synthetic construct
  source 1..22: mol_type = other RNA
gtctttaacacactcgatatcg

SEQ ID NO: 355 - RNA, 22 nt, synthetic construct
  source 1..22: mol_type = other RNA
gtcggcgtctttaacacactcg

SEQ ID NO: 356 - RNA, 22 nt, synthetic construct
  source 1..22: mol_type = other RNA
acacactcgatatcggtcacat

SEQ ID NO: 357 - RNA, 22 nt, synthetic construct
  source 1..22: mol_type = other RNA
acacactcgatatcggtcacat

SEQ ID NO: 358 - RNA, 20 nt, synthetic construct
  source 1..20: mol_type = other RNA
acacactcgatatcggtcac

SEQ ID NO: 359 - RNA, 20 nt, synthetic construct
  source 1..20: mol_type = other RNA
atcgagtgtgttaaagacgc

SEQ ID NO: 360 - RNA, 20 nt, synthetic construct
  source 1..20: mol_type = other RNA
gatatcgagtgtgttaaaga

SEQ ID NO: 361 - RNA, 20 nt, synthetic construct
  source 1..20: mol_type = other RNA
accgatatcgagtgtgttaa

SEQ ID NO: 362 - RNA, 20 nt, synthetic construct
  source 1..20: mol_type = other RNA
aatgtgaccgatatcgagtg

SEQ ID NO: 363 - RNA, 20 nt, synthetic construct
  source 1..20: mol_type = other RNA
gaaatgtgaccgatatcgag

SEQ ID NO: 364 - RNA, 20 nt, synthetic construct
  source 1..20: mol_type = other RNA
agtgtgttaaagacgccgac

SEQ ID NO: 365 - RNA, 20 nt, synthetic construct
  source 1..20: mol_type = other RNA
tcgagtgtgttaaagacgcc

SEQ ID NO: 366 - RNA, 20 nt, synthetic construct
  source 1..20: mol_type = other RNA
ccgatatcgagtgtgttaaa

SEQ ID NO: 367 - RNA, 20 nt, synthetic construct
  source 1..20: mol_type = other RNA
gaccgatatcgagtgtgtta

SEQ ID NO: 368 - RNA, 20 nt, synthetic construct
  source 1..20: mol_type = other RNA
tgaccgatatcgagtgtgtt

SEQ ID NO: 369 - RNA, 22 nt, synthetic construct
  source 1..22: mol_type = other RNA
gaccgatatcgagtgtgttaaa

SEQ ID NO: 370 - RNA, 22 nt, synthetic construct
  source 1..22: mol_type = other RNA
cgagtgtgttaaagacgccgac

SEQ ID NO: 371 - RNA, 22 nt, synthetic construct
  source 1..22: mol_type = other RNA
gatatcgagtgtgttaaagacg

SEQ ID NO: 372 - RNA, 22 nt, synthetic construct
  source 1..22: mol_type = other RNA
accgatatcgagtgtgttaaag

SEQ ID NO: 373 - RNA, 22 nt, synthetic construct
  source 1..22: mol_type = other RNA
gtgaccgatatcgagtgtgtta

SEQ ID NO: 374 - RNA, 22 nt, synthetic construct
  source 1..22: mol_type = other RNA
cgatatcgagtgtgttaaagac

SEQ ID NO: 375 - RNA, 22 nt, synthetic construct
  source 1..22: mol_type = other RNA
aaatgtgaccgatatcgagtgt

SEQ ID NO: 376 - RNA, 22 nt, synthetic construct
  source 1..22: mol_type = other RNA
acagaaatgtgaccgatatcga

SEQ ID NO: 377 - RNA, 20 nt, synthetic construct
  source 1..20: mol_type = other RNA
gtctttaacacactcgatat

SEQ ID NO: 378 - RNA, 20 nt, synthetic construct
  source 1..20: mol_type = other RNA
tctttaacacactcgatatc

SEQ ID NO: 379 - RNA, 20 nt, synthetic construct
  source 1..20: mol_type = other RNA
gtcggcgtctttaacacact

SEQ ID NO: 380 - RNA, 20 nt, synthetic construct
  source 1..20: mol_type = other RNA
aacacactcgatatcggtca

SEQ ID NO: 381 - RNA, 20 nt, synthetic construct
  source 1..20: mol_type = other RNA
ttaacacactcgatatcggt

SEQ ID NO: 382 - RNA, 20 nt, synthetic construct
  source 1..20: mol_type = other RNA
ggcgtctttaacacactcga

SEQ ID NO: 383 - RNA, 20 nt, synthetic construct
  source 1..20: mol_type = other RNA
tcggcgtctttaacacactc

SEQ ID NO: 384 - RNA, 22 nt, synthetic construct
  source 1..22: mol_type = other RNA
gcgtctttaacacactcgatat

SEQ ID NO: 385 - RNA, 22 nt, synthetic construct
  source 1..22: mol_type = other RNA
taacacactcgatatcggtcac

SEQ ID NO: 386 - RNA, 22 nt, synthetic construct
  source 1..22: mol_type = other RNA
gtctttaacacactcgatatcg

SEQ ID NO: 387 - RNA, 22 nt, synthetic construct
  source 1..22: mol_type = other RNA
gtcggcgtctttaacacactcg

SEQ ID NO: 388 - RNA, 22 nt, synthetic construct
  source 1..22: mol_type = other RNA
acacactcgatatcggtcacat

SEQ ID NO: 389 - RNA, 22 nt, synthetic construct
  source 1..22: mol_type = other RNA
cggcgtctttaacacactcgat

SEQ ID NO: 390 - RNA, 22 nt, synthetic construct
  source 1..22: mol_type = other RNA
gaatagtcggcgtctttaacac

SEQ ID NO: 391 - RNA, 20 nt, synthetic construct
  source 1..20: mol_type = other RNA
acacactcgatatcggtcac

SEQ ID NO: 392 - RNA, 20 nt, synthetic construct
  source 1..20: mol_type = other RNA
atttaccggcatagaatagt

SEQ ID NO: 393 - RNA, 20 nt, synthetic construct
  source 1..20: mol_type = other RNA
gacgccgactattctatgcc

SEQ ID NO: 394 - RNA, 20 nt, synthetic construct
  source 1..20: mol_type = other RNA
taaagacgccgactattcta

SEQ ID NO: 395 - RNA, 20 nt, synthetic construct
  source 1..20: mol_type = other RNA
tattctatgccggtaaatca

SEQ ID NO: 396 - RNA, 20 nt, synthetic construct
  source 1..20: mol_type = other RNA
actattctatgccggtaaat

SEQ ID NO: 397 - RNA, 20 nt, synthetic construct
  source 1..20: mol_type = other RNA
ccgactattctatgccggta

SEQ ID NO: 398 - RNA, 20 nt, synthetic construct
  source 1..20: mol_type = other RNA
gccgactattctatgccggt

SEQ ID NO: 399 - RNA, 22 nt, synthetic construct
  source 1..22: mol_type = other RNA
acgccgactattctatgccggt

SEQ ID NO: 400 - RNA, 22 nt, synthetic construct
  source 1..22: mol_type = other RNA
tattctatgccggtaaatcata

SEQ ID NO: 401 - RNA, 22 nt, synthetic construct
  source 1..22: mol_type = other RNA
actattctatgccggtaaatca

SEQ ID NO: 402 - RNA, 22 nt, synthetic construct
  source 1..22: mol_type = other RNA
cgactattctatgccggtaaat

SEQ ID NO: 403 - RNA, 22 nt, synthetic construct
  source 1..22: mol_type = other RNA
tgttaaagacgccgactattct

SEQ ID NO: 404 - RNA, 22 nt, synthetic construct
  source 1..22: mol_type = other RNA
agacgccgactattctatgccg

SEQ ID NO: 405 - RNA, 22 nt, synthetic construct
  source 1..22: mol_type = other RNA
ttaaagacgccgactattctat

SEQ ID NO: 406 - RNA, 22 nt, synthetic construct
  source 1..22: mol_type = other RNA
gttaaagacgccgactattcta

SEQ ID NO: 407 - RNA, 20 nt, synthetic construct
  source 1..20: mol_type = other RNA
atttaccggcatagaatagt

SEQ ID NO: 408 - RNA, 20 nt, synthetic construct
  source 1..20: mol_type = other RNA
tttaccggcatagaatagtc

SEQ ID NO: 409 - RNA, 20 nt, synthetic construct
  source 1..20: mol_type = other RNA
atgatttaccggcatagaat

SEQ ID NO: 410 - RNA, 20 nt, synthetic construct
  source 1..20: mol_type = other RNA
aatagtcggcgtctttaaca

SEQ ID NO: 411 - RNA, 20 nt, synthetic construct
  source 1..20: mol_type = other RNA
agaatagtcggcgtctttaa

SEQ ID NO: 412 - RNA, 20 nt, synthetic construct
  source 1..20: mol_type = other RNA
atagaatagtcggcgtcttt

SEQ ID NO: 413 - RNA, 22 nt, synthetic construct
  source 1..22: mol_type = other RNA
cggcatagaatagtcggcgtct

SEQ ID NO: 414 - RNA, 22 nt, synthetic construct
  source 1..22: mol_type = other RNA
agaatagtcggcgtctttaaca

SEQ ID NO: 415 - RNA, 22 nt, synthetic construct
  source 1..22: mol_type = other RNA
atagaatagtcggcgtctttaa

SEQ ID NO: 416 - RNA, 22 nt, synthetic construct
  source 1..22: mol_type = other RNA
gcatagaatagtcggcgtcttt

SEQ ID NO: 417 - RNA, 22 nt, synthetic construct
  source 1..22: mol_type = other RNA
cggcatagaatagtcggcgtct

SEQ ID NO: 418 - RNA, 22 nt, synthetic construct
  source 1..22: mol_type = other RNA
atttaccggcatagaatagtcg

SEQ ID NO: 419 - RNA, 22 nt, synthetic construct
  source 1..22: mol_type = other RNA
accggcatagaatagtcggcgt

SEQ ID NO: 420 - RNA, 22 nt, synthetic construct
  source 1..22: mol_type = other RNA
gaatagtcggcgtctttaacac

SEQ ID NO: 421 - RNA, 20 nt, synthetic construct
  source 1..20: mol_type = other RNA
ggccactcggacggtgtagt

SEQ ID NO: 422 - RNA, 20 nt, synthetic construct
  source 1..20: mol_type = other RNA
tggtgggttggccactcgga

SEQ ID NO: 423 - RNA, 20 nt, synthetic construct
  source 1..20: mol_type = other RNA
gttggccactcggacggtgt

SEQ ID NO: 424 - RNA, 20 nt, synthetic construct
  source 1..20: mol_type = other RNA
tgggttggccactcggacgg

SEQ ID NO: 425 - RNA, 20 nt, synthetic construct
  source 1..20: mol_type = other RNA
ggtgggttggccactcggac

SEQ ID NO: 426 - RNA, 20 nt, synthetic construct
  source 1..20: mol_type = other RNA
gaatggtgggttggccactc

SEQ ID NO: 427 - RNA, 20 nt, synthetic construct
  source 1..20: mol_type = other RNA
gccactcggacggtgtagtt

SEQ ID NO: 428 - RNA, 20 nt, synthetic construct
  source 1..20: mol_type = other RNA
actcggacggtgtagttggt

SEQ ID NO: 429 - RNA, 20 nt, synthetic construct
  source 1..20: mol_type = other RNA
aatggtgggttggccactcg

SEQ ID NO: 430 - RNA, 22 nt, synthetic construct
  source 1..22: mol_type = other RNA
ctcggacggtgtagttggtcac

SEQ ID NO: 431 - RNA, 22 nt, synthetic construct
  source 1..22: mol_type = other RNA
cactcggacggtgtagttggtc

SEQ ID NO: 432 - RNA, 22 nt, synthetic construct
  source 1..22: mol_type = other RNA
ggccactcggacggtgtagttg

SEQ ID NO: 433 - RNA, 22 nt, synthetic construct
  source 1..22: mol_type = other RNA
ttggccactcggacggtgtagt

SEQ ID NO: 434 - RNA, 22 nt, synthetic construct
  source 1..22: mol_type = other RNA
tcggacggtgtagttggtcact

SEQ ID NO: 435 - RNA, 22 nt, synthetic construct
  source 1..22: mol_type = other RNA
ggttggccactcggacggtgta

SEQ ID NO: 436 - RNA, 20 nt, synthetic construct
  source 1..20: mol_type = other RNA
ccaacccaccattctccacg

SEQ ID NO: 437 - RNA, 20 nt, synthetic construct
  source 1..20: mol_type = other RNA
caacccaccattctccacgt

SEQ ID NO: 438 - RNA, 20 nt, synthetic construct
  source 1..20: mol_type = other RNA
ggccaacccaccattctcca

SEQ ID NO: 439 - RNA, 20 nt, synthetic construct
  source 1..20: mol_type = other RNA
aacccaccattctccacgtg

SEQ ID NO: 440 - RNA, 20 nt, synthetic construct
  source 1..20: mol_type = other RNA
gtggccaacccaccattctc

SEQ ID NO: 441 - RNA, 20 nt, synthetic construct
  source 1..20: mol_type = other RNA
cgtccgagtggccaacccac

SEQ ID NO: 442 - RNA, 20 nt, synthetic construct
  source 1..20: mol_type = other RNA
caccgtccgagtggccaacc

SEQ ID NO: 443 - RNA, 22 nt, synthetic construct
  source 1..22: mol_type = other RNA
ccaccattctccacgtggatcc

SEQ ID NO: 444 - RNA, 22 nt, synthetic construct
  source 1..22: mol_type = other RNA
aacccaccattctccacgtgga

SEQ ID NO: 445 - RNA, 22 nt, synthetic construct
  source 1..22: mol_type = other RNA
ccaacccaccattctccacgtg

SEQ ID NO: 446 - RNA, 22 nt, synthetic construct
  source 1..22: mol_type = other RNA
gccaacccaccattctccacgt

SEQ ID NO: 447 - RNA, 22 nt, synthetic construct
  source 1..22: mol_type = other RNA
ccgagtggccaacccaccattc

SEQ ID NO: 448 - RNA, 22 nt, synthetic construct
  source 1..22: mol_type = other RNA
gtccgagtggccaacccaccat

SEQ ID NO: 449 - RNA, 22 nt, synthetic construct
  source 1..22: mol_type = other RNA
gtccgagtggccaacccaccat

SEQ ID NO: 450 - RNA, 22 nt, synthetic construct
  source 1..22: mol_type = other RNA
gtccgagtggccaacccaccat

SEQ ID NO: 451 - RNA, 20 nt, synthetic construct
  source 1..20: mol_type = other RNA
ccacgtggagaatggtgggt

SEQ ID NO: 452 - RNA, 20 nt, synthetic construct
  source 1..20: mol_type = other RNA
agaatggtgggttggccact

SEQ ID NO: 453 - RNA, 20 nt, synthetic construct
  source 1..20: mol_type = other RNA
ggatccacgtggagaatggt

SEQ ID NO: 454 - RNA, 20 nt, synthetic construct
  source 1..20: mol_type = other RNA
aagaggatccacgtggagaa

SEQ ID NO: 455 - RNA, 20 nt, synthetic construct
  source 1..20: mol_type = other RNA
gaatggtgggttggccactc

SEQ ID NO: 456 - RNA, 20 nt, synthetic construct
  source 1..20: mol_type = other RNA
cacgtggagaatggtgggtt

SEQ ID NO: 457 - RNA, 20 nt, synthetic construct
  source 1..20: mol_type = other RNA
gatccacgtggagaatggtg

SEQ ID NO: 458 - RNA, 20 nt, synthetic construct
  source 1..20: mol_type = other RNA
agaggatccacgtggagaat

SEQ ID NO: 459 - RNA, 20 nt, synthetic construct
  source 1..20: mol_type = other RNA
aatggtgggttggccactcg

SEQ ID NO: 460 - RNA, 20 nt, synthetic construct
  source 1..20: mol_type = other RNA
gtggagaatggtgggttggc

SEQ ID NO: 461 - RNA, 22 nt, synthetic construct
  source 1..22: mol_type = other RNA
acgtggagaatggtgggttggc

SEQ ID NO: 462 - RNA, 22 nt, synthetic construct
  source 1..22: mol_type = other RNA
ggagaatggtgggttggccact

SEQ ID NO: 463 - RNA, 22 nt, synthetic construct
  source 1..22: mol_type = other RNA
ccacgtggagaatggtgggttg

SEQ ID NO: 464 - RNA, 22 nt, synthetic construct
  source 1..22: mol_type = other RNA
atccacgtggagaatggtgggt

SEQ ID NO: 465 - RNA, 22 nt, synthetic construct
  source 1..22: mol_type = other RNA
gatccacgtggagaatggtggg

SEQ ID NO: 466 - RNA, 22 nt, synthetic construct
  source 1..22: mol_type = other RNA
ggatccacgtggagaatggtgg

SEQ ID NO: 467 - RNA, 20 nt, synthetic construct
  source 1..20: mol_type = other RNA
ccacgtggagaatggtgggt

SEQ ID NO: 468 - RNA, 20 nt, synthetic construct
  source 1..20: mol_type = other RNA
ggatccacgtggagaatggt

SEQ ID NO: 469 - RNA, 20 nt, synthetic construct
  source 1..20: mol_type = other RNA
aagaggatccacgtggagaa

SEQ ID NO: 470 - RNA, 20 nt, synthetic construct
  source 1..20: mol_type = other RNA
aggatccacgtggagaatgg

SEQ ID NO: 471 - RNA, 20 nt, synthetic construct
  source 1..20: mol_type = other RNA
cacgtggagaatggtgggtt

SEQ ID NO: 472 - RNA, 20 nt, synthetic construct
  source 1..20: mol_type = other RNA
gatccacgtggagaatggtg

SEQ ID NO: 473 - RNA, 20 nt, synthetic construct
  source 1..20: mol_type = other RNA
agaggatccacgtggagaat

SEQ ID NO: 474 - RNA, 20 nt, synthetic construct
  source 1..20: mol_type = other RNA
gtggagaatggtgggttggc

SEQ ID NO: 475 - RNA, 20 nt, synthetic construct
  source 1..20: mol_type = other RNA
gggaagaggatccacgtgga

SEQ ID NO: 476 - RNA, 22 nt, synthetic construct
  source 1..22: mol_type = other RNA
acgtggagaatggtgggttggc

SEQ ID NO: 477 - RNA, 22 nt, synthetic construct
  source 1..22: mol_type = other RNA
ggagaatggtgggttggccact

SEQ ID NO: 478 - RNA, 22 nt, synthetic construct
  source 1..22: mol_type = other RNA
ccacgtggagaatggtgggttg

SEQ ID NO: 479 - RNA, 22 nt, synthetic construct
  source 1..22: mol_type = other RNA
atccacgtggagaatggtgggt

SEQ ID NO: 480 - RNA, 22 nt, synthetic construct
  source 1..22: mol_type = other RNA
gatccacgtggagaatggtggg

SEQ ID NO: 481 - RNA, 22 nt, synthetic construct
  source 1..22: mol_type = other RNA
ggatccacgtggagaatggtgg

SEQ ID NO: 482 - RNA, 20 nt, synthetic construct
  source 1..20: mol_type = other RNA
ccaacccaccattctccacg

SEQ ID NO: 483 - RNA, 20 nt, synthetic construct
  source 1..20: mol_type = other RNA
tccacgtggatcctcttccc

SEQ ID NO: 484 - RNA, 20 nt, synthetic construct
  source 1..20: mol_type = other RNA
ccacgtggatcctcttccct

SEQ ID NO: 485 - RNA, 20 nt, synthetic construct
  source 1..20: mol_type = other RNA
aacccaccattctccacgtg

SEQ ID NO: 486 - RNA, 20 nt, synthetic construct
  source 1..20: mol_type = other RNA
gtggccaacccaccattctc

SEQ ID NO: 487 - RNA, 22 nt, synthetic construct
  source 1..22: mol_type = other RNA
accattctccacgtggatcctc

SEQ ID NO: 488 - RNA, 22 nt, synthetic construct
  source 1..22: mol_type = other RNA
tccacgtggatcctcttccctg

SEQ ID NO: 489 - RNA, 22 nt, synthetic construct
  source 1..22: mol_type = other RNA
accattctccacgtggatcctc

SEQ ID NO: 490 - RNA, 22 nt, synthetic construct
  source 1..22: mol_type = other RNA
caccattctccacgtggatcct

SEQ ID NO: 491 - RNA, 22 nt, synthetic construct
  source 1..22: mol_type = other RNA
ccaccattctccacgtggatcc

SEQ ID NO: 492 - RNA, 22 nt, synthetic construct
  source 1..22: mol_type = other RNA
aacccaccattctccacgtgga

SEQ ID NO: 493 - RNA, 22 nt, synthetic construct
  source 1..22: mol_type = other RNA
ccaacccaccattctccacgtg

SEQ ID NO: 494 - RNA, 22 nt, synthetic construct
  source 1..22: mol_type = other RNA
gccaacccaccattctccacgt

SEQ ID NO: 495 - RNA, 22 nt, synthetic construct
  source 1..22: mol_type = other RNA
tctccacgtggatcctcttccc

SEQ ID NO: 496 - RNA, 22 nt, synthetic construct
  source 1..22: mol_type = other RNA
attctccacgtggatcctcttc

SEQ ID NO: 497 - RNA, 22 nt, synthetic construct
  source 1..22: mol_type = other RNA
cattctccacgtggatcctctt

SEQ ID NO: 498 - RNA, 20 nt, synthetic construct
  source 1..20: mol_type = other RNA
ccacgtggagaatggtgggt

SEQ ID NO: 499 - RNA, 20 nt, synthetic construct
  source 1..20: mol_type = other RNA
ggatccacgtggagaatggt

SEQ ID NO: 500 - RNA, 20 nt, synthetic construct
  source 1..20: mol_type = other RNA
aagaggatccacgtggagaa

SEQ ID NO: 501 - RNA, 20 nt, synthetic construct
  source 1..20: mol_type = other RNA
ctcagggaagaggatccacg

SEQ ID NO: 502 - RNA, 20 nt, synthetic construct
  source 1..20: mol_type = other RNA
cacgtggagaatggtgggtt

SEQ ID NO: 503 - RNA, 20 nt, synthetic construct
  source 1..20: mol_type = other RNA
gatccacgtggagaatggtg

SEQ ID NO: 504 - RNA, 20 nt, synthetic construct
  source 1..20: mol_type = other RNA
agaggatccacgtggagaat

SEQ ID NO: 505 - RNA, 20 nt, synthetic construct
  source 1..20: mol_type = other RNA
tcagggaagaggatccacgt

SEQ ID NO: 506 - RNA, 20 nt, synthetic construct
  source 1..20: mol_type = other RNA
ttctcagggaagaggatcca

SEQ ID NO: 507 - RNA, 20 nt, synthetic construct
  source 1..20: mol_type = other RNA
ggaagaggatccacgtggag

SEQ ID NO: 508 - RNA, 20 nt, synthetic construct
  source 1..20: mol_type = other RNA
gggaagaggatccacgtgga

SEQ ID NO: 509 - RNA, 20 nt, synthetic construct
  source 1..20: mol_type = other RNA
cagggaagaggatccacgtg

SEQ ID NO: 510 - RNA, 22 nt, synthetic construct
  source 1..22: mol_type = other RNA
acgtggagaatggtgggttggc

SEQ ID NO: 511 - RNA, 22 nt, synthetic construct
  source 1..22: mol_type = other RNA
ccacgtggagaatggtgggttg

SEQ ID NO: 512 - RNA, 22 nt, synthetic construct
  source 1..22: mol_type = other RNA
atccacgtggagaatggtgggt

SEQ ID NO: 513 - RNA, 22 nt, synthetic construct
  source 1..22: mol_type = other RNA
gatccacgtggagaatggtggg

SEQ ID NO: 514 - RNA, 22 nt, synthetic construct
  source 1..22: mol_type = other RNA
gttctcagggaagaggatccac

SEQ ID NO: 515 - RNA, 22 nt, synthetic construct
  source 1..22: mol_type = other RNA
ggatccacgtggagaatggtgg

SEQ ID NO: 516 - RNA, 22 nt, synthetic construct
  source 1..22: mol_type = other RNA
tgttctcagggaagaggatcca

SEQ ID NO: 517 - RNA, 20 nt, synthetic construct
  source 1..20: mol_type = other RNA
ccacgtggagaatggtgggt

SEQ ID NO: 518 - RNA, 20 nt, synthetic construct
  source 1..20: mol_type = other RNA
tgtgaagtgaccaactacac

SEQ ID NO: 519 - RNA, 20 nt, synthetic construct
  source 1..20: mol_type = other RNA
gaccaactacaccgtccgag

SEQ ID NO: 520 - RNA, 20 nt, synthetic construct
  source 1..20: mol_type = other RNA
aagtgaccaactacaccgtc

SEQ ID NO: 521 - RNA, 20 nt, synthetic construct
  source 1..20: mol_type = other RNA
gtgaccaactacaccgtccg

SEQ ID NO: 522 - RNA, 20 nt, synthetic construct
  source 1..20: mol_type = other RNA
agaatggtgggttggccact

SEQ ID NO: 523 - RNA, 20 nt, synthetic construct
  source 1..20: mol_type = other RNA
aagaggatccacgtggagaa

SEQ ID NO: 524 - RNA, 20 nt, synthetic construct
  source 1..20: mol_type = other RNA
ccaacccaccattctccacg

SEQ ID NO: 525 - RNA, 20 nt, synthetic construct
  source 1..20: mol_type = other RNA
catttttctcactgttctca

SEQ ID NO: 526 - RNA, 20 nt, synthetic construct
  source 1..20: mol_type = other RNA
aaagaaaaaagacacagcga

SEQ ID NO: 527 - RNA, 20 nt, synthetic construct
  source 1..20: mol_type = other RNA
aggatccacgtggagaatgg

SEQ ID NO: 528 - RNA, 20 nt, synthetic construct
  source 1..20: mol_type = other RNA
ggatccacgtggagaatggt

SEQ ID NO: 529 - RNA, 22 nt, synthetic construct
  source 1..22: mol_type = other RNA
tgaagtgaccaactacaccgtc

SEQ ID NO: 530 - RNA, 22 nt, synthetic construct
  source 1..22: mol_type = other RNA
ttatgtgaagtgaccaactaca

SEQ ID NO: 531 - RNA, 22 nt, synthetic construct
  source 1..22: mol_type = other RNA
tccacgtggagaatggtgggtt

SEQ ID NO: 532 - RNA, 20 nt, synthetic construct
  source 1..20: mol_type = other RNA
tcagggaagaggatccacgt

SEQ ID NO: 533 - RNA, 20 nt, synthetic construct
  source 1..20: mol_type = other RNA
ctcactgttctcagggaaga

SEQ ID NO: 534 - RNA, 20 nt, synthetic construct
  source 1..20: mol_type = other RNA
atttaccggcatagaatagt

SEQ ID NO: 535 - RNA, 20 nt, synthetic construct
  source 1..20: mol_type = other RNA
cttaacagaaatgtgaccga

SEQ ID NO: 536 - RNA, 20 nt, synthetic construct
  source 1..20: mol_type = other RNA
cagaaatgtgaccgatatcg

SEQ ID NO: 537 - RNA, 20 nt, synthetic construct
  source 1..20: mol_type = other RNA
gtcggcgtctttaacacact

SEQ ID NO: 538 - RNA, 20 nt, synthetic construct
  source 1..20: mol_type = other RNA
gaaatgtgaccgatatcgag

SEQ ID NO: 539 - RNA, 20 nt, synthetic construct
  source 1..20: mol_type = other RNA
caatagagagtatgatttac

SEQ ID NO: 540 - RNA, 20 nt, synthetic construct
  source 1..20: mol_type = other RNA
agagtatgatttaccggcat

SEQ ID NO: 541 - RNA, 20 nt, synthetic construct
  source 1..20: mol_type = other RNA
atgatttaccggcatagaat

SEQ ID NO: 542 - RNA, 20 nt, synthetic construct
  source 1..20: mol_type = other RNA
aacaccggcaaatacacgtg

SEQ ID NO: 543 - RNA, 20 nt, synthetic construct
  source 1..20: mol_type = other RNA
ccaacaccggcaaatacacg

SEQ ID NO: 544 - RNA, 20 nt, synthetic construct
  source 1..20: mol_type = other RNA
caccaacaccggcaaataca

SEQ ID NO: 545 - RNA, 20 nt, synthetic construct
  source 1..20: mol_type = other RNA
gccaccaacaccggcaaata

SEQ ID NO: 546 - RNA, 20 nt, synthetic construct
  source 1..20: mol_type = other RNA
aagccaccaacaccggcaaa

SEQ ID NO: 547 - RNA, 22 nt, synthetic construct
  source 1..22: mol_type = other RNA
aacaccggcaaatacacgtgca

SEQ ID NO: 548 - RNA, 22 nt, synthetic construct
  source 1..22: mol_type = other RNA
caccaacaccggcaaatacacg

SEQ ID NO: 549 - RNA, 22 nt, synthetic construct
  source 1..22: mol_type = other RNA
accaacaccggcaaatacacgt

SEQ ID NO: 550 - RNA, 22 nt, synthetic construct
  source 1..22: mol_type = other RNA
gccaccaacaccggcaaataca

SEQ ID NO: 551 - RNA, 22 nt, synthetic construct
  source 1..22: mol_type = other RNA
gcagaagccaccaacaccggca

SEQ ID NO: 552 - RNA, 22 nt, synthetic construct
  source 1..22: mol_type = other RNA
ccaacaccggcaaatacacgtg

SEQ ID NO: 553 - RNA, 22 nt, synthetic construct
  source 1..22: mol_type = other RNA
caacaccggcaaatacacgtgc

SEQ ID NO: 554 - RNA, 22 nt, synthetic construct
  source 1..22: mol_type = other RNA
caccggcaaatacacgtgcacc

SEQ ID NO: 555 - RNA, 20 nt, synthetic construct
  source 1..20: mol_type = other RNA
ccggcaaatacacgtgcacc

SEQ ID NO: 556 - RNA, 20 nt, synthetic construct
  source 1..20: mol_type = other RNA
accggcaaatacacgtgcac

SEQ ID NO: 557 - RNA, 20 nt, synthetic construct
  source 1..20: mol_type = other RNA
ggcaaatacacgtgcaccaa

SEQ ID NO: 558 - RNA, 20 nt, synthetic construct
  source 1..20: mol_type = other RNA
gcaaatacacgtgcaccaac

SEQ ID NO: 559 - RNA, 20 nt, synthetic construct
  source 1..20: mol_type = other RNA
caaatacacgtgcaccaaca

SEQ ID NO: 560 - RNA, 22 nt, synthetic construct
  source 1..22: mol_type = other RNA
ccggcaaatacacgtgcaccaa

SEQ ID NO: 561 - RNA, 22 nt, synthetic construct
  source 1..22: mol_type = other RNA
ggcaaatacacgtgcaccaaca

SEQ ID NO: 562 - RNA, 20 nt, synthetic construct
  source 1..20: mol_type = other RNA
tacacgtgcaccaacaaaca

SEQ ID NO: 563 - RNA, 20 nt, synthetic construct
  source 1..20: mol_type = other RNA
aatacacgtgcaccaacaaa

SEQ ID NO: 564 - RNA, 20 nt, synthetic construct
  source 1..20: mol_type = other RNA
acacgtgcaccaacaaacac

SEQ ID NO: 565 - RNA, 22 nt, synthetic construct
  source 1..22: mol_type = other RNA
aaatacacgtgcaccaacaaac

SEQ ID NO: 566 - RNA, 22 nt, synthetic construct
  source 1..22: mol_type = other RNA
acacgtgcaccaacaaacacgg

SEQ ID NO: 567 - RNA, 20 nt, synthetic construct
  source 1..20: mol_type = other RNA
attccatttatgtgtttgtt

SEQ ID NO: 568 - RNA, 20 nt, synthetic construct
  source 1..20: mol_type = other RNA
aattccatttatgtgtttgt

SEQ ID NO: 569 - RNA, 20 nt, synthetic construct
  source 1..20: mol_type = other RNA
agcaattccatttatgtgtt

SEQ ID NO: 570 - RNA, 20 nt, synthetic construct
  source 1..20: mol_type = other RNA
cttaagcaattccatttatg

SEQ ID NO: 571 - RNA, 20 nt, synthetic construct
  source 1..20: mol_type = other RNA
ggcttaagcaattccattta

SEQ ID NO: 572 - RNA, 20 nt, synthetic construct
  source 1..20: mol_type = other RNA
ataaatggaattgcttaagc

SEQ ID NO: 573 - RNA, 20 nt, synthetic construct
  source 1..20: mol_type = other RNA
aaatggaattgcttaagccg

SEQ ID NO: 574 - RNA, 20 nt, synthetic construct
  source 1..20: mol_type = other RNA
ggaattgcttaagccgtgtt

SEQ ID NO: 575 - RNA, 20 nt, synthetic construct
  source 1..20: mol_type = other RNA
acacataaatggaattgctt

SEQ ID NO: 576 - RNA, 20 nt, synthetic construct
  source 1..20: mol_type = other RNA
aacacataaatggaattgct

SEQ ID NO: 577 - RNA, 20 nt, synthetic construct
  source 1..20: mol_type = other RNA
cacataaatggaattgctta

SEQ ID NO: 578 - RNA, 22 nt, synthetic construct
  source 1..22: mol_type = other RNA
caaacacataaatggaattgct

SEQ ID NO: 579 - RNA, 22 nt, synthetic construct
  source 1..22: mol_type = other RNA
aaacacataaatggaattgctt

SEQ ID NO: 580 - RNA, 22 nt, synthetic construct
  source 1..22: mol_type = other RNA
caaacacataaatggaattgct

SEQ ID NO: 581 - RNA, 22 nt, synthetic construct
  source 1..22: mol_type = other RNA
caattccatttatgtgtttgtt

SEQ ID NO: 582 - RNA, 22 nt, synthetic construct
  source 1..22: mol_type = other RNA
gcaattccatttatgtgtttgt

SEQ ID NO: 583 - RNA, 22 nt, synthetic construct
  source 1..22: mol_type = other RNA
gcttaagcaattccatttatgt

SEQ ID NO: 584 - RNA, 20 nt, synthetic construct
  source 1..20: mol_type = other RNA
ttatgtgtttgttagaggta

SEQ ID NO: 585 - RNA, 20 nt, synthetic construct
  source 1..20: mol_type = other RNA
tttatgtgtttgttagaggt

SEQ ID NO: 586 - RNA, 20 nt, synthetic construct
  source 1..20: mol_type = other RNA
atttatgtgtttgttagagg

SEQ ID NO: 587 - RNA, 20 nt, synthetic construct
  source 1..20: mol_type = other RNA
ccatttatgtgtttgttaga

SEQ ID NO: 588 - RNA, 20 nt, synthetic construct
  source 1..20: mol_type = other RNA
tccatttatgtgtttgttag

SEQ ID NO: 589 - RNA, 20 nt, synthetic construct
  source 1..20: mol_type = other RNA
ttccatttatgtgtttgtta

SEQ ID NO: 590 - RNA, 20 nt, synthetic construct
  source 1..20: mol_type = other RNA
tctaacaaacacataaatgg

SEQ ID NO: 591 - RNA, 20 nt, synthetic construct
  source 1..20: mol_type = other RNA
ctctaacaaacacataaatg

SEQ ID NO: 592 - RNA, 20 nt, synthetic construct
  source 1..20: mol_type = other RNA
acctctaacaaacacataaa

SEQ ID NO: 593 - RNA, 22 nt, synthetic construct
  source 1..22: mol_type = other RNA
ctctaacaaacacataaatgga

SEQ ID NO: 594 - RNA, 22 nt, synthetic construct
  source 1..22: mol_type = other RNA
atttatgtgtttgttagaggta

SEQ ID NO: 595 - RNA, 22 nt, synthetic construct
  source 1..22: mol_type = other RNA
gcaattccatttatgtgtttgt

SEQ ID NO: 596 - RNA, 20 nt, synthetic construct
  source 1..20: mol_type = other RNA
ctcacagacccagaagtgac

SEQ ID NO: 597 - RNA, 20 nt, synthetic construct
  source 1..20: mol_type = other RNA
cctctcacagacccagaagt

SEQ ID NO: 598 - RNA, 20 nt, synthetic construct
  source 1..20: mol_type = other RNA
tcacagacccagaagtgacc

SEQ ID NO: 599 - RNA, 20 nt, synthetic construct
  source 1..20: mol_type = other RNA
tcctctcacagacccagaag

SEQ ID NO: 600 - RNA, 20 nt, synthetic construct
  source 1..20: mol_type = other RNA
tgtcctctcacagacccaga

SEQ ID NO: 601 - RNA, 20 nt, synthetic construct
  source 1..20: mol_type = other RNA
ctgtcctctcacagacccag

SEQ ID NO: 602 - RNA, 20 nt, synthetic construct
  source 1..20: mol_type = other RNA
gctgtcctctcacagaccca

SEQ ID NO: 603 - RNA, 20 nt, synthetic construct
  source 1..20: mol_type = other RNA
cgctgtcctctcacagaccc

SEQ ID NO: 604 - RNA, 20 nt, synthetic construct
  source 1..20: mol_type = other RNA
ccgctgtcctctcacagacc

SEQ ID NO: 605 - RNA, 22 nt, synthetic construct
  source 1..22: mol_type = other RNA
ctgtcctctcacagacccagaa

SEQ ID NO: 606 - RNA, 22 nt, synthetic construct
  source 1..22: mol_type = other RNA
cacagacccagaagtgaccaat

SEQ ID NO: 607 - RNA, 22 nt, synthetic construct
  source 1..22: mol_type = other RNA
tgtcctctcacagacccagaag

SEQ ID NO: 608 - RNA, 22 nt, synthetic construct
  source 1..22: mol_type = other RNA
tcctctcacagacccagaagtg

SEQ ID NO: 609 - RNA, 22 nt, synthetic construct
  source 1..22: mol_type = other RNA
gctgtcctctcacagacccaga

SEQ ID NO: 610 - RNA, 22 nt, synthetic construct
  source 1..22: mol_type = other RNA
tccgctgtcctctcacagaccc

SEQ ID NO: 611 - RNA, 19 nt, synthetic construct
  source 1..19: mol_type = other RNA
gtgaccaattattccctca

SEQ ID NO: 612 - RNA, 20 nt, synthetic construct
  source 1..20: mol_type = other RNA
gtgaccaattattccctcaa

SEQ ID NO: 613 - RNA, 20 nt, synthetic construct
  source 1..20: mol_type = other RNA
aagtgaccaattattccctc

SEQ ID NO: 614 - RNA, 20 nt, synthetic construct
  source 1..20: mol_type = other RNA
gtgaccaattattccctcaa

SEQ ID NO: 615 - RNA, 20 nt, synthetic construct
  source 1..20: mol_type = other RNA
tgaccaattattccctcaag

SEQ ID NO: 616 - RNA, 20 nt, synthetic construct
  source 1..20: mol_type = other RNA
gaccaattattccctcaagg

SEQ ID NO: 617 - RNA, 20 nt, synthetic construct
  source 1..20: mol_type = other RNA
gaagtgaccaattattccct

SEQ ID NO: 618 - RNA, 22 nt, synthetic construct
  source 1..22: mol_type = other RNA
cagacccagaagtgaccaatta

SEQ ID NO: 619 - RNA, 22 nt, synthetic construct
  source 1..22: mol_type = other RNA
gacccagaagtgaccaattatt

SEQ ID NO: 620 - RNA, 22 nt, synthetic construct
  source 1..22: mol_type = other RNA
acagacccagaagtgaccaatt

SEQ ID NO: 621 - RNA, 22 nt, synthetic construct
  source 1..22: mol_type = other RNA
gtgaccaattattccctcaagg

SEQ ID NO: 622 - RNA, 22 nt, synthetic construct
  source 1..22: mol_type = other RNA
tgaccaattattccctcaaggg

SEQ ID NO: 623 - RNA, 22 nt, synthetic construct
  source 1..22: mol_type = other RNA
agtgaccaattattccctcaag

SEQ ID NO: 624 - RNA, 20 nt, synthetic construct
  source 1..20: mol_type = other RNA
ccaggggaagcctcttccca

SEQ ID NO: 625 - RNA, 20 nt, synthetic construct
  source 1..20: mol_type = other RNA
caggggaagcctcttcccaa

SEQ ID NO: 626 - RNA, 20 nt, synthetic construct
  source 1..20: mol_type = other RNA
aggggaagcctcttcccaag

SEQ ID NO: 627 - RNA, 20 nt, synthetic construct
  source 1..20: mol_type = other RNA
gaagcctcttcccaaggact

SEQ ID NO: 628 - RNA, 20 nt, synthetic construct
  source 1..20: mol_type = other RNA
ccttgggaagaggcttcccc

SEQ ID NO: 629 - RNA, 20 nt, synthetic construct
  source 1..20: mol_type = other RNA
tgggaagaggcttcccctgg

SEQ ID NO: 630 - RNA, 20 nt, synthetic construct
  source 1..20: mol_type = other RNA
aggcttcccctggcacccct

SEQ ID NO: 631 - RNA, 20 nt, synthetic construct
  source 1..20: mol_type = other RNA
ggcttcccctggcacccctt

SEQ ID NO: 632 - RNA, 22 nt, synthetic construct
  source 1..22: mol_type = other RNA
tgccaggggaagcctcttccca

SEQ ID NO: 633 - RNA, 22 nt, synthetic construct
  source 1..22: mol_type = other RNA
ggggtgccaggggaagcctctt

SEQ ID NO: 634 - RNA, 22 nt, synthetic construct
  source 1..22: mol_type = other RNA
ttgggaagaggcttcccctggc

SEQ ID NO: 635 - RNA, 22 nt, synthetic construct
  source 1..22: mol_type = other RNA
cttgggaagaggcttcccctgg

SEQ ID NO: 636 - RNA, 22 nt, synthetic construct
  source 1..22: mol_type = other RNA
ccttgggaagaggcttcccctg

SEQ ID NO: 637 - RNA, 22 nt, synthetic construct
  source 1..22: mol_type = other RNA
agtccttgggaagaggcttccc

SEQ ID NO: 638 - RNA, 22 nt, synthetic construct
  source 1..22: mol_type = other RNA
aggcttcccctggcaccccttg

SEQ ID NO: 639 - RNA, 22 nt, synthetic construct
  source 1..22: mol_type = other RNA
ggcttcccctggcaccccttga

SEQ ID NO: 640 - RNA, 22 nt, synthetic construct
  source 1..22: mol_type = other RNA
aagaggcttcccctggcacccc

SEQ ID NO: 641 - RNA, 22 nt, synthetic construct
  source 1..22: mol_type = other RNA
agaggcttcccctggcacccct

SEQ ID NO: 642 - RNA, 20 nt, synthetic construct
  source 1..20: mol_type = other RNA
aaatataatagctggcatca

SEQ ID NO: 643 - RNA, 20 nt, synthetic construct
  source 1..20: mol_type = other RNA
tataatagctggcatcacgg

SEQ ID NO: 644 - RNA, 20 nt, synthetic construct
  source 1..20: mol_type = other RNA
aatataatagctggcatcac

SEQ ID NO: 645 - RNA, 20 nt, synthetic construct
  source 1..20: mol_type = other RNA
ataatagctggcatcacggt

SEQ ID NO: 646 - RNA, 20 nt, synthetic construct
  source 1..20: mol_type = other RNA
agaaatataatagctggcat

SEQ ID NO: 647 - RNA, 20 nt, synthetic construct
  source 1..20: mol_type = other RNA
aggagaaatataatagctgg

SEQ ID NO: 648 - RNA, 20 nt, synthetic construct
  source 1..20: mol_type = other RNA
gccagctattatatttctcc

SEQ ID NO: 649 - RNA, 20 nt, synthetic construct
  source 1..20: mol_type = other RNA
caccgtgatgccagctatta

SEQ ID NO: 650 - RNA, 20 nt, synthetic construct
  source 1..20: mol_type = other RNA
cagctattatatttctcctg

SEQ ID NO: 651 - RNA, 20 nt, synthetic construct
  source 1..20: mol_type = other RNA
agctattatatttctcctgt

SEQ ID NO: 652 - RNA, 22 nt, synthetic construct
  source 1..22: mol_type = other RNA
tataatagctggcatcacggtg

SEQ ID NO: 653 - RNA, 22 nt, synthetic construct
  source 1..22: mol_type = other RNA
aaatataatagctggcatcacg

SEQ ID NO: 654 - RNA, 22 nt, synthetic construct
  source 1..22: mol_type = other RNA
caggagaaatataatagctggc

SEQ ID NO: 655 - RNA, 22 nt, synthetic construct
  source 1..22: mol_type = other RNA
tacaggagaaatataatagctg

SEQ ID NO: 656 - RNA, 22 nt, synthetic construct
  source 1..22: mol_type = other RNA
taatagctggcatcacggtgac

SEQ ID NO: 657 - RNA, 22 nt, synthetic construct
  source 1..22: mol_type = other RNA
tagctggcatcacggtgacttc

SEQ ID NO: 658 - RNA, 22 nt, synthetic construct
  source 1..22: mol_type = other RNA
ggagaaatataatagctggcat

SEQ ID NO: 659 - RNA, 22 nt, synthetic construct
  source 1..22: mol_type = other RNA
ccgtgatgccagctattatatt

SEQ ID NO: 660 - RNA, 22 nt, synthetic construct
  source 1..22: mol_type = other RNA
accgtgatgccagctattatat

SEQ ID NO: 661 - RNA, 22 nt, synthetic construct
  source 1..22: mol_type = other RNA
tcaccgtgatgccagctattat

SEQ ID NO: 662 - RNA, 20 nt, synthetic construct
  source 1..20: mol_type = other RNA
agctggcatcacggtgactt

SEQ ID NO: 663 - RNA, 20 nt, synthetic construct
  source 1..20: mol_type = other RNA
gctggcatcacggtgacttc

SEQ ID NO: 664 - RNA, 20 nt, synthetic construct
  source 1..20: mol_type = other RNA
ggcatcacggtgacttcaat

SEQ ID NO: 665 - RNA, 22 nt, synthetic construct
  source 1..22: mol_type = other RNA
ggcatcacggtgacttcaatta

SEQ ID NO: 666 - RNA, 22 nt, synthetic construct
  source 1..22: mol_type = other RNA
gcatcacggtgacttcaattat

SEQ ID NO: 667 - RNA, 20 nt, synthetic construct
  source 1..20: mol_type = other RNA
tgacttcaattatgaacgtc

SEQ ID NO: 668 - RNA, 20 nt, synthetic construct
  source 1..20: mol_type = other RNA
ttatgaacgtcaggcaacgt

SEQ ID NO: 669 - RNA, 20 nt, synthetic construct
  source 1..20: mol_type = other RNA
caattatgaacgtcaggcaa

SEQ ID NO: 670 - RNA, 20 nt, synthetic construct
  source 1..20: mol_type = other RNA
gacttcaattatgaacgtca

SEQ ID NO: 671 - RNA, 20 nt, synthetic construct
  source 1..20: mol_type = other RNA
acggtgacttcaattatgaa

SEQ ID NO: 672 - RNA, 20 nt, synthetic construct
  source 1..20: mol_type = other RNA
cataattgaagtcaccgtga

SEQ ID NO: 673 - RNA, 20 nt, synthetic construct
  source 1..20: mol_type = other RNA
gttcataattgaagtcaccg

SEQ ID NO: 674 - RNA, 20 nt, synthetic construct
  source 1..20: mol_type = other RNA
acgttcataattgaagtcac

SEQ ID NO: 675 - RNA, 20 nt, synthetic construct
  source 1..20: mol_type = other RNA
tgcctgacgttcataattga

SEQ ID NO: 676 - RNA, 20 nt, synthetic construct
  source 1..20: mol_type = other RNA
cgttgcctgacgttcataat

SEQ ID NO: 677 - RNA, 20 nt, synthetic construct
  source 1..20: mol_type = other RNA
ttcaattatgaacgtcaggc

SEQ ID NO: 678 - RNA, 20 nt, synthetic construct
  source 1..20: mol_type = other RNA
cttcaattatgaacgtcagg

SEQ ID NO: 679 - RNA, 20 nt, synthetic construct
  source 1..20: mol_type = other RNA
gtgacttcaattatgaacgt

SEQ ID NO: 680 - RNA, 20 nt, synthetic construct
  source 1..20: mol_type = other RNA
tcacggtgacttcaattatg

SEQ ID NO: 681 - RNA, 20 nt, synthetic construct
  source 1..20: mol_type = other RNA
ttcataattgaagtcaccgt

SEQ ID NO: 682 - RNA, 20 nt, synthetic construct
  source 1..20: mol_type = other RNA
ctgacgttcataattgaagt

SEQ ID NO: 683 - RNA, 20 nt, synthetic construct
  source 1..20: mol_type = other RNA
ttgcctgacgttcataattg

SEQ ID NO: 684 - RNA, 20 nt, synthetic construct
  source 1..20: mol_type = other RNA
gttgcctgacgttcataatt

SEQ ID NO: 685 - RNA, 22 nt, synthetic construct
  source 1..22: mol_type = other RNA
caattatgaacgtcaggcaacg

SEQ ID NO: 686 - RNA, 22 nt, synthetic construct
  source 1..22: mol_type = other RNA
tatgaacgtcaggcaacgttga

SEQ ID NO: 687 - RNA, 22 nt, synthetic construct
  source 1..22: mol_type = other RNA
tgacttcaattatgaacgtcag

SEQ ID NO: 688 - RNA, 22 nt, synthetic construct
  source 1..22: mol_type = other RNA
cggtgacttcaattatgaacgt

SEQ ID NO: 689 - RNA, 22 nt, synthetic construct
  source 1..22: mol_type = other RNA
gttcataattgaagtcaccgtg

SEQ ID NO: 690 - RNA, 22 nt, synthetic construct
  source 1..22: mol_type = other RNA
cataattgaagtcaccgtgatg

SEQ ID NO: 691 - RNA, 22 nt, synthetic construct
  source 1..22: mol_type = other RNA
cgttcataattgaagtcaccgt

SEQ ID NO: 692 - RNA, 22 nt, synthetic construct
  source 1..22: mol_type = other RNA
tgcctgacgttcataattgaag

SEQ ID NO: 693 - RNA, 22 nt, synthetic construct
  source 1..22: mol_type = other RNA
ttgcctgacgttcataattgaa

SEQ ID NO: 694 - RNA, 22 nt, synthetic construct
  source 1..22: mol_type = other RNA
cgttgcctgacgttcataattg

SEQ ID NO: 695 - RNA, 22 nt, synthetic construct
  source 1..22: mol_type = other RNA
tcaattatgaacgtcaggcaac

SEQ ID NO: 696 - RNA, 22 nt, synthetic construct
  source 1..22: mol_type = other RNA
attatgaacgtcaggcaacgtt

SEQ ID NO: 697 - RNA, 22 nt, synthetic construct
  source 1..22: mol_type = other RNA
acggtgacttcaattatgaacg

SEQ ID NO: 698 - RNA, 22 nt, synthetic construct
  source 1..22: mol_type = other RNA
cacggtgacttcaattatgaac

SEQ ID NO: 699 - RNA, 22 nt, synthetic construct
  source 1..22: mol_type = other RNA
tcataattgaagtcaccgtgat

SEQ ID NO: 700 - RNA, 22 nt, synthetic construct
  source 1..22: mol_type = other RNA
acgttcataattgaagtcaccg

SEQ ID NO: 701 - RNA, 22 nt, synthetic construct
  source 1..22: mol_type = other RNA
gcctgacgttcataattgaagt

SEQ ID NO: 702 - RNA, 20 nt, synthetic construct
  source 1..20: mol_type = other RNA
ggatcagcaaatgtcacaac

SEQ ID NO: 703 - RNA, 20 nt, synthetic construct
  source 1..20: mol_type = other RNA
tggatcagcaaatgtcacaa

SEQ ID NO: 704 - RNA, 20 nt, synthetic construct
  source 1..20: mol_type = other RNA
tttggatcagcaaatgtcac

SEQ ID NO: 705 - RNA, 20 nt, synthetic construct
  source 1..20: mol_type = other RNA
ttttggatcagcaaatgtca

SEQ ID NO: 706 - RNA, 20 nt, synthetic construct
  source 1..20: mol_type = other RNA
acttttggatcagcaaatgt

SEQ ID NO: 707 - RNA, 20 nt, synthetic construct
  source 1..20: mol_type = other RNA
aatacttttggatcagcaaa

SEQ ID NO: 708 - RNA, 20 nt, synthetic construct
  source 1..20: mol_type = other RNA
ataatacttttggatcagca

SEQ ID NO: 709 - RNA, 20 nt, synthetic construct
  source 1..20: mol_type = other RNA
aataatacttttggatcagc

SEQ ID NO: 710 - RNA, 20 nt, synthetic construct
  source 1..20: mol_type = other RNA
caataatacttttggatcag

SEQ ID NO: 711 - RNA, 19 nt, synthetic construct
  source 1..19: mol_type = other RNA
gctgatccaaaagtattat

SEQ ID NO: 712 - RNA, 20 nt, synthetic construct
  source 1..20: mol_type = other RNA
tgatccaaaagtattattgg

SEQ ID NO: 713 - RNA, 20 nt, synthetic construct
  source 1..20: mol_type = other RNA
gctgatccaaaagtattatt

SEQ ID NO: 714 - RNA, 20 nt, synthetic construct
  source 1..20: mol_type = other RNA
atttgctgatccaaaagtat

SEQ ID NO: 715 - RNA, 20 nt, synthetic construct
  source 1..20: mol_type = other RNA
gacatttgctgatccaaaag

SEQ ID NO: 716 - RNA, 20 nt, synthetic construct
  source 1..20: mol_type = other RNA
gtgacatttgctgatccaaa

SEQ ID NO: 717 - RNA, 20 nt, synthetic construct
  source 1..20: mol_type = other RNA
tgtgacatttgctgatccaa

SEQ ID NO: 718 - RNA, 20 nt, synthetic construct
  source 1..20: mol_type = other RNA
ttgtgacatttgctgatcca

SEQ ID NO: 719 - RNA, 20 nt, synthetic construct
  source 1..20: mol_type = other RNA
gttgtgacatttgctgatcc

SEQ ID NO: 720 - RNA, 20 nt, synthetic construct
  source 1..20: mol_type = other RNA
tgttgtgacatttgctgatc

SEQ ID NO: 721 - RNA, 22 nt, synthetic construct
  source 1..22: mol_type = other RNA
atttgctgatccaaaagtatta

SEQ ID NO: 722 - RNA, 22 nt, synthetic construct
  source 1..22: mol_type = other RNA
ctgatccaaaagtattattggc

SEQ ID NO: 723 - RNA, 22 nt, synthetic construct
  source 1..22: mol_type = other RNA
aataatacttttggatcagcaa

SEQ ID NO: 724 - RNA, 22 nt, synthetic construct
  source 1..22: mol_type = other RNA
ttttggatcagcaaatgtcaca

SEQ ID NO: 725 - RNA, 22 nt, synthetic construct
  source 1..22: mol_type = other RNA
tttggatcagcaaatgtcacaa

SEQ ID NO: 726 - RNA, 22 nt, synthetic construct
  source 1..22: mol_type = other RNA
tacttttggatcagcaaatgtc

SEQ ID NO: 727 - RNA, 22 nt, synthetic construct
  source 1..22: mol_type = other RNA
taatacttttggatcagcaaat

SEQ ID NO: 728 - RNA, 22 nt, synthetic construct
  source 1..22: mol_type = other RNA
ctgatccaaaagtattattggc

SEQ ID NO: 729 - RNA, 22 nt, synthetic construct
  source 1..22: mol_type = other RNA
gatccaaaagtattattggcat

SEQ ID NO: 730 - RNA, 22 nt, synthetic construct
  source 1..22: mol_type = other RNA
tcagcaaatgtcacaacaacct

SEQ ID NO: 731 - RNA, 22 nt, synthetic construct
  source 1..22: mol_type = other RNA
atacttttggatcagcaaatgt

SEQ ID NO: 732 - RNA, 22 nt, synthetic construct
  source 1..22: mol_type = other RNA
aatacttttggatcagcaaatg

SEQ ID NO: 733 - RNA, 22 nt, synthetic construct
  source 1..22: mol_type = other RNA
ataatacttttggatcagcaaa

SEQ ID NO: 734 - RNA, 22 nt, synthetic construct
  source 1..22: mol_type = other RNA
tttgctgatccaaaagtattat

SEQ ID NO: 735 - RNA, 22 nt, synthetic construct
  source 1..22: mol_type = other RNA
tgctgatccaaaagtattattg

SEQ ID NO: 736 - RNA, 22 nt, synthetic construct
  source 1..22: mol_type = other RNA
gttgtgacatttgctgatccaa

SEQ ID NO: 737 - RNA, 22 nt, synthetic construct
  source 1..22: mol_type = other RNA
acatttgctgatccaaaagtat

SEQ ID NO: 738 - RNA, 22 nt, synthetic construct
  source 1..22: mol_type = other RNA
ggatcagcaaatgtcacaacaa

SEQ ID NO: 739 - RNA, 20 nt, synthetic construct
  source 1..20: mol_type = other RNA
agcaaatgtcacaacaacct

SEQ ID NO: 740 - RNA, 20 nt, synthetic construct
  source 1..20: mol_type = other RNA
gtcacaacaaccttggaagt

SEQ ID NO: 741 - RNA, 20 nt, synthetic construct
  source 1..20: mol_type = other RNA
cacaacaaccttggaagtag

SEQ ID NO: 742 - RNA, 20 nt, synthetic construct
  source 1..20: mol_type = other RNA
tgtcacaacaaccttggaag

SEQ ID NO: 743 - RNA, 20 nt, synthetic construct
  source 1..20: mol_type = other RNA
aatgtcacaacaaccttgga

SEQ ID NO: 744 - RNA, 20 nt, synthetic construct
  source 1..20: mol_type = other RNA
aaatgtcacaacaaccttgg

SEQ ID NO: 745 - RNA, 20 nt, synthetic construct
  source 1..20: mol_type = other RNA
caaatgtcacaacaaccttg

SEQ ID NO: 746 - RNA, 20 nt, synthetic construct
  source 1..20: mol_type = other RNA
gcaaatgtcacaacaacctt

SEQ ID NO: 747 - RNA, 20 nt, synthetic construct
  source 1..20: mol_type = other RNA
ggatcagcaaatgtcacaac

SEQ ID NO: 748 - RNA, 22 nt, synthetic construct
  source 1..22: mol_type = other RNA
cacaacaaccttggaagtagta

SEQ ID NO: 749 - RNA, 22 nt, synthetic construct
  source 1..22: mol_type = other RNA
gcaaatgtcacaacaaccttgg

SEQ ID NO: 750 - RNA, 22 nt, synthetic construct
  source 1..22: mol_type = other RNA
tgtcacaacaaccttggaagta

SEQ ID NO: 751 - RNA, 22 nt, synthetic construct
  source 1..22: mol_type = other RNA
atgtcacaacaaccttggaagt

SEQ ID NO: 752 - RNA, 19 nt, synthetic construct
  source 1..19: mol_type = other RNA
caacaaccttggaagtagt

SEQ ID NO: 753 - RNA, 20 nt, synthetic construct
  source 1..20: mol_type = other RNA
caacaaccttggaagtagta

SEQ ID NO: 754 - RNA, 20 nt, synthetic construct
  source 1..20: mol_type = other RNA
acaaccttggaagtagtagg

SEQ ID NO: 755 - RNA, 20 nt, synthetic construct
  source 1..20: mol_type = other RNA
caaccttggaagtagtaggt

SEQ ID NO: 756 - RNA, 20 nt, synthetic construct
  source 1..20: mol_type = other RNA
aaccttggaagtagtaggta

SEQ ID NO: 757 - RNA, 22 nt, synthetic construct
  source 1..22: mol_type = other RNA
caaccttggaagtagtaggtaa

SEQ ID NO: 758 - RNA, 22 nt, synthetic construct
  source 1..22: mol_type = other RNA
acaaccttggaagtagtaggta

SEQ ID NO: 759 - RNA, 23 nt, synthetic construct
  source 1..23: mol_type = other RNA
ctgatccgggctttgtcaaatgg

SEQ ID NO: 760 - RNA, 23 nt, synthetic construct
  source 1..23: mol_type = other RNA
tacacgtgcaccaacaaacacgg

SEQ ID NO: 761 - RNA, 23 nt, synthetic construct
  source 1..23: mol_type = other RNA
caaatggacttttgagatcctgg

SEQ ID NO: 762 - RNA, 23 nt, synthetic construct
  source 1..23: mol_type = other RNA
gaatgaatggatcacggaaaagg

SEQ ID NO: 763 - RNA, 23 nt, synthetic construct
  source 1..23: mol_type = other RNA
aaggcagaagccaccaacaccgg

SEQ ID NO: 764 - RNA, 23 nt, synthetic construct
  source 1..23: mol_type = other RNA
atgagaataagcagaatgaatgg

SEQ ID NO: 765 - RNA, 23 nt, synthetic construct
  source 1..23: mol_type = other RNA
taagcagaatgaatggatcacgg

SEQ ID NO: 766 - RNA, 23 nt, synthetic construct
  source 1..23: mol_type = other RNA
attgcttaagccgtgtttgttgg

SEQ ID NO: 767 - RNA, 23 nt, synthetic construct
  source 1..23: mol_type = other RNA
tgtcatccaaaattaagagcagg

SEQ ID NO: 768 - RNA, 23 nt, synthetic construct
  source 1..23: mol_type = other RNA
gttggtgcacgtgtatttgccgg

SEQ ID NO: 769 - RNA, 23 nt, synthetic construct
  source 1..23: mol_type = other RNA
acctctaacaaacacataaatgg

SEQ ID NO: 770 - RNA, 23 nt, synthetic construct
  source 1..23: mol_type = other RNA
ttgttgaccgctccttgtatggg

SEQ ID NO: 771 - RNA, 23 nt, synthetic construct
  source 1..23: mol_type = other RNA
cttgttgaccgctccttgtatgg

SEQ ID NO: 772 - RNA, 23 nt, synthetic construct
  source 1..23: mol_type = other RNA
gaaagaagacaacgacacgctgg

SEQ ID NO: 773 - RNA, 23 nt, synthetic construct
  source 1..23: mol_type = other RNA
ttattccctcaaggggtgccagg

SEQ ID NO: 774 - RNA, 23 nt, synthetic construct
  source 1..23: mol_type = other RNA
tgaccaattattccctcaagggg

SEQ ID NO: 775 - RNA, 23 nt, synthetic construct
  source 1..23: mol_type = other RNA
tattccctcaaggggtgccaggg

SEQ ID NO: 776 - RNA, 23 nt, synthetic construct
  source 1..23: mol_type = other RNA
gtgaccaattattccctcaaggg

SEQ ID NO: 777 - RNA, 23 nt, synthetic construct
  source 1..23: mol_type = other RNA
attccctcaaggggtgccagggg

SEQ ID NO: 778 - RNA, 23 nt, synthetic construct
  source 1..23: mol_type = other RNA
ttgatcatgatgcccgccttggg

SEQ ID NO: 779 - RNA, 23 nt, synthetic construct
  source 1..23: mol_type = other RNA
atgcagacagagccgatggtagg

SEQ ID NO: 780 - RNA, 23 nt, synthetic construct
  source 1..23: mol_type = other RNA
tgatcatgatgcccgccttgggg

SEQ ID NO: 781 - RNA, 23 nt, synthetic construct
  source 1..23: mol_type = other RNA
tttgatcatgatgcccgccttgg

SEQ ID NO: 782 - RNA, 23 nt, synthetic construct
  source 1..23: mol_type = other RNA
gcaccccttgagggaataattgg

SEQ ID NO: 783 - RNA, 23 nt, synthetic construct
  source 1..23: mol_type = other RNA
aacaatgcagacagagccgatgg

SEQ ID NO: 784 - RNA, 23 nt, synthetic construct
  source 1..23: mol_type = other RNA
gggaataattggtcacttctggg

SEQ ID NO: 785 - RNA, 23 nt, synthetic construct
  source 1..23: mol_type = other RNA
agggaataattggtcacttctgg

SEQ ID NO: 786 - RNA, 23 nt, synthetic construct
  source 1..23: mol_type = other RNA
aggaataaacctcaagtccttgg

SEQ ID NO: 787 - RNA, 23 nt, synthetic construct
  source 1..23: mol_type = other RNA
atgatgcccgccttggggtcagg

SEQ ID NO: 788 - RNA, 23 nt, synthetic construct
  source 1..23: mol_type = other RNA
cttcccctggcaccccttgaggg

SEQ ID NO: 789 - RNA, 23 nt, synthetic construct
  source 1..23: mol_type = other RNA
gcttcccctggcaccccttgagg

SEQ ID NO: 790 - RNA, 23 nt, synthetic construct
  source 1..23: mol_type = other RNA
aacctcaagtccttgggaagagg

SEQ ID NO: 791 - RNA, 23 nt, synthetic construct
  source 1..23: mol_type = other RNA
ggaataaacctcaagtccttggg

SEQ ID NO: 792 - RNA, 23 nt, synthetic construct
  source 1..23: mol_type = other RNA
aaaccagcagactaaactacagg

SEQ ID NO: 793 - RNA, 23 nt, synthetic construct
  source 1..23: mol_type = other RNA
tacaggagaaatataatagctgg

SEQ ID NO: 794 - RNA, 23 nt, synthetic construct
  source 1..23: mol_type = other RNA
aaatataatagctggcatcacgg

SEQ ID NO: 795 - RNA, 23 nt, synthetic construct
  source 1..23: mol_type = other RNA
gattctgaatataaattatatgg

SEQ ID NO: 796 - RNA, 23 nt, synthetic construct
  source 1..23: mol_type = other RNA
tgctgatccaaaagtattattgg

SEQ ID NO: 797 - RNA, 23 nt, synthetic construct
  source 1..23: mol_type = other RNA
tcagcgagagttaatgattctgg

SEQ ID NO: 798 - RNA, 23 nt, synthetic construct
  source 1..23: mol_type = other RNA
tgacttcaattatgaacgtcagg

SEQ ID NO: 799 - RNA, 23 nt, synthetic construct
  source 1..23: mol_type = other RNA
tgttatgccaataatacttttgg

SEQ ID NO: 800 - RNA, 23 nt, synthetic construct
  source 1..23: mol_type = other RNA
gtatttacctactacttccaagg

SEQ ID NO: 801 - RNA, 23 nt, synthetic construct
  source 1..23: mol_type = other RNA
taatttaaacattcccatagagg

SEQ ID NO: 802 - RNA, 20 nt, synthetic construct
  source 1..20: mol_type = other RNA
taagtgatgatgtccaaaca

SEQ ID NO: 803 - RNA, 20 nt, synthetic construct
  source 1..20: mol_type = other RNA
tgatgatgtccaaacatggc

SEQ ID NO: 804 - RNA, 20 nt, synthetic construct
  source 1..20: mol_type = other RNA
aacatggcaggagagtaaaa

SEQ ID NO: 805 - RNA, 20 nt, synthetic construct
  source 1..20: mol_type = other RNA
atgtttggacatcatcactt

SEQ ID NO: 806 - RNA, 20 nt, synthetic construct
  source 1..20: mol_type = other RNA
ttttactctcctgccatgtt

SEQ ID NO: 807 - RNA, 20 nt, synthetic construct
  source 1..20: mol_type = other RNA
aggacagctgttatttccta

SEQ ID NO: 808 - RNA, 20 nt, synthetic construct
  source 1..20: mol_type = other RNA
gacagctgttatttcctaag

SEQ ID NO: 809 - RNA, 20 nt, synthetic construct
  source 1..20: mol_type = other RNA
agctgttatttcctaagtga

SEQ ID NO: 810 - RNA, 20 nt, synthetic construct
  source 1..20: mol_type = other RNA
tgttatttcctaagtgatga

SEQ ID NO: 811 - RNA, 20 nt, synthetic construct
  source 1..20: mol_type = other RNA
tgatgtccaaacatggcagg

SEQ ID NO: 812 - RNA, 20 nt, synthetic construct
  source 1..20: mol_type = other RNA
atgtccaaacatggcaggag

SEQ ID NO: 813 - RNA, 20 nt, synthetic construct
  source 1..20: mol_type = other RNA
ggcaggagagtaaaatggcc

SEQ ID NO: 814 - RNA, 20 nt, synthetic construct
  source 1..20: mol_type = other RNA
caggagagtaaaatggcctg

SEQ ID NO: 815 - RNA, 20 nt, synthetic construct
  source 1..20: mol_type = other RNA
ggaaataacagctgtcctta

SEQ ID NO: 816 - RNA, 20 nt, synthetic construct
  source 1..20: mol_type = other RNA
atcacttaggaaataacagc

SEQ ID NO: 817 - RNA, 20 nt, synthetic construct
  source 1..20: mol_type = other RNA
atcatcacttaggaaataac

SEQ ID NO: 818 - RNA, 20 nt, synthetic construct
  source 1..20: mol_type = other RNA
gccattttactctcctgcca

SEQ ID NO: 819 - RNA, 20 nt, synthetic construct
  source 1..20: mol_type = other RNA
taaggacagctgttatttcc

SEQ ID NO: 820 - RNA, 20 nt, synthetic construct
  source 1..20: mol_type = other RNA
aaggacagctgttatttcct

SEQ ID NO: 821 - RNA, 20 nt, synthetic construct
  source 1..20: mol_type = other RNA
acagctgttatttcctaagt

SEQ ID NO: 822 - RNA, 20 nt, synthetic construct
  source 1..20: mol_type = other RNA
gctgttatttcctaagtgat

SEQ ID NO: 823 - RNA, 20 nt, synthetic construct
  source 1..20: mol_type = other RNA
atttcctaagtgatgatgtc

SEQ ID NO: 824 - RNA, 20 nt, synthetic construct
  source 1..20: mol_type = other RNA
ttcctaagtgatgatgtcca

SEQ ID NO: 825 - RNA, 20 nt, synthetic construct
  source 1..20: mol_type = other RNA
cctaagtgatgatgtccaaa

SEQ ID NO: 826 - RNA, 20 nt, synthetic construct
  source 1..20: mol_type = other RNA
atgatgtccaaacatggcag

SEQ ID NO: 827 - RNA, 20 nt, synthetic construct
  source 1..20: mol_type = other RNA
gatgtccaaacatggcagga

SEQ ID NO: 828 - RNA, 20 nt, synthetic construct
  source 1..20: mol_type = other RNA
gtccaaacatggcaggagag

SEQ ID NO: 829 - RNA, 20 nt, synthetic construct
  source 1..20: mol_type = other RNA
tccaaacatggcaggagagt

SEQ ID NO: 830 - RNA, 20 nt, synthetic construct
  source 1..20: mol_type = other RNA
caaacatggcaggagagtaa

SEQ ID NO: 831 - RNA, 19 nt, synthetic construct
  source 1..19: mol_type = other RNA
gctgtccttatgccaaatc

SEQ ID NO: 832 - RNA, 20 nt, synthetic construct
  source 1..20: mol_type = other RNA
taacagctgtccttatgcca

SEQ ID NO: 833 - RNA, 20 nt, synthetic construct
  source 1..20: mol_type = other RNA
ataacagctgtccttatgcc

SEQ ID NO: 834 - RNA, 20 nt, synthetic construct
  source 1..20: mol_type = other RNA
aataacagctgtccttatgc

SEQ ID NO: 835 - RNA, 20 nt, synthetic construct
  source 1..20: mol_type = other RNA
catcatcacttaggaaataa

SEQ ID NO: 836 - RNA, 20 nt, synthetic construct
  source 1..20: mol_type = other RNA
gacatcatcacttaggaaat

SEQ ID NO: 837 - RNA, 20 nt, synthetic construct
  source 1..20: mol_type = other RNA
ttggacatcatcacttagga

SEQ ID NO: 838 - RNA, 20 nt, synthetic construct
  source 1..20: mol_type = other RNA
tttggacatcatcacttagg

SEQ ID NO: 839 - RNA, 20 nt, synthetic construct
  source 1..20: mol_type = other RNA
gtttggacatcatcacttag

SEQ ID NO: 840 - RNA, 20 nt, synthetic construct
  source 1..20: mol_type = other RNA
ctgccatgtttggacatcat

SEQ ID NO: 841 - RNA, 20 nt, synthetic construct
  source 1..20: mol_type = other RNA
ctcctgccatgtttggacat

SEQ ID NO: 842 - RNA, 20 nt, synthetic construct
  source 1..20: mol_type = other RNA
actctcctgccatgtttgga

SEQ ID NO: 843 - RNA, 20 nt, synthetic construct
  source 1..20: mol_type = other RNA
ttactctcctgccatgtttg

SEQ ID NO: 844 - RNA, 20 nt, synthetic construct
  source 1..20: mol_type = other RNA
aggccattttactctcctgc

SEQ ID NO: 845 - RNA, 22 nt, synthetic construct
  source 1..22: mol_type = other RNA
agctgttatttcctaagtgatg

SEQ ID NO: 846 - RNA, 22 nt, synthetic construct
  source 1..22: mol_type = other RNA
agctgttatttcctaagtgatg

SEQ ID NO: 847 - RNA, 22 nt, synthetic construct
  source 1..22: mol_type = other RNA
tttggcataaggacagctgtta

SEQ ID NO: 848 - RNA, 22 nt, synthetic construct
  source 1..22: mol_type = other RNA
ttaggaaataacagctgtcctt

SEQ ID NO: 849 - RNA, 22 nt, synthetic construct
  source 1..22: mol_type = other RNA
cttaggaaataacagctgtcct

SEQ ID NO: 850 - RNA, 22 nt, synthetic construct
  source 1..22: mol_type = other RNA
tcatcacttaggaaataacagc

SEQ ID NO: 851 - RNA, 22 nt, synthetic construct
  source 1..22: mol_type = other RNA
tctcctgccatgtttggacatc

SEQ ID NO: 852 - RNA, 22 nt, synthetic construct
  source 1..22: mol_type = other RNA
taaggacagctgttatttccta

SEQ ID NO: 853 - RNA, 22 nt, synthetic construct
  source 1..22: mol_type = other RNA
tgttatttcctaagtgatgatg

SEQ ID NO: 854 - RNA, 22 nt, synthetic construct
  source 1..22: mol_type = other RNA
cctaagtgatgatgtccaaaca

SEQ ID NO: 855 - RNA, 22 nt, synthetic construct
  source 1..22: mol_type = other RNA
gtgatgatgtccaaacatggca

SEQ ID NO: 856 - RNA, 22 nt, synthetic construct
  source 1..22: mol_type = other RNA
atgatgtccaaacatggcagga

SEQ ID NO: 857 - RNA, 22 nt, synthetic construct
  source 1..22: mol_type = other RNA
tgtttggacatcatcacttagg

SEQ ID NO: 858 - RNA, 22 nt, synthetic construct
  source 1..22: mol_type = other RNA
cctgccatgtttggacatcatc

SEQ ID NO: 859 - RNA, 22 nt, synthetic construct
  source 1..22: mol_type = other RNA
ttactctcctgccatgtttgga

SEQ ID NO: 860 - RNA, 22 nt, synthetic construct
  source 1..22: mol_type = other RNA
attttactctcctgccatgttt

SEQ ID NO: 861 - RNA, 22 nt, synthetic construct
  source 1..22: mol_type = other RNA
ttatttcctaagtgatgatgtc

SEQ ID NO: 862 - RNA, 22 nt, synthetic construct
  source 1..22: mol_type = other RNA
tcctaagtgatgatgtccaaac

SEQ ID NO: 863 - RNA, 22 nt, synthetic construct
  source 1..22: mol_type = other RNA
ccaaacatggcaggagagtaaa

SEQ ID NO: 864 - RNA, 22 nt, synthetic construct
  source 1..22: mol_type = other RNA
caaacatggcaggagagtaaaa

SEQ ID NO: 865 - RNA, 22 nt, synthetic construct
  source 1..22: mol_type = other RNA
atggcaggagagtaaaatggcc

SEQ ID NO: 866 - RNA, 22 nt, synthetic construct
  source 1..22: mol_type = other RNA
gcaggagagtaaaatggcctgt

SEQ ID NO: 867 - RNA, 22 nt, synthetic construct
  source 1..22: mol_type = other RNA
ggacatcatcacttaggaaata

SEQ ID NO: 868 - RNA, 22 nt, synthetic construct
  source 1..22: mol_type = other RNA
ttggacatcatcacttaggaaa

SEQ ID NO: 869 - RNA, 22 nt, synthetic construct
  source 1..22: mol_type = other RNA
actctcctgccatgtttggaca

SEQ ID NO: 870 - RNA, 22 nt, synthetic construct
  source 1..22: mol_type = other RNA
tttactctcctgccatgtttgg

SEQ ID NO: 871 - RNA, 22 nt, synthetic construct
  source 1..22: mol_type = other RNA
cattttactctcctgccatgtt

SEQ ID NO: 872 - RNA, 22 nt, synthetic construct
  source 1..22: mol_type = other RNA
gcataaggacagctgttatttc

SEQ ID NO: 873 - RNA, 22 nt, synthetic construct
  source 1..22: mol_type = other RNA
tttcctaagtgatgatgtccaa

SEQ ID NO: 874 - RNA, 22 nt, synthetic construct
  source 1..22: mol_type = other RNA
taagtgatgatgtccaaacatg

SEQ ID NO: 875 - RNA, 22 nt, synthetic construct
  source 1..22: mol_type = other RNA
tgatgatgtccaaacatggcag

SEQ ID NO: 876 - RNA, 22 nt, synthetic construct
  source 1..22: mol_type = other RNA
acttaggaaataacagctgtcc

SEQ ID NO: 877 - RNA, 22 nt, synthetic construct
  source 1..22: mol_type = other RNA
tgccatgtttggacatcatcac

SEQ ID NO: 878 - RNA, 22 nt, synthetic construct
  source 1..22: mol_type = other RNA
ctgccatgtttggacatcatca

SEQ ID NO: 879 - RNA, 22 nt, synthetic construct
  source 1..22: mol_type = other RNA
gccattttactctcctgccatg

SEQ ID NO: 880 - RNA, 23 nt, synthetic construct
  source 1..23: mol_type = other RNA
taagtgatgatgtccaaacatgg

SEQ ID NO: 881 - RNA, 23 nt, synthetic construct
  source 1..23: mol_type = other RNA
tgatgatgtccaaacatggcagg

SEQ ID NO: 882 - RNA, 23 nt, synthetic construct
  source 1..23: mol_type = other RNA
acaaatgtaagccttgtccaagg

SEQ ID NO: 883 - RNA, 23 nt, synthetic construct
  source 1..23: mol_type = other RNA
cttgtccaaggagatggatttgg

SEQ ID NO: 884 - RNA, 23 nt, synthetic construct
  source 1..23: mol_type = other RNA
gtaagccttgtccaaggagatgg

SEQ ID NO: 885 - RNA, 23 nt, synthetic construct
  source 1..23: mol_type = other RNA
aaggagatggatttggcataagg

SEQ ID NO: 886 - RNA, 23 nt, synthetic construct
  source 1..23: mol_type = other RNA
cccatgatggtagaaacacctgg

SEQ ID NO: 887 - RNA, 23 nt, synthetic construct
  source 1..23: mol_type = other RNA
ccatgatggtagaaacacctggg

SEQ ID NO: 888 - RNA, 23 nt, synthetic construct
  source 1..23: mol_type = other RNA
cacccctctctatcccatgatgg

SEQ ID NO: 889 - RNA, 23 nt, synthetic construct
  source 1..23: mol_type = other RNA
gttgtttatcttcaacaggctgg

SEQ ID NO: 890 - RNA, 23 nt, synthetic construct
  source 1..23: mol_type = other RNA
atgtttggacatcatcacttagg

SEQ ID NO: 891 - RNA, 23 nt, synthetic construct
  source 1..23: mol_type = other RNA
gctggcattctgagcagcacagg

SEQ ID NO: 892 - RNA, 23 nt, synthetic construct
  source 1..23: mol_type = other RNA
ttttgttgtttatcttcaacagg

SEQ ID NO: 893 - RNA, 23 nt, synthetic construct
  source 1..23: mol_type = other RNA
ttttactctcctgccatgtttgg

SEQ ID NO: 894 - protein, 378 aa, synthetic construct
MVLLWLTLLLIALPCLLQTKEDPNPPITNLRMKAKAQQLTWDLNRNVTDIECVKDADYSMPAVNNSYCQFGAISLCEVTNYTVQIANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 895 - protein, 378 aa, synthetic construct
MVLLWLTLLLIALPCLLQTKEDPNPPITNLRMKAKAQQLTWDLNRNVTDIECVKDADYSMPAVNNSYCQFGAISLCEVTNYTVQMANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 896 - protein, 378 aa, synthetic construct
MVLLWLTLLLIALPCLLQTKEDPNPPITNLRMKAKAQQLTWDLNRNVTDIECVKDADYSMPAVNNSYCQFGAISLCEVTNYTVHIANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 897 - protein, 378 aa, synthetic construct
MVLLWLTLLLIALPCLLQTKEDPNPPITNLRMKAKAQQLTWDLNRNVTDIECVKDADYSMPAVNNSYCQFGAISLCEVTNYTVHMANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT